SEQUENCE LISTING (41 sequences)

SEQ ID NO: 1 - DNA, 1842 bp, cytomegalovirus
tacgcgcaggcagcatggagtcgcgcggtcgccgttgtcccgaaatgatatccgtactgggtcccatttcggggcacgtgctgaaagccgtgtttagtcgcggcgacacgccggtgctgccgcacgagacgcgactcctgcagacgggtatccacgtgcgcgtgagccagccctcgctgatcctggtgtcgcagtacacgcccgactcgacgccatgccaccgcggcgacaatcagctgcaggtgcagcacacgtactttacgggcagcgaggtggagaacgtgtcggtcaacgtgcacaaccccacgggccggagcatctgccccagccaagagcccatgtcgatctatgtgtacgcgctgccgctcaagatgctgaacatccccagcatcaacgtgcaccactacccgtcggcggccgagcgcaaacaccgacacctgcccgtagctgacgctgtgattcacgcgtcgggcaagcagatgtggcaggcgcgtctcacggtctcgggactggcctggacgcgtcagcagaaccagtggaaagagcccgacgtctactacacgtcagcgttcgtgtttcccaccaaggacgtggcactgcggcacgtggtgtgcgcgcacgagctggtttgctccatggagaacacgcgcgcaaccaagatgcaggtgataggtgaccagtacgtcaaggtgtacctggagtccttctgcgaggacgtgccctccggcaagctctttatgcacgtcacgctgggctctgacgtggaagaggacctgacgatgacccgcaacccgcaacccttcatgcgcccccacgagcgcaacggctttacggtgttgtgtcccaaaaatatgataatcaaaccgggcaagatctcgcacatcatgctggatgtggcttttacctcacacgagcattttgggctgctgtgtcccaagagcatcccgggcctgagcatctcaggtaacctattgatgaacgggcagcagatcttcctggaggtgcaagcgatacgcgagaccgtggaactgcgtcagtacgatcccgtggctgcgctcttctttttcgatatcgacttgctgctgcagcgcgggcctcagtacagcgaacaccccaccttcaccagccagtatcgcatccagggcaagcttgagtaccgacacacctgggaccggcacgacgagggtgccgcccagggcgacgacgacgtctggaccagcggatcggactccgacgaggaactcgtaaccaccgagcgcaagacgccccgcgttaccggcggcggcgccatggcgggcgcctccacttccgcgggccgcaaccgcaaatcagcatcctcggcgacggcgtgcacggcgggcgttatgacacgcggccgccttaaggccgagtccaccgtcgcgcccgaagaggacaccgacgaggattccgacaacgaaatccacaatccggccgtgttcacctggccgccctggcaggccggcatcctggcccgcaacctggtgcccatggtggctacggttcagggtcagaatctgaagtaccaggagttcttctgggacgccaacgacatctaccgcatcttcgccgaattggaaggcgtatggcagcccgctgcgcaacccaaacgtcgccgccaccggcaagacgccttgcccgggccatgcatcgcctcgacgcccaaaaagcaccgaggttgagccacccgccgcgcacgcgcttaggacgactctataaaaacccacgtccactcagacacgcgacttttggccgccacacctgtcgccgctgctatatttgcgacagttgccggaacccttcccgacctcccacgaagacccg

SEQ ID NO: 2 - DNA, 1542 bp, cytomegalovirus
gcagtcaccgtccttgacacgatggagtcctctgccaagagaaagatggaccctgataatcctgacgagggcccttcctccaaggtgccacggcccgagacacccgtgaccaaggccacgacgttcctgcagactatgttgaggaaggaggttaacagtcagctgagtctgggagacccgctgtttccagagttggccgaagaatccctcaaaacttttgaacaagtgaccgaggattgcaacgagaaccccgagaaagatgtcctggcagaactcgtcaaacagattaaggttcgagtggacatggtgcggcatagaatcaaggagcacatgctgaaaaaatatacccagacggaagagaaattcactggcgcctttaatatgatgggaggatgtttgcagaatgccttagatatcttagataaggttcatgagcctttcgaggagatgaagtgtattgggctaactatgcagagcatgtatgagaactacattgtacctgaggataagcgggagatgtggatggcttgtattaaggagctgcatgatgtgagcaagggcgccgctaacaagttggggggtgcactgcaggctaaggcccgtgctaaaaaggatgaacttaggagaaagatgatgtatatgtgctacaggaatatagagttctttaccaagaactcagccttccctaagaccaccaatggctgcagtcaggccatggcggcactgcagaacttgcctcagtgctcccctgatgagattatggcttatgcccagaaaatatttaagattttggatgaggagagagacaaggtgctcacgcacattgatcacatatttatggatatcctcactacatgtgtggaaacaatgtgtaatgagtacaaggtcactagtgacgcttgtatgatgaccatgtacgggggcatctctctcttaagtgagttctgtcgggtgctgtgctgctatgtcttagaggagactagtgtgatgctggccaagcggcctctgataaccaagcctgaggttatcagtgtaatgaagcgccgcattgaggagatctgcatgaaggtctttgcccagtacattctgggggccgatcctctgagagtctgctctcctagtgtggatgacctacgggccatcgccgaggagtcagatgaggaagaggctattgtagcctacactttggccaccgctggtgtcagctcctctgattctctggtgtcacccccagagtcccctgtacccgcgactatccctctgtcctcagtaattgtggctgagaacagtgatcaggaagaaagtgagcagagtgatgaggaagaggaggagggtgctcaggaggagcgggaggacactgtgtctgtcaagtctgagccagtgtctgagatagaggaagttgccccagaggaagaggaggatggtgctgaggaacccaccgcctctggaggcaagagcacccaccctatggtgactagaagcaaggctgaccagtaaactattgtatatatatatcagttactgttatggatcccacgtcac

SEQ ID NO: 3 - DNA, 2347 bp, Artificial Sequence
  Fusion of the native sequences IE1 and truncated pp65mII.
gacacgatggagtcctctgccaagagaaagatggaccctgataatcctgacgagggcccttcctccaaggtgccacggcccgagacacccgtgaccaaggccacgacgttcctgcagactatgttgaggaaggaggttaacagtcagctgagtctgggagacccgctgtttccagagttggccgaagaatccctcaaaacttttgaacaagtgaccgaggattgcaacgagaaccccgagaaagatgtcctggcagaactcgtcaaacagattaaggttcgagtggacatggtgcggcatagaatcaaggagcacatgctgaaaaaatatacccagacggaagagaaattcactggcgcctttaatatgatgggaggatgtttgcagaatgccttagatatcttagataaggttcatgagcctttcgaggagatgaagtgtattgggctaactatgcagagcatgtatgagaactacattgtacctgaggataagcgggagatgtggatggcttgtattaaggagctgcatgatgtgagcaagggcgccgctaacaagttggggggtgcactgcaggctaaggcccgtgctaaaaaggatgaacttaggagaaagatgatgtatatgtgctacaggaatatagagttctttaccaagaactcagccttccctaagaccaccaatggctgcagtcaggccatggcggcactgcagaacttgcctcagtgctcccctgatgagattatggcttatgcccagaaaatatttaagattttggatgaggagagagacaaggtgctcacgcacattgatcacatatttatggatatcctcactacatgtgtggaaacaatgtgtaatgagtacaaggtcactagtgacgcttgtatgatgaccatgtacgggggcatctctctcttaagtgagttctgtcgggtgctgtgctgctatgtcttagaggagactagtgtgatgctggccaagcggcctctgataaccaagcctgaggttatcagtgtaatgaagcgccgcattgaggagatctgcatgaaggtctttgcccagtacattctgggggccgatcctctgagagtctgctctcctagtgtggatgacctacgggccatcgccgaggagtcagatgaggaagaggctattgtagcctacactttggccaccgctggtgtcagctcctctgattctctggtgtcacccccagagtcccctgtacccgcgactatccctctgtcctcagtaattgtggctgagaacagtgatcaggaagaaagtgagcagagtgatgaggaagaggaggagggtgctcaggaggagcgggaggacactgtgtctgtcaagtctgagccagtgtctgagatagaggaagttgccccagaggaagaggaggatggtgctgaggaacccaccgcctctggaggcaagagcacccaccctatggtgactagaagcaaggctgcttttacctcacacgagcattttgggctgctgtgtcccaagagcatcccgggcctgagcatctcaggtaacctattgatgaacgggcagcagatcttcctggaggtgcaagcgatacgcgagaccgtggaactgcgtcagtacgatcccgtggctgcgctcttctttttcgatatcgacttgctgctgcagcgcgggcctcagtacagcgaacaccccaccttcaccagccagtatcgcatccagggcaagcttgagtaccgacacacctgggaccggcacgacgagggtgccgcccagggcgacgacgacgtctggaccagcggatcggactccgacgaggaactcgtaaccaccgagcgcaagacgccccgcgttaccggcggcggcgccatggcgggcgcctccacttccgcgggccgcaaacgcaaatcagcatcctcggcgacggcgtgcacggcgggcgttatgacacgcggccgccttaaggccgagtccaccgtcgcgcccgaagaggacaccgacgaggattccgacaacgaaatccacaatccggccgtgttcacctggccgccctggcaggccggcatcctggcccgcaacctggtgcccatggtggctacggttcagggtcagaatctgaagtaccaggagttcttctgggacgccaacgacatctaccgcatcttcgccgaattggaaggcgtatggcagcccgctgcgcaacccaaacgtcgccgccaccggcaagacgccttgcccgggccatgcatcgcctcgacgcccaaaaagcaccgaggttgagccacccgccgcgcacgcgcttaggacgactctataaaaacccacgtccactcagacacgcgacttttgg

SEQ ID NO: 4 - DNA, 2347 bp, Artificial Sequence; identical to SEQ ID NO: 3
  Top strand of the fusion of the native sequences IE1 andtruncated pp65mII.

SEQ ID NO: 5 - protein, 756 aa, Artificial Sequence
  Protein sequence corresponding to CMV-IEpp65MII, a fusion of the native sequences IE1 and truncated pp65mII.
MESSAKRKMDPDNPDEGPSSKVPRPETPVTKATTFLQTMLRKEVNSQLSLGDPLFPELAEESLKTFEQVTEDCNENPEKDVLAELVKQIKVRVDMVRHRIKEHMLKKYTQTEEKFTGAFNMMGGCLQNALDILDKVHEPFEEMKCIGLTMQSMYENYIVPEDKREMWMACIKELHDVSKGAANKLGGALQAKARAKKDELRRKMMYMCYRNIEFFTKNSAFPKTTNGCSQAMAALQNLPQCSPDEIMAYAQKIFKILDEERDKVLTHIDHIFMDILTTCVETMCNEYKVTSDACMMTMYGGISLLSEFCRVLCCYVLEETSVMLAKRPLITKPEVISVMKRRIEEICMKVFAQYILGADPLRVCSPSVDDLRAIAEESDEEEAIVAYTLATAGVSSSDSLVSPPESPVPATIPLSSVIVAENSDQEESEQSDEEEEEGAQEEREDTVSVKSEPVSEIEEVAPEEEEDGAEEPTASGGKSTHPMVTRSKAAFTSHEHFGLLCPKSIPGLSISGNLLMNGQQIFLEVQAIRETVELRQYDPVAALFFFDIDLLLQRGPQYSEHPTFTSQYRIQGKLEYRHTWDRHDEGAAQGDDDVWTSGSDSDEELVTTERKTPRVTGGGAMAGASTSAGRKRKSASSATACTAGVMTRGRLKAESTVAPEEDTDEDSDNEIHNPAVFTWPPWQAGILARNLVPMVATVQGQNLKYQEFFWDANDIYRIFAELEGVWQPAAQPKRRRHRQDALPGPCIASTPKKHRG

SEQ ID NO: 6 - DNA, 9167 bp, Artificial Sequence
  Combined fusion of IE1 and pp65mII plus a truncated gB which contains the immunologic domains.
gactcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagtgtaagtaccgcctatagagtctataggcccacccccttggcttcttatgcatgctatactgtttttggcttggggtctatacacccccgcttcctcatgttataggtgatggtatagcttagcctataggtgtgggttattgaccattattgaccactcccctattggtgacgatactttccattactaatccataacatggctctttgccacaactctctttattggctatatgccaatacactgtccttcagagactgacacggactctgtatttttacaggatggggtctcatttattatttacaaattcacatatacaacaccaccgtccccagtgcccgcagtttttattaaacataacgtgggatctccacgcgaatctcgggtacgtgttccggacatgggctcttctccggtagcggcggagcttctacatccgagccctgctcccatgcctccagcgactcatggtcgctcggcagctccttgctcctaacagtggaggccagacttaggcacagcacgatgcccaccaccaccagtgtgccgcacaaggccgtggcggtagggtatgtgtctgaaaatgagctcggggagcgggcttgcaccgctgacgcatttggaagacttaaggcagcggcagaagaagatgcaggcagctgagttgttgtgttctgataagagtcagaggtaactcccgttgcggtgctgttaacggtggagggcagtgtagtctgagcagtactcgttgctgccgcgcgcgccaccagacataatagctgacagactaacagactgttcctttccatgggtcttttctgcagcgtttaaacttaagcttggtaccgagctcggatccactagtccagtgtggtggaattcgacacgatggagtcctctgccaagagaaagatggaccctgataatcctgacgagggcccttcctccaaggtgccacgtaggcccgagacacccgtgagcccgagacacccgtgaccaaggccacgacgttcctgcagactatgttgaggaaggaggttaacagtcagctgagtctgggagacccgctgtttccagagttggccgaagaatccctcaaaacttttgaacaagtgaccgaggattgcaacgagaaccccgagaaagatgtcctggcagaactcgtcaaacagattaaggttcgagtggacatggtgcggcatagaatcaaggagcacatgctgaaaaaatatacccagacggaagagaaattcactggcgcctttaatatgatgggaggatgtttgcagaatgccttagatatcttagataaggttcatgagcctttcgaggagatgaagtgtattgggctaactatgcagagcatgtatgagaactacattgtacctgaggataagcgggagatgtggatggcttgtattaaggagctgcatgatgtgagcaagggcgccgctaacaagttggggggtgcactgcaggctaaggcccgtgctaaaaaggatgaacttaggagaaagatgatgtatatgtgctacaggaatatagagttctttaccaagaactcagccttccctaagaccaccaatggctgcagtcaggccatggcggcactgcagaacttgcctcagtgctcccctgatgagattatggcttatgcccagaaaatatttaagattttggatgaggagagagacaaggtgctcacgcacattgatcacatatttatggatatcctcactacatgtgtggaaacaatgtgtaatgagtacaaggtcactagtgacgcttgtatgatgaccatgtacgggggcatctctctcttaagtgagttctgtcgggtgctgtgctgctatgtcttagaggagactagtgtgatgctggccaagcggcctctgataaccaagcctgaggttatcagtgtaatgaagcgccgcattgaggagatctgcatgaaggtctttgcccagtacattctgggggccgatcctctgagagtctgctctcctagtgtggatgacctacgggccatcgccgaggagtcagatgaggaagaggctattgtagcctacactttggccaccgctggtgtcagctcctctgattctctggtgtcacccccagagtcccctgtacccgcgactatccctctgtcctcagtaattgtggctgagaacagtgatcaggaagaaagtgagcagagtgatgaggaagaggaggagggtgctcaggaggagcgggaggacactgtgtctgtcaagtctgagccagtgtctgagatagaggaagttgccccagaggaagaggaggatggtgctgaggaacccaccgcctctggaggcaagagcacccaccctatggtgactagaagcaaggctgcttttacctcacacgagcattttgggctgctgtgtcccaagagcatcccgggcctgagcatctcaggtaacctattgatgaacgggcagcagatcttcctggaggtgcaagcgatacgcgagaccgtggaactgcgtcagtacgatcccgtggctgcgctcttctttttcgatatcgacttgctgctgcagcgcgggcctcagtacagcgaacaccccaccttcaccagccagtatcgcatccagggcaagcttgagtaccgacacacctgggaccggcacgacgagggtgccgcccagggcgacgacgacgtctggaccagcggatcggactccgacgaggaactcgtaaccaccgagcgcaagacgccccgcgttaccggcggcggcgccatggcgggcgcctccacttccgcgggccgcaaacgcaaatcagcatcctcggcgacggcgtgcacggcgggcgttatgacacgcggccgccttaaggccgagtccaccgtcgcgcccgaagaggacaccgacgaggattccgacaacgaaatccacaatccggccgtgttcacctggccgccctggcaggccggcatcctggcccgcaacctggtgcccatggtggctacggttcagggtcagaatctgaagtaccaggagttcttctgggacgccaacgacatctaccgcatcttcgccgaattggaaggcgtatggcagcccgctgcgcaacccaaacgtcgccgccaccggcaagacgccttgcccgggccatgcatcgcctcgacgcccaaaaagcaccgaggttgagccacccgccgcgcacgcgcttaggacgactctataaaaacccacgtccactcagacacgcgacttttggtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctactgggcggttttatggacagcaagcgaaccggaattgccagctggggcgccctctggtaaggttgggaagccctgcaaagtaaactggatggctttctcgccgccaaggatctgatggcgcaggggatcaagctctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattattaacgcttacaatttcctgatgcggtattttctccttacgcatctgtgcggtatttcacaccgcatacaggtggcacttttcggggaaatgtgcgcggaacccctatttgtttatttttctaaatacattcaaatatgtatccgctcatgagacaataaccctgataaatgcttcaataatagcaccaagtgcaggtgccagaacatttctctatcgaaggatctgcgatcgctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctacgtaagtgatatctactagatttatcaaaaagagtgttgacttgtgagcgctcacaattgatacttagattcatcgagagggacacgtcgactactaaccttcttctctttcctacagctgagatcaccggcgaaggaggcctrgatcycggatccgaagcttaacatggaatccaggatctggtgcctggtagtctgcgttaacctgtgtatcgtctgtctgggtgctgcggtttcctcttctagtacttcccatgcaacttcttctactcacaatggaagccatacttctcgtacgacgtctgctcaaacccggtcagtctattctcaacacgtaacgtcttctgaagccgtcagtcatagagccaacgagactatctacaacactaccctcaagtacggagatgtggtgggagtcaacactaccaagtacccctatcgcgtgtgttctatggcccagggtacggatcttattcgctttgaacgtaatatcatctgcacctcgatgaagcctatcaatgaagacttggatgagggcatcatggtggtctacaagcgcaacatcgtggcgcacacctttaaggtacgggtctaccaaaaggttttgacgtttcgtcgtagctacgcttacatctacaccacttatctgctgggcagcaatacggaatacgtggcgcctcctatgtgggagattcatcacatcaacaagtttgctcaatgctacagttcctacagccgcgttataggaggcacggttttcgtggcatatcatagggacagttatgaaaacaaaaccatgcaattaattcccgacgattattccaacacccacagtacccgttacgtgacggtcaaggatcagtggcacagccgcggcagcacctggctctatcgtgagacctgtaatctgaactgtatgctgaccatcactactgcgcgctccaagtatccttatcatttttttgcaacttccacgggtgatgtggtttacatttctcctttctacaacggaaccaatcgcaatgccagctactttggagaaaacgccgacaagtttttcattttcccgaactacaccatcgtttccgactttggaagacccaacgctgcgccagaaacccataggttggtggcttttctcgaacgtgccgactcggtgatctcttgggatatacaggacgagaagaatgtcacctgccagctcaccttctgggaagcctcggaacgtactatccgttccgaagccgaagactcgtaccacttttcttctgccaaaatgactgcaacttttctgtctaagaaacaagaagtgaacatgtccgactccgcgctggactgcgtacgtgatgaggctataaataagttacagcagattttcaatacttcatacaatcaaacatatgaaaaatacggaaacgtgtccgtcttcgaaaccagcggcggtctggtggtgttctggcaaggcatcaagcaaaaatctttggtggaattggaacgtttggccaatcgatccagtctgaatatcactcataggaccagaagaagtacgagtgacaataatacaactcanttgtccagcatggaatcggtgcacaatctggtctacgcccagctgcagttcacctatgacacgttgcgcggttacatcaaccgggcgctggcgcaaatcgcagaagcctggtgtgtggatcaacggcgcaccctagaggtcttcaaggaactcagcaagatcaacccgtcagccattctctcggccatttacaacaaaccgattgccgcgcgtttcatgggtgatgtcttgggcctggccagctgcgtgaccatcaaccaaaccagcgtcaaggtgctgcgtgatatgaacgtgaaggaatcgccaggacgctgctactcacgacccgtggtcatctttaatttcgccaacagctcgtacgtgcagtacggtcaactgggcgaggacaacgaaatcctgttgggcaaccaccgcactgaggaatgtcagcttcccagcctcaagatcttcatcgccgggaactcggcctacgagtacgtggactacctcttcaaacgcatgattgacctcagcagtatctccaccgtcgacagcatgatcgccctggatatcgacccgctggcgagctccyctagrtcgacatgataagatacattgatgagtttggacaaaccacaactagaatgcagtgaaaaaaatgctttatttgtgaaatttgtgatgctattgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtagatcatttaaatgttagcgaagaacatgtgagcgtgctaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctgggcttttgctggccttttgctcacatgttctt

SEQ ID NO: 7 - DNA, 1716 bp, Artificial Sequence
  Full length CMV pp65mII with NLS-KO
tacgcgcaggcagcatggagtcgcgcggtcgccgttgtcccgaaatgatatccgtactgggtcccatttcggggcacgtgctgaaagccgtgtttagtcgcggcgacacgccggtgctgccgcacgagacgcgactcctgcagacgggtatccacgtgcgcgtgagccagccctcgctgatcctggtgtcgcagtacacgcccgactcgacgccatgccaccgcggcgacaatcagctgcaggtgcagcacacgtactttacgggcagcgaggtggagaacgtgtcggtcaacgtgcacaaccccacgggccggagcatctgccccagccaagagcccatgtcgatctatgtgtacgcgctgccgctcaagatgctgaacatccccagcatcaacgtgcaccactacccgtcggcggccgagcgcaaacaccgacacctgcccgtagctgacgctgtgattcacgcgtcgggcaagcagatgtggcaggcgcgtctcacggtctcgggactggcctggacgcgtcagcagaaccagtggaaagagcccgacgtctactacacgtcagcgttcgtgtttcccaccaaggacgtggcactgcggcacgtggtgtgcgcgcacgagctggtttgctccatggagaacacgcgcgcaaccaagatgcaggtgataggtgaccagtacgtcaaggtgtacctggagtccttctgcgaggacgtgccctccggcaagctctttatgcacgtcacgctgggctctgacgtggaagaggacctgacgatgacccgcaacccgcaacccttcatgcgcccccacgagcgcaacggctttacggtgttgtgtcccaaaaatatgataatcaaaccgggcaagatctcgcacatcatgctggatgtggcttttacctcacacgagcattttgggctgctgtgtcccaagagcatcccgggcctgagcatctcaggtaacctattgatgaacgggcagcagatcttcctggaggtgcaagcgatacgcgagaccgtggaactgcgtcagtacgatcccgtggctgcgctcttctttttcgatatcgacttgctgctgcagcgcgggcctcagtacagcgaacaccccaccttcaccagccagtatcgcatccagggcaagcttgagtaccgacacacctgggaccggcacgacgagggtgccgcccagggcgacgacgacgtctggaccagcggatcggactccgacgaggaactcgtaaccaccgagcgcaagacgccccgcgttaccggcggcggcgccatggcgggcgcctccacttccgcgggccgcaaccgcaaatcagcatcctcggcgacggcgtgcacggcgggcgttatgacacgcggccgccttaaggccgagtccaccgtcgcgcccgaagaggacaccgacgaggattccgacaacgaaatccacaatccggccgtgttcacctggccgccctggcaggccggcatcctggcccgcaacctggtgcccatggtggctacggttcagggtcagaatctgaagtaccaggagttcttctgggacgccaacgacatctaccgcatcttcgccgaattggaaggcgtatggcagctgacccacgtccactcagacacgcgacttttggccgccacacctgtcgccgctgctatatttgcgacagttgccggaacccttcccgacctcccacgaagacccg

SEQ ID NO: 8 - DNA, 1716 bp, Artificial Sequence; identical to SEQ ID NO: 7
  Full length CMV pp65mII with NLS-KO

SEQ ID NO: 9 - protein, 532 aa, Artificial Sequence
  Protein sequence generated by full length CMV pp65mII with NLS-KO
MESRGRRCPEMISVLGPISGHVLKAVFSRGDTPVLPHETRLLQTGIHVRVSQPSLILVSQYTPDSTPCHRGDNQLQVQHTYFTGSEVENVSVNVHNPTGRSICPSQEPMSIYVYALPLKMLNIPSINVHHYPSAAERKHRHLPVADAVIHASGKQMWQARLTVSGLAWTRQQNQWKEPDVYYTSAFVFPTKDVALRHVVCAHELVCSMENTRATKMQVIGDQYVKVYLESFCEDVPSGKLFMHVTLGSDVEEDLTMTRNPQPFMRPHERNGFTVLCPKNMIIKPGKISHIMLDVAFTSHEHFGLLCPKSIPGLSISGNLLMNGQQIFLEVQAIRETVELRQYDPVAALFFFDIDLLLQRGPQYSEHPTFTSQYRIQGKLEYRHTWDRHDEGAAQGDDDVWTSGSDSDEELVTTERKTPRVTGGGAMAGASTSAGRNRKSASSATACTAGVMTRGRLKAESTVAPEEDTDEDSDNEIHNPAVFTWPPWQAGILARNLVPMVATVQGQNLKYQEFFWDANDIYRIFAELEGVWQ

SEQ ID NO: 10 - DNA, 1503 bp, Artificial Sequence
  Full-length CMV IE1 with NLS-KO
gcagtcaccgtccttgacacgatggagtcctctgccaagagaaagatggaccctgataatcctgacgagggcccttcctccaaggtgccacggcccgagacacccgtgaccaaggccacgacgttcctgcagactatgttgaggaaggaggttaacagtcagctgagtctgggagacccgctgtttccagagttggccgaagaatccctcaaaacttttgaacaagtgaccgaggattgcaacgagaaccccgagaaagatgtcctggcagaactcgtcaaacagattaaggttcgagtggacatggtgcggcatagaatcaaggagcacatgctgaaaaaatatacccagacggaagagaaattcactggcgcctttaatatgatgggaggatgtttgcagaatgccttagatatcttagataaggttcatgagcctttcgaggagatgaagtgtattgggctaactatgcagagcatgtatgagaactacattgtacctgaggataagcgggagatgtggatggcttgtattaaggagctgcatgatgtgagcaagggcgccgctaacaagttggggggtgcactgcaggctaaggcccgtgctaaaaaggatgaacttaggagaaagatgatgtatatgtgctacaggaatatagagttctttaccaagaactcagccttccctaagaccaccaatggctgcagtcaggccatggcggcactgcagaacttgcctcagtgctcccctgatgagattatggcttatgcccagaaaatatttaagattttggatgaggagagagacaaggtgctcacgcacattgatcacatatttatggatatcctcactacatgtgtggaaacaatgtgtaatgagtacaaggtcactagtgacgcttgtatgatgaccatgtacgggggcatctctctcttaagtgagttctgtcgggtgctgtgctgctatgtcttagaggagactagtgtgatgctggccgtcgacgtcgacattgaggagatctgcatgaaggtctttgcccagtacattctgggggccgatcctctgagagtctgctctcctagtgtggatgacctacgggccatcgccgaggagtcagatgaggaagaggctattgtagcctacactttggccaccgctggtgtcagctcctctgattctctggtgtcacccccagagtcccctgtacccgcgactatccctctgtcctcagtaattgtggctgagaacagtgatcaggaagaaagtgagcagagtgatgaggaagaggaggagggtgctcaggaggagcgggaggacactgtgtctgtcaagtctgagccagtgtctgagatagaggaagttgccccagaggaagaggaggatggtgctgaggaacccaccgcctctggaggcaagagcacccaccctatggtgactagaagcaaggctgaccagtaaactattgtatatatatatcagttactgttatggatcccacgtcac

SEQ ID NO: 11 - DNA, 1503 bp, Artificial Sequence; identical to SEQ ID NO: 10
  Full-length CMV IE1 with NLS-KO, single stranded.

SEQ ID NO: 12 - protein, 478 aa, Artificial Sequence
  Protein sequence generated by full-length CMV IE1 with NLS-KO
MESSAKRKMDPDNPDEGPSSKVPRPETPVTKATTFLQTMLRKEVNSQLSLGDPLFPELAEESLKTFEQVTEDCNENPEKDVLAELVKQIKVRVDMVRHRIKEHMLKKYTQTEEKFTGAFNMMGGCLQNALDILDKVHEPFEEMKCIGLTMQSMYENYIVPEDKREMWMACIKELHDVSKGAANKLGGALQAKARAKKDELRRKMMYMCYRNIEFFTKNSAFPKTTNGCSQAMAALQNLPQCSPDEIMAYAQKIFKILDEERDKVLTHIDHIFMDILTTCVETMCNEYKVTSDACMMTMYGGISLLSEFCRVLCCYVLEETSVMLAVDVDIEEICMKVFAQYILGADPLRVCSPSVDDLRAIAEESDEEEAIVAYTLATAGVSSSDSLVSPPESPVPATIPLSSVIVAENSDQEESEQSDEEEEEGAQEEREDTVSVKSEPVSEIEEVAPEEEEDGAEEPTASGGKSTHPMVTRSKADQ

SEQ ID NO: 13 - DNA, 2160 bp, Artificial Sequence
  Fusion of IE1 and the truncated pp65mII with NLS-KO in each.
gacacgatggagtcctctgccaagagaaagatggaccctgataatcctgacgagggcccttcctccaaggtgccacggcccgagacacccgtgaccaaggccacgacgttcctgcagactatgttgaggaaggaggttaacagtcagctgagtctgggagacccgctgtttccagagttggccgaagaatccctcaaaacttttgaacaagtgaccgaggattgcaacgagaaccccgagaaagatgtcctggcagaactcgtcaaacagattaaggttcgagtggacatggtgcggcatagaatcaaggagcacatgctgaaaaaatatacccagacggaagagaaattcactggcgcctttaatatgatgggaggatgtttgcagaatgccttagatatcttagataaggttcatgagcctttcgaggagatgaagtgtattgggctaactatgcagagcatgtatgagaactacattgtacctgaggataagcgggagatgtggatggcttgtattaaggagctgcatgatgtgagcaagggcgccgctaacaagttggggggtgcactgcaggctaaggcccgtgctaaaaaggatgaacttaggagaaagatgatgtatatgtgctacaggaatatagagttctttaccaagaactcagccttccctaagaccaccaatggctgcagtcaggccatggcggcactgcagaacttgcctcagtgctcccctgatgagattatggcttatgcccagaaaatatttaagattttggatgaggagagagacaaggtgctcacgcacattgatcacatatttatggatatcctcactacatgtgtggaaacaatgtgtaatgagtacaaggtcactagtgacgcttgtatgatgaccatgtacgggggcatctctctcttaagtgagttctgtcgggtgctgtgctgctatgtcttagaggagactagtgtgatgctggccgtcgacgtcattgaggagatctgcatgaaggtctttgcccagtacattctgggggccgatcctctgagagtctgctctcctagtgtggatgacctacgggccatcgccgaggagtcagatgaggaagaggctattgtagcctacactttggccaccgctggtgtcagctcctctgattctctggtgtcacccccagagtcccctgtacccgcgactatccctctgtcctcagtaattgtggctgagaacagtgatcaggaagaaagtgagcagagtgatgaggaagaggaggagggtgctcaggaggagcgggaggacactgtgtctgtcaagtctgagccagtgtctgagatagaggaagttgccccagaggaagaggaggatggtgctgaggaacccaccgcctctggaggcaagagcacccaccctatggtgactagaagcaaggctgcttttacctcacacgagcattttgggctgctgtgtcccaagagcatcccgggcctgagcatctcaggtaacctattgatgaacgggcagcagatcttcctggaggtgcaagcgatacgcgagaccgtggaactgcgtcagtacgatcccgtggctgcgctcttctttttcgatatcgacttgctgctgcagcgcgggcctcagtacagcgaacaccccaccttcaccagccagtatcgcatccagggcaagcttgagtaccgacacacctgggaccggcacgacgagggtgccgcccagggcgacgacgacgtctggaccagcggatcggactccgacgaggaactcgtaaccaccgagcgcaagacgccccgcgttaccggcggcggcgccatggcgggcgcctccacttccgcgggccgcaaacgcaaatcagcatcctcggcgacggcgtgcacggcgggcgttatgacacgcggccgccttaaggccgagtccaccgtcgcgcccgaagaggacaccgacgaggattccgacaacgaaatccacaatccggccgtgttcacctggccgccctggcaggccggcatcctggcccgcaacctggtgcccatggtggctacggttcagggtcagaatctgaagtaccaggagttcttctgggacgccaacgacatctaccgcatcttcgccgaattggaaggcgtatggcagcccgctgcgcaatga

SEQ ID NO: 14 - DNA, 2160 bp, Artificial Sequence; identical to SEQ ID NO: 13
  Fusion of IE1 and the truncated pp65mII with NLS-KO in each, single stranded.

SEQ ID NO: 15 - protein, 717 aa, Artificial Sequence
  Protein sequence generated by fusion of IE1 and the truncatedpp65mII with NLS-KO in each.
MESSAKRKMDPDNPDEGPSSKVPRPETPVTKATTFLQTMLRKEVNSQLSLGDPLFPELAEESLKTFEQVTEDCNENPEKDVLAELVKQIKVRVDMVRHRIKEHMLKKYTQTEEKFTGAFNMMGGCLQNALDILDKVHEPFEEMKCIGLTMQSMYENYIVPEDKREMWMACIKELHDVSKGAANKLGGALQAKARAKKDELRRKMMYMCYRNIEFFTKNSAFPKTTNGCSQAMAALQNLPQCSPDEIMAYAQKIFKILDEERDKVLTHIDHIFMDILTTCVETMCNEYKVTSDACMMTMYGGISLLSEFCRVLCCYVLEETSVMLAVDVIEEICMKVFAQYILGADPLRVCSPSVDDLRAIAEESDEEEAIVAYTLATAGVSSSDSLVSPPESPVPATIPLSSVIVAENSDQEESEQSDEEEEEGAQEEREDTVSVKSEPVSEIEEVAPEEEEDGAEEPTASGGKSTHPMVTRSKAAFTSHEHFGLLCPKSIPGLSISGNLLMNGQQIFLEVQAIRETVELRQYDPVAALFFFDIDLLLQRGPQYSEHPTFTSQYRIQGKLEYRHTWDRHDEGAAQGDDDVWTSGSDSDEELVTTERKTPRVTGGGAMAGASTSAGRKRKSASSATACTAGVMTRGRLKAESTVAPEEDTDEDSDNEIHNPAVFTWPPWQAGILARNLVPMVATVQGQNLKYQEFFWDANDIYRIFAELEGVWQPAAQ

SEQ ID NO: 16 - DNA, 9033 bp, Artificial Sequence
  Fusion of IE1 and the truncated pp65mII with NLS-KO in each, plus a truncated gB which contains the immunologic domains.
gactcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagtgtaagtaccgcctatagagtctataggcccacccccttggcttcttatgcatgctatactgtttttggcttggggtctatacacccccgcttcctcatgttataggtgatggtatagcttagcctataggtgtgggttattgaccattattgaccactcccctattggtgacgatactttccattactaatccataacatggctctttgccacaactctctttattggctatatgccaatacactgtccttcagagactgacacggactctgtatttttacaggatggggtctcatttattatttacaaattcacatatacaacaccaccgtccccagtgcccgcagtttttattaaacataacgtgggatctccacgcgaatctcgggtacgtgttccggacatgggctcttctccggtagcggcggagcttctacatccgagccctgctcccatgcctccagcgactcatggtcgctcggcagctccttgctcctaacagtggaggccagacttaggcacagcacgatgcccaccaccaccagtgtgccgcacaaggccgtggcggtagggtatgtgtctgaaaatgagctcggggagcgggcttgcaccgctgacgcatttggaagacttaaggcagcggcagaagaagatgcaggcagctgagttgttgtgttctgataagagtcagaggtaactcccgttgcggtgctgttaacggtggagggcagtgtagtctgagcagtactcgttgctgccgcgcgcgccaccagacataatagctgacagactaacagactgttcctttccatgggtcttttctgcagcgtttaaacttaagcttggtaccgagctcggatccactagtccagtgtggtggaattcgacacgatggagtcctctgccaagagaaagatggaccctgataatcctgacgagggcccttcctccaaggtgccacgtaggcccgagacacccgtgagcccgagacacccgtgaccaaggccacgacgttcctgcagactatgttgaggaaggaggttaacagtcagctgagtctgggagacccgctgtttccagagttggccgaagaatccctcaaaacttttgaacaagtgaccgaggattgcaacgagaaccccgagaaagatgtcctggcagaactcgtcaaacagattaaggttcgagtggacatggtgcggcatagaatcaaggagcacatgctgaaaaaatatacccagacggaagagaaattcactggcgcctttaatatgatgggaggatgtttgcagaatgccttagatatcttagataaggttcatgagcctttcgaggagatgaagtgtattgggctaactatgcagagcatgtatgagaactacattgtacctgaggataagcgggagatgtggatggcttgtattaaggagctgcatgatgtgagcaagggcgccgctaacaagttggggggtgcactgcaggctaaggcccgtgctaaaaaggatgaacttaggagaaagatgatgtatatgtgctacaggaatatagagttctttaccaagaactcagccttccctaagaccaccaatggctgcagtcaggccatggcggcactgcagaacttgcctcagtgctcccctgatgagattatggcttatgcccagaaaatatttaagattttggatgaggagagagacaaggtgctcacgcacattgatcacatatttatggatatcctcactacatgtgtggaaacaatgtgtaatgagtacaaggtcactagtgacgcttgtatgatgaccatgtacgggggcatctctctcttaagtgagttctgtcgggtgctgtgctgctatgtcttagaggagactagtgtgatgctggccattgaggagatctgcatgaaggtctttgcccagtacattctgggggccgatcctctgagagtctgctctcctagtgtggatgacctacgggccatcgccgaggagtcagatgaggaagaggctattgtagcctacactttggccaccgctggtgtcagctcctctgattctctggtgtcacccccagagtcccctgtacccgcgactatccctctgtcctcagtaattgtggctgagaacagtgatcaggaagaaagtgagcagagtgatgaggaagaggaggagggtgctcaggaggagcgggaggacactgtgtctgtcaagtctgagccagtgtctgagatagaggaagttgccccagaggaagaggaggatggtgctgaggaacccaccgcctctggaggcaagagcacccaccctatggtgactagaagcaaggctgcttttacctcacacgagcattttgggctgctgtgtcccaagagcatcccgggcctgagcatctcaggtaacctattgatgaacgggcagcagatcttcctggaggtgcaagcgatacgcgagaccgtggaactgcgtcagtacgatcccgtggctgcgctcttctttttcgatatcgacttgctgctgcagcgcgggcctcagtacagcgaacaccccaccttcaccagccagtatcgcatccagggcaagcttgagtaccgacacacctgggaccggcacgacgagggtgccgcccagggcgacgacgacgtctggaccagcggatcggactccgacgaggaactcgtaaccaccgagcgcaagacgccccgcgttaccggcggcggcgccatggcgggcgcctccacttccgcgggccgcaaacgcaaatcagcatcctcggcgacggcgtgcacggcgggcgttatgacacgcggccgccttaaggccgagtccaccgtcgcgcccgaagaggacaccgacgaggattccgacaacgaaatccacaatccggccgtgttcacctggccgccctggcaggccggcatcctggcccgcaacctggtgcccatggtggctacggttcagggtcagaatctgaagtaccaggagttcttctgggacgccaacgacatctaccgcatcttcgccgaattggaaggcgtatggcagcggttgagccacccgccgcgcacgcgcttaggacgactctataaaaacccacgtccactcagacacgcgacttttggtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctactgggcggttttatggacagcaagcgaaccggaattgccagctggggcgccctctggtaaggttgggaagccctgcaaagtaaactggatggctttctcgccgccaaggatctgatggcgcaggggatcaagctctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattattaacgcttacaatttcctgatgcggtattttctccttacgcatctgtgcggtatttcacaccgcatacaggtggcacttttcggggaaatgtgcgcggaacccctatttgtttatttttctaaatacattcaaatatgtatccgctcatgagacaataaccctgataaatgcttcaataatagcaccaagtgcaggtgccagaacatttctctatcgaaggatctgcgatcgctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctacgtaagtgatatctactagatttatcaaaaagagtgttgacttgtgagcgctcacaattgatacttagattcatcgagagggacacgtcgactactaaccttcttctctttcctacagctgagatcaccggcgaaggaggcctrgatcycggatccgaagcttaacatggaatccaggatctggtgcctggtagtctgcgttaacctgtgtatcgtctgtctgggtgctgcggtttcctcttctagtacttcccatgcaacttcttctactcacaatggaagccatacttctcgtacgacgtctgctcaaacccggtcagtctattctcaacacgtaacgtcttctgaagccgtcagtcatagagccaacgagactatctacaacactaccctcaagtacggagatgtggtgggagtcaacactaccaagtacccctatcgcgtgtgttctatggcccagggtacggatcttattcgctttgaacgtaatatcatctgcacctcgatgaagcctatcaatgaagacttggatgagggcatcatggtggtctacaagcgcaacatcgtggcgcacacctttaaggtacgggtctaccaaaaggttttgacgtttcgtcgtagctacgcttacatctacaccacttatctgctgggcagcaatacggaatacgtggcgcctcctatgtgggagattcatcacatcaacaagtttgctcaatgctacagttcctacagccgcgttataggaggcacggttttcgtggcatatcatagggacagttatgaaaacaaaaccatgcaattaattcccgacgattattccaacacccacagtacccgttacgtgacggtcaaggatcagtggcacagccgcggcagcacctggctctatcgtgagacctgtaatctgaactgtatgctgaccatcactactgcgcgctccaagtatccttatcatttttttgcaacttccacgggtgatgtggtttacatttctcctttctacaacggaaccaatcgcaatgccagctactttggagaaaacgccgacaagtttttcattttcccgaactacaccatcgtttccgactttggaagacccaacgctgcgccagaaacccataggttggtggcttttctcgaacgtgccgactcggtgatctcttgggatatacaggacgagaagaatgtcacctgccagctcaccttctgggaagcctcggaacgtactatccgttccgaagccgaagactcgtaccacttttcttctgccaaaatgactgcaacttttctgtctaagaaacaagaagtgaacatgtccgactccgcgctggactgcgtacgtgatgaggctataaataagttacagcagattttcaatacttcatacaatcaaacatatgaaaaatacggaaacgtgtccgtcttcgaaaccagcggcggtctggtggtgttctggcaaggcatcaagcaaaaatctttggtggaattggaacgtttggccaatcgatccagtctgaatatcactcataggaccagaagaagtacgagtgacaataatacaactcanttgtccagcatggaatcggtgcacaatctggtctacgcccagctgcagttcacctatgacacgttgcgcggttacatcaaccgggcgctggcgcaaatcgcagaagcctggtgtgtggatcaacggcgcaccctagaggtcttcaaggaactcagcaagatcaacccgtcagccattctctcggccatttacaacaaaccgattgccgcgcgtttcatgggtgatgtcttgggcctggccagctgcgtgaccatcaaccaaaccagcgtcaaggtgctgcgtgatatgaacgtgaaggaatcgccaggacgctgctactcacgacccgtggtcatctttaatttcgccaacagctcgtacgtgcagtacggtcaactgggcgaggacaacgaaatcctgttgggcaaccaccgcactgaggaatgtcagcttcccagcctcaagatcttcatcgccgggaactcggcctacgagtacgtggactacctcttcaaacgcatgattgacctcagcagtatctccaccgtcgacagcatgatcgccctggatatcgacccgctggcgagctccyctagrtcgacatgataagatacattgatgagtttggacaaaccacaactagaatgcagtgaaaaaaatgctttatttgtgaaatttgtgatgctattgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtagatcatttaaatgttagcgaagaacatgtgagcgtgctaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctgggcttttgctggccttttgctcacatgttctt

SEQ ID NO: 17 - protein, 17 aa, cytomegalovirus
KRPLITKPEVISVMKRR

SEQ ID NO: 18 - protein, 25 aa, cytomegalovirus
KRRRHRQDALPPGPCIASTPKKHRG

SEQ ID NO: 19 - protein, 24 aa, cytomegalovirus
RKTPRVTGGGAMAGASTSAGRKRK

SEQ ID NO: 20 - protein, 26 aa, cytomegalovirus
PKRRRHRQDALPPGPCIASTPKKHRG

SEQ ID NO: 21 - DNA, 28 bp, Artificial Sequence
  PCR Primer
gtcgacggccagcatcacactagtctcc

SEQ ID NO: 22 - DNA, 28 bp, Artificial Sequence
  PCR Primer
gtcgacattgaggagatctgcatgaagg

SEQ ID NO: 23 - protein, 25 aa, Artificial Sequence
  Kinase-deficient pp65 protein containing mutation K436N
RKTPRVTGGGAMAGASTSAGRKNRK

SEQ ID NO: 24 - DNA, 22 bp, Artificial Sequence
  PCR Primer
ttgcgcagcgggctgccatacg

SEQ ID NO: 25 - DNA, 27 bp, Artificial Sequence
  PCR Primer
tgacccacgtccactcagacacgcgac

SEQ ID NO: 26 - DNA, 29 bp, Artificial Sequence
  PCR Primer
tacgaattcgacacgatgagtcctctgcc

SEQ ID NO: 27 - DNA, 29 bp, Artificial Sequence
  PCR Primer
gtgtgaggtaaaagcagccttgcttctag

SEQ ID NO: 28 - DNA, 29 bp, Artificial Sequence
  CMV-pp65 mutant II gene amplification primer
ctagaagcaaggctgcttttacctcacac

SEQ ID NO: 29 - DNA, 30 bp, Artificial Sequence
  CMV-pp65 mutant II gene reverse primer containing XbaI site
acttctagaccaaaagtcgcgtgtctgagt

SEQ ID NO: 30 - protein, 9 aa, cytomegalovirus
NLVPMVATV

SEQ ID NO: 31 - protein, 9 aa, cytomegalovirus
TMYGGISLL

SEQ ID NO: 32 - protein, 9 aa, cytomegalovirus
ILDEERDKV

SEQ ID NO: 33 - protein, 9 aa, cytomegalovirus
VLEETSVML

SEQ ID NO: 34 - DNA, 1842 bp, cytomegalovirus
cgggtcttcgtgggaggtcgggaagggttccggcaactgtcgcaaatatagcagcggcgacaggtgtggcggccaaaagtcgcgtgtctgagtggacgtgggtttttatagagtcgtcctaagcgcgtgcgcggcgggtggctcaacctcggtgctttttgggcgtcgaggcgatgcatggcccgggcaaggcgtcttgccggtggcggcgacgtttgggttgcgcagcgggctgccatacgccttccaattcggcgaagatgcggtagatgtcgttggcgtcccagaagaactcctggtacttcagattctgaccctgaaccgtagccaccatgggcaccaggttgcgggccaggatgccggcctgccagggcggccaggtgaacacggccggattgtggatttcgttgtcggaatcctcgtcggtgtcctcttcgggcgcgacggtggactcggccttaaggcggccgcgtgtcataacgcccgccgtgcacgccgtcgccgaggatgctgatttgcggttgcggcccgcggaagtggaggcgcccgccatggcgccgccgccggtaacgcggggcgtcttgcgctcggtggttacgagttcctcgtcggagtccgatccgctggtccagacgtcgtcgtcgccctgggcggcaccctcgtcgtgccggtcccaggtgtgtcggtactcaagcttgccctggatgcgatactggctggtgaaggtggggtgttcgctgtactgaggcccgcgctgcagcagcaagtcgatatcgaaaaagaagagcgcagccacgggatcgtactgacgcagttccacggtctcgcgtatcgcttgcacctccaggaagatctgctgcccgttcatcaataggttacctgagatgctcaggcccgggatgctcttgggacacagcagcccaaaatgctcgtgtgaggtaaaagccacatccagcatgatgtgcgagatcttgcccggtttgattatcatatttttgggacacaacaccgtaaagccgttgcgctcgtgggggcgcatgaagggttgcgggttgcgggtcatcgtcaggtcctcttccacgtcagagcccagcgtgacgtgcataaagagcttgccggagggcacgtcctcgcagaaggactccaggtacaccttgacgtactggtcacctatcacctgcatcttggttgcgcgcgtgttctccatggagcaaaccagctcgtgcgcgcacaccacgtgccgcagtgccacgtccttggtgggaaacacgaacgctgacgtgtagtagacgtcgggctctttccactggttctgctgacgcgtccaggccagtcccgagaccgtgagacgcgcctgccacatctgcttgcccgacgcgtgaatcacagcgtcagctacgggcaggtgtcggtgtttgcgctcggccgccgacgggtagtggtgcacgttgatgctggggatgttcagcatcttgagcggcagcgcgtacacatagatcgacatgggctcttggctggggcagatgctccggcccgtggggttgtgcacgttgaccgacacgttctccacctcgctgcccgtaaagtacgtgtgctgcacctgcagctgattgtcgccgcggtggcatggcgtcgagtcgggcgtgtactgcgacaccaggatcagcgagggctggctcacgcgcacgtggatacccgtctgcaggagtcgcgtctcgtgcggcagcaccggcgtgtcgccgcgactaaacacggctttcagcacgtgccccgaaatgggacccagtacggatatcatttcgggacaacggcgaccgcgcgactccatgctgcctgcgcgta

SEQ ID NO: 35 - DNA, 1542 bp, Cytomegalovirus
gtgacgtgggatccataacagtaactgatatatatatacaatagtttactggtcagccttgcttctagtcaccatagggtgggtgctcttgcctccagaggcggtgggttcctcagcaccatcctcctcttcctctggggcaacttcctctatctcagacactggctcagacttgacagacacagtgtcctcccgctcctcctgagcaccctcctcctcttcctcatcactctgctcactttcttcctgatcactgttctcagccacaattactgaggacagagggatagtcgcgggtacaggggactctgggggtgacaccagagaatcagaggagctgacaccagcggtggccaaagtgtaggctacaatagcctcttcctcatctgactcctcggcgatggcccgtaggtcatccacactaggagagcagactctcagaggatcggcccccagaatgtactgggcaaagaccttcatgcagatctcctcaatgcggcgcttcattacactgataacctcaggcttggttatcagaggccgcttggccagcatcacactagtctcctctaagacatagcagcacagcacccgacagaactcacttaagagagagatgcccccgtacatggtcatcatacaagcgtcactagtgaccttgtactcattacacattgtttccacacatgtagtgaggatatccataaatatgtgatcaatgtgcgtgagcaccttgtctctctcctcatccaaaatcttaaatattttctgggcataagccataatctcatcaggggagcactgaggcaagttctgcagtgccgccatggcctgactgcagccattggtggtcttagggaaggctgagttcttggtaaagaactctatattcctgtagcacatatacatcatctttctcctaagttcatcctttttagcacgggccttagcctgcagtgcaccccccaacttgttagcggcgcccttgctcacatcatgcagctccttaatacaagccatccacatctcccgcttatcctcaggtacaatgtagttctcatacatgctctgcatagttagcccaatacacttcatctcctcgaaaggctcatgaaccttatctaagatatctaaggcattctgcaaacatcctcccatcatattaaaggcgccagtgaatttctcttccgtctgggtatattttttcagcatgtgctccttgattctatgccgcaccatgtccactcgaaccttaatctgtttgacgagttctgccaggacatctttctcggggttctcgttgcaatcctcggtcacttgttcaaaagttttgagggattcttcggccaactctggaaacagcgggtctcccagactcagctgactgttaacctccttcctcaacatagtctgcaggaacgtcgtggccttggtcacgggtgtctcgggccgtggcaccttggaggaagggccctcgtcaggattatcagggtccatctttctcttggcagaggactccatcgtgtcaaggacggtgactgc

SEQ ID NO: 36 - DNA, 2347 bp, Artificial Sequence
  Fusion of the native sequences IE1 and truncated pp65mII.
ccaaaagtcgcgtgtctgagtggacgtgggtttttatagagtcgtcctaagcgcgtgcgcggcgggtggctcaacctcggtgctttttgggcgtcgaggcgatgcatggcccgggcaaggcgtcttgccggtggcggcgacgtttgggttgcgcagcgggctgccatacgccttccaattcggcgaagatgcggtagatgtcgttggcgtcccagaagaactcctggtacttcagattctgaccctgaaccgtagccaccatgggcaccaggttgcgggccaggatgccggcctgccagggcggccaggtgaacacggccggattgtggatttcgttgtcggaatcctcgtcggtgtcctcttcgggcgcgacggtggactcggccttaaggcggccgcgtgtcataacgcccgccgtgcacgccgtcgccgaggatgctgatttgcgtttgcggcccgcggaagtggaggcgcccgccatggcgccgccgccggtaacgcggggcgtcttgcgctcggtggttacgagttcctcgtcggagtccgatccgctggtccagacgtcgtcgtcgccctgggcggcaccctcgtcgtgccggtcccaggtgtgtcggtactcaagcttgccctggatgcgatactggctggtgaaggtggggtgttcgctgtactgaggcccgcgctgcagcagcaagtcgatatcgaaaaagaagagcgcagccacgggatcgtactgacgcagttccacggtctcgcgtatcgcttgcacctccaggaagatctgctgcccgttcatcaataggttacctgagatgctcaggcccgggatgctcttgggacacagcagcccaaaatgctcgtgtgaggtaaaagcagccttgcttctagtcaccatagggtgggtgctcttgcctccagaggcggtgggttcctcagcaccatcctcctcttcctctggggcaacttcctctatctcagacactggctcagacttgacagacacagtgtcctcccgctcctcctgagcaccctcctcctcttcctcatcactctgctcactttcttcctgatcactgttctcagccacaattactgaggacagagggatagtcgcgggtacaggggactctgggggtgacaccagagaatcagaggagctgacaccagcggtggccaaagtgtaggctacaatagcctcttcctcatctgactcctcggcgatggcccgtaggtcatccacactaggagagcagactctcagaggatcggcccccagaatgtactgggcaaagaccttcatgcagatctcctcaatgcggcgcttcattacactgataacctcaggcttggttatcagaggccgcttggccagcatcacactagtctcctctaagacatagcagcacagcacccgacagaactcacttaagagagagatgcccccgtacatggtcatcatacaagcgtcactagtgaccttgtactcattacacattgtttccacacatgtagtgaggatatccataaatatgtgatcaatgtgcgtgagcaccttgtctctctcctcatccaaaatcttaaatattttctgggcataagccataatctcatcaggggagcactgaggcaagttctgcagtgccgccatggcctgactgcagccattggtggtcttagggaaggctgagttcttggtaaagaactctatattcctgtagcacatatacatcatctttctcctaagttcatcctttttagcacgggccttagcctgcagtgcaccccccaacttgttagcggcgcccttgctcacatcatgcagctccttaatacaagccatccacatctcccgcttatcctcaggtacaatgtagttctcatacatgctctgcatagttagcccaatacacttcatctcctcgaaaggctcatgaaccttatctaagatatctaaggcattctgcaaacatcctcccatcatattaaaggcgccagtgaatttctcttccgtctgggtatattttttcagcatgtgctccttgattctatgccgcaccatgtccactcgaaccttaatctgtttgacgagttctgccaggacatctttctcggggttctcgttgcaatcctcggtcacttgttcaaaagttttgagggattcttcggccaactctggaaacagcgggtctcccagactcagctgactgttaacctccttcctcaacatagtctgcaggaacgtcgtggccttggtcacgggtgtctcgggccgtggcaccttggaggaagggccctcgtcaggattatcagggtccatctttctcttggcagaggactccatcgtgtc

SEQ ID NO: 37 - DNA, 9167 bp, Artificial Sequence
  Combined fusion of IE1 and pp65mII plus a truncated gB which contains the immunologic domains.
aagaacatgtgagcaaaaggccagcaaaagcccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttagcacgctcacatgttcttcgctaacatttaaatgatctaccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcaatagcatcacaaatttcacaaataaagcatttttttcactgcattctagttgtggtttgtccaaactcatcaatgtatcttatcatgtcgayctagrggagctcgccagcgggtcgatatccagggcgatcatgctgtcgacggtggagatactgctgaggtcaatcatgcgtttgaagaggtagtccacgtactcgtaggccgagttcccggcgatgaagatcttgaggctgggaagctgacattcctcagtgcggtggttgcccaacaggatttcgttgtcctcgcccagttgaccgtactgcacgtacgagctgttggcgaaattaaagatgaccacgggtcgtgagtagcagcgtcctggcgattccttcacgttcatatcacgcagcaccttgacgctggtttggttgatggtcacgcagctggccaggcccaagacatcacccatgaaacgcgcggcaatcggtttgttgtaaatggccgagagaatggctgacgggttgatcttgctgagttccttgaagacctctagggtgcgccgttgatccacacaccaggcttctgcgatttgcgccagcgcccggttgatgtaaccgcgcaacgtgtcataggtgaactgcagctgggcgtagaccagattgtgcaccgattccatgctggacaantgagttgtattattgtcactcgtacttcttctggtcctatgagtgatattcagactggatcgattggccaaacgttccaattccaccaaagatttttgcttgatgccttgccagaacaccaccagaccgccgctggtttcgaagacggacacgtttccgtatttttcatatgtttgattgtatgaagtattgaaaatctgctgtaacttatttatagcctcatcacgtacgcagtccagcgcggagtcggacatgttcacttcttgtttcttagacagaaaagttgcagtcattttggcagaagaaaagtggtacgagtcttcggcttcggaacggatagtacgttccgaggcttcccagaaggtgagctggcaggtgacattcttctcgtcctgtatatcccaagagatcaccgagtcggcacgttcgagaaaagccaccaacctatgggtttctggcgcagcgttgggtcttccaaagtcggaaacgatggtgtagttcgggaaaatgaaaaacttgtcggcgttttctccaaagtagctggcattgcgattggttccgttgtagaaaggagaaatgtaaaccacatcacccgtggaagttgcaaaaaaatgataaggatacttggagcgcgcagtagtgatggtcagcatacagttcagattacaggtctcacgatagagccaggtgctgccgcggctgtgccactgatccttgaccgtcacgtaacgggtactgtgggtgttggaataatcgtcgggaattaattgcatggttttgttttcataactgtccctatgatatgccacgaaaaccgtgcctcctataacgcggctgtaggaactgtagcattgagcaaacttgttgatgtgatgaatctcccacataggaggcgccacgtattccgtattgctgcccagcagataagtggtgtagatgtaagcgtagctacgacgaaacgtcaaaaccttttggtagacccgtaccttaaaggtgtgcgccacgatgttgcgcttgtagaccaccatgatgccctcatccaagtcttcattgataggcttcatcgaggtgcagatgatattacgttcaaagcgaataagatccgtaccctgggccatagaacacacgcgataggggtacttggtagtgttgactcccaccacatctccgtacttgagggtagtgttgtagatagtctcgttggctctatgactgacgcgggtgtctcgggcctacgtggcaccttggaggaagggccctcgtcaggattatcagggtccatctttctcttggcagaggactccatcgtgtcgaattccaccacactggactagtggatccgagctcggtaccaagcttaagtttaaacgctgcagaaaagacccatggaaaggaacagtctgttagtctgtcagctattatgtctggtggcgcgcgcggcagcaacgagtactgctcagactacactgccctccaccgttaacagcaccgcaacgggagttacctctgactcttatcagaacacaacaactcagctgcctgcatcttcttctgccgctgccttaagtcttccaaatgcgtcagcggtgcaagcccgctccccgagctcattttcagacacataccctaccgccacggccttgtgcggcacactggtggtggtgggcatcgtgctgtgcctaagtctggcctccactgttaggagcaaggagctgccgagcgaccatgagtcgctggaggcatgggagcagggctcggatgtagaagctccgccgctaccggagaagagcccatgtccggaacacgtacccgagattcgcgtggagatcccacgttatgtttaataaaaactgcgggcactggggacggtggtgttgtatatgtgaatttgtaaataataaatgagaccccatcctgtaaaaatacagagtccgtgtcagtctctgaaggacagtgtattggcatatagccaataaagagagttgtggcaaagagccatgttatggattagtaatggaaagtatcgtcaccaataggggagtggtcaataatggtcaataacccacacctataggctaagctataccatcacctataacatgaggaagcgggggtgtatagaccccaagccaaaaacagtatagcatgcataagaagccaagggggtgggcctatagactctataggcggtacttacactagccagcttgggtctccctatagtgagtcgtattaatttcgataagccagtaagcagtgggttctctagttagccagagagctctgcttatatagacctcccaccgtacacgcctaccgcccatttgcgtcaatggggcggagttgttacgacattttggaaagtcccgttgattttggtgccaaaacaaactcccattgacgtcaatggggtggagacttggaaatccccgtgagtcaaaccgctatccacgcccattgatgtactgccaaaaccgcatcaccatggtaatagcgatgactaatacgtagatgtactgccaagtaggaaagtcccataaggtcatgtactgggcataatgccaggcgggccatttaccgtcattgacgtcaatagggggcgtacttggcatatgatacacttgatgtactgccaagtgggcagtttaccgtaaatagtccacccattgacgtcaatggaaagtccctattggcgttactatgggaacatacgtcattattgacgtcaatgggcgggggtcgttgggcggtcagccaggcgggccatttaccgtaagttatgtaacgcggaactccatatatgggctatgaactaatgaccccgtaattgattactattaataactagtcaataatcaatgtcaacgcgtatatctggcccgtacatcgcgaagagtcgcttcagaagacgttacgtgttgagaatagactgaccgggtttgagcagacgtcgtacgagaagtatggcttccattgtgagtagaagaagttgcatgggaagtactagaagaggaaaccgcagcacccagacagacgatacacaggttaacgcagactaccaggcaccagatcctggattccatgttaagcttcggatccgrgatcyaggcctccttcgccggtgatctcagctgtaggaaagagaagaaggttagtagtcgacgtgtccctctcgatgaatctaagtatcaattgtgagcgctcacaagtcaacactctttttgataaatctagtagatatcacttacgtaggcgccggtcacagcttggatctgtaacggcgcagaacagaaaacgaaacaaagacgtagagttgagcaagcagggtcaggcaaagcgtggagagccggctgagtctaggtaggctccaagggagcgccggacaaaggcccggtctcgacctgagctttaaacttacctagacggcggacgcagttcaggaggcaccacaggcgggaggcggcagaacgcgactcaaccggcgtggatggcggcctcaggtagggcggcgggcgcgtgaaggagagatgcgagcccctcgaagcttcagctgtgttctggcggcaaacccgttgcgaaaaagaacgttcacggcgactactgcacttatatacggttctcccccaccctcgggaaaaaggcggagccagtacacgacatcactttcccagtttaccccgcgccaccttctctaggcaccggttcaattgccgacccctccccccaacttctcggggactgtgggcgatgtgcgctctgcccactgacgggcaccggagcgatcgcagatccttcgatagagaaatgttctggcacctgcacttggtgctattattgaagcatttatcagggttattgtctcatgagcggatacatatttgaatgtatttagaaaaataaacaaataggggttccgcgcacatttccccgaaaagtgccacctgtatgcggtgtgaaataccgcacagatgcgtaaggagaaaataccgcatcaggaaattgtaagcgttaataattcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgctcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcttgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcatcctgtctcttgatcagagcttgatcccctgcgccatcagatccttggcggcgagaaagccatccagtttactttgcagggcttcccaaccttaccagagggcgccccagctggcaattccggttcgcttgctgtccataaaaccgcccagtagaagccatagagcccaccgcatccccagcatgcctgctattgtcttcccaatcctcccccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcaactagaaggcacagtcgaggctgatcagcgggtttaaacgggccctctagaccaaaagtcgcgtgtctgagtggacgtgggtttttatagagtcgtcctaagcgcgtgcgcggcgggtggctcaacctcggtgctttttgggcgtcgaggcgatgcatggcccgggcaaggcgtcttgccggtggcggcgacgtttgggttgcgcagcgggctgccatacgccttccaattcggcgaagatgcggtagatgtcgttggcgtcccagaagaactcctggtacttcagattctgaccctgaaccgtagccaccatgggcaccaggttgcgggccaggatgccggcctgccagggcggccaggtgaacacggccggattgtggatttcgttgtcggaatcctcgtcggtgtcctcttcgggcgcgacggtggactcggccttaaggcggccgcgtgtcataacgcccgccgtgcacgccgtcgccgaggatgctgatttgcgtttgcggcccgcggaagtggaggcgcccgccatggcgccgccgccggtaacgcggggcgtcttgcgctcggtggttacgagttcctcgtcggagtccgatccgctggtccagacgtcgtcgtcgccctgggcggcaccctcgtcgtgccggtcccaggtgtgtcggtactcaagcttgccctggatgcgatactggctggtgaaggtggggtgttcgctgtactgaggcccgcgctgcagcagcaagtcgatatcgaaaaagaagagcgcagccacgggatcgtactgacgcagttccacggtctcgcgtatcgcttgcacctccaggaagatctgctgcccgttcatcaataggttacctgagatgctcaggcccgggatgctcttgggacacagcagcccaaaatgctcgtgtgaggtaaaagcagccttgcttctagtcaccatagggtgggtgctcttgcctccagaggcggtgggttcctcagcaccatcctcctcttcctctggggcaacttcctctatctcagacactggctcagacttgacagacacagtgtcctcccgctcctcctgagcaccctcctcctcttcctcatcactctgctcactttcttcctgatcactgttctcagccacaattactgaggacagagggatagtcgcgggtacaggggactctgggggtgacaccagagaatcagaggagctgacaccagcggtggccaaagtgtaggctacaatagcctcttcctcatctgactcctcggcgatggcccgtaggtcatccacactaggagagcagactctcagaggatcggcccccagaatgtactgggcaaagaccttcatgcagatctcctcaatgcggcgcttcattacactgataacctcaggcttggttatcagaggccgcttggccagcatcacactagtctcctctaagacatagcagcacagcacccgacagaactcacttaagagagagatgcccccgtacatggtcatcatacaagcgtcactagtgaccttgtactcattacacattgtttccacacatgtagtgaggatatccataaatatgtgatcaatgtgcgtgagcaccttgtctctctcctcatccaaaatcttaaatattttctgggcataagccataatctcatcaggggagcactgaggcaagttctgcagtgccgccatggcctgactgcagccattggtggtcttagggaaggctgagttcttggtaaagaactctatattcctgtagcacatatacatcatctttctcctaagttcatcctttttagcacgggccttagcctgcagtgcaccccccaacttgttagcggcgcccttgctcacatcatgcagctccttaatacaagccatccacatctcccgcttatcctcaggtacaatgtagttctcatacatgctctgcatagttagcccaatacacttcatctcctcgaaaggctcatgaaccttatctaagatatctaaggcattctgcaaacatcctcccatcatattaaaggcgccagtgaatttctcttccgtctgggtatattttttcagcatgtgctccttgattctatgccgcaccatgtccactcgaaccttaatctgtttgacgagttctgccaggacatctttctcggggttctcgttgcaatcctcggtcacttgttcaaaagttttgagggattcttcggccaactctggaaacagcgggtctcccagactcagctgactgttaacctccttcctcaacatagtctgcaggaacgtcgtggccttggtcacgggtgtctcgggctca

SEQ ID NO: 38 - DNA, 1716 bp, Artificial Sequence
  Full-length CMV pp65mII with NLS-KO
cgggtcttcgtgggaggtcgggaagggttccggcaactgtcgcaaatatagcagcggcgacaggtgtggcggccaaaagtcgcgtgtctgagtggacgtgggtcagctgccatacgccttccaattcggcgaagatgcggtagatgtcgttggcgtcccagaagaactcctggtacttcagattctgaccctgaaccgtagccaccatgggcaccaggttgcgggccaggatgccggcctgccagggcggccaggtgaacacggccggattgtggatttcgttgtcggaatcctcgtcggtgtcctcttcgggcgcgacggtggactcggccttaaggcggccgcgtgtcataacgcccgccgtgcacgccgtcgccgaggatgctgatttgcggttgcggcccgcggaagtggaggcgcccgccatggcgccgccgccggtaacgcggggcgtcttgcgctcggtggttacgagttcctcgtcggagtccgatccgctggtccagacgtcgtcgtcgccctgggcggcaccctcgtcgtgccggtcccaggtgtgtcggtactcaagcttgccctggatgcgatactggctggtgaaggtggggtgttcgctgtactgaggcccgcgctgcagcagcaagtcgatatcgaaaaagaagagcgcagccacgggatcgtactgacgcagttccacggtctcgcgtatcgcttgcacctccaggaagatctgctgcccgttcatcaataggttacctgagatgctcaggcccgggatgctcttgggacacagcagcccaaaatgctcgtgtgaggtaaaagccacatccagcatgatgtgcgagatcttgcccggtttgattatcatatttttgggacacaacaccgtaaagccgttgcgctcgtgggggcgcatgaagggttgcgggttgcgggtcatcgtcaggtcctcttccacgtcagagcccagcgtgacgtgcataaagagcttgccggagggcacgtcctcgcagaaggactccaggtacaccttgacgtactggtcacctatcacctgcatcttggttgcgcgcgtgttctccatggagcaaaccagctcgtgcgcgcacaccacgtgccgcagtgccacgtccttggtgggaaacacgaacgctgacgtgtagtagacgtcgggctctttccactggttctgctgacgcgtccaggccagtcccgagaccgtgagacgcgcctgccacatctgcttgcccgacgcgtgaatcacagcgtcagctacgggcaggtgtcggtgtttgcgctcggccgccgacgggtagtggtgcacgttgatgctggggatgttcagcatcttgagcggcagcgcgtacacatagatcgacatgggctcttggctggggcagatgctccggcccgtggggttgtgcacgttgaccgacacgttctccacctcgctgcccgtaaagtacgtgtgctgcacctgcagctgattgtcgccgcggtggcatggcgtcgagtcgggcgtgtactgcgacaccaggatcagcgagggctggctcacgcgcacgtggatacccgtctgcaggagtcgcgtctcgtgcggcagcaccggcgtgtcgccgcgactaaacacggctttcagcacgtgccccgaaatgggacccagtacggatatcatttcgggacaacggcgaccgcgcgactccatgctgcctgcgcgta

SEQ ID NO: 39 - DNA, 1503 bp, Artificial Sequence
  Full-length CMV IE1 with NLS-KO
gtgacgtgggatccataacagtaactgatatatatatacaatagtttactggtcagccttgcttctagtcaccatagggtgggtgctcttgcctccagaggcggtgggttcctcagcaccatcctcctcttcctctggggcaacttcctctatctcagacactggctcagacttgacagacacagtgtcctcccgctcctcctgagcaccctcctcctcttcctcatcactctgctcactttcttcctgatcactgttctcagccacaattactgaggacagagggatagtcgcgggtacaggggactctgggggtgacaccagagaatcagaggagctgacaccagcggtggccaaagtgtaggctacaatagcctcttcctcatctgactcctcggcgatggcccgtaggtcatccacactaggagagcagactctcagaggatcggcccccagaatgtactgggcaaagaccttcatgcagatctcctcaatgtcgacgtcgacggccagcatcacactagtctcctctaagacatagcagcacagcacccgacagaactcacttaagagagagatgcccccgtacatggtcatcatacaagcgtcactagtgaccttgtactcattacacattgtttccacacatgtagtgaggatatccataaatatgtgatcaatgtgcgtgagcaccttgtctctctcctcatccaaaatcttaaatattttctgggcataagccataatctcatcaggggagcactgaggcaagttctgcagtgccgccatggcctgactgcagccattggtggtcttagggaaggctgagttcttggtaaagaactctatattcctgtagcacatatacatcatctttctcctaagttcatcctttttagcacgggccttagcctgcagtgcaccccccaacttgttagcggcgcccttgctcacatcatgcagctccttaatacaagccatccacatctcccgcttatcctcaggtacaatgtagttctcatacatgctctgcatagttagcccaatacacttcatctcctcgaaaggctcatgaaccttatctaagatatctaaggcattctgcaaacatcctcccatcatattaaaggcgccagtgaatttctcttccgtctgggtatattttttcagcatgtgctccttgattctatgccgcaccatgtccactcgaaccttaatctgtttgacgagttctgccaggacatctttctcggggttctcgttgcaatcctcggtcacttgttcaaaagttttgagggattcttcggccaactctggaaacagcgggtctcccagactcagctgactgttaacctccttcctcaacatagtctgcaggaacgtcgtggccttggtcacgggtgtctcgggccgtggcaccttggaggaagggccctcgtcaggattatcagggtccatctttctcttggcagaggactccatcgtgtcaaggacggtgactgc

SEQ ID NO: 40 - DNA, 2160 bp, Artificial Sequence
  Fusion of IE1 and the truncated pp65mII with NLS-KO in each.
tcattgcgcagcgggctgccatacgccttccaattcggcgaagatgcggtagatgtcgttggcgtcccagaagaactcctggtacttcagattctgaccctgaaccgtagccaccatgggcaccaggttgcgggccaggatgccggcctgccagggcggccaggtgaacacggccggattgtggatttcgttgtcggaatcctcgtcggtgtcctcttcgggcgcgacggtggactcggccttaaggcggccgcgtgtcataacgcccgccgtgcacgccgtcgccgaggatgctgatttgcgtttgcggcccgcggaagtggaggcgcccgccatggcgccgccgccggtaacgcggggcgtcttgcgctcggtggttacgagttcctcgtcggagtccgatccgctggtccagacgtcgtcgtcgccctgggcggcaccctcgtcgtgccggtcccaggtgtgtcggtactcaagcttgccctggatgcgatactggctggtgaaggtggggtgttcgctgtactgaggcccgcgctgcagcagcaagtcgatatcgaaaaagaagagcgcagccacgggatcgtactgacgcagttccacggtctcgcgtatcgcttgcacctccaggaagatctgctgcccgttcatcaataggttacctgagatgctcaggcccgggatgctcttgggacacagcagcccaaaatgctcgtgtgaggtaaaagcagccttgcttctagtcaccatagggtgggtgctcttgcctccagaggcggtgggttcctcagcaccatcctcctcttcctctggggcaacttcctctatctcagacactggctcagacttgacagacacagtgtcctcccgctcctcctgagcaccctcctcctcttcctcatcactctgctcactttcttcctgatcactgttctcagccacaattactgaggacagagggatagtcgcgggtacaggggactctgggggtgacaccagagaatcagaggagctgacaccagcggtggccaaagtgtaggctacaatagcctcttcctcatctgactcctcggcgatggcccgtaggtcatccacactaggagagcagactctcagaggatcggcccccagaatgtactgggcaaagaccttcatgcagatctcctcaatgacgtcgacggccagcatcacactagtctcctctaagacatagcagcacagcacccgacagaactcacttaagagagagatgcccccgtacatggtcatcatacaagcgtcactagtgaccttgtactcattacacattgtttccacacatgtagtgaggatatccataaatatgtgatcaatgtgcgtgagcaccttgtctctctcctcatccaaaatcttaaatattttctgggcataagccataatctcatcaggggagcactgaggcaagttctgcagtgccgccatggcctgactgcagccattggtggtcttagggaaggctgagttcttggtaaagaactctatattcctgtagcacatatacatcatctttctcctaagttcatcctttttagcacgggccttagcctgcagtgcaccccccaacttgttagcggcgcccttgctcacatcatgcagctccttaatacaagccatccacatctcccgcttatcctcaggtacaatgtagttctcatacatgctctgcatagttagcccaatacacttcatctcctcgaaaggctcatgaaccttatctaagatatctaaggcattctgcaaacatcctcccatcatattaaaggcgccagtgaatttctcttccgtctgggtatattttttcagcatgtgctccttgattctatgccgcaccatgtccactcgaaccttaatctgtttgacgagttctgccaggacatctttctcggggttctcgttgcaatcctcggtcacttgttcaaaagttttgagggattcttcggccaactctggaaacagcgggtctcccagactcagctgactgttaacctccttcctcaacatagtctgcaggaacgtcgtggccttggtcacgggtgtctcgggccgtggcaccttggaggaagggccctcgtcaggattatcagggtccatctttctcttggcagaggactccatcgtgtc

SEQ ID NO: 41 - DNA, 9033 bp, Artificial Sequence
  Fusion of IE1 and the truncated pp65mII with NLS-KO in each, plus a truncated gB which contains the immunologic domains.
aagaacatgtgagcaaaaggccagcaaaagcccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttagcacgctcacatgttcttcgctaacatttaaatgatctaccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcaatagcatcacaaatttcacaaataaagcatttttttcactgcattctagttgtggtttgtccaaactcatcaatgtatcttatcatgtcgayctagrggagctcgccagcgggtcgatatccagggcgatcatgctgtcgacggtggagatactgctgaggtcaatcatgcgtttgaagaggtagtccacgtactcgtaggccgagttcccggcgatgaagatcttgaggctgggaagctgacattcctcagtgcggtggttgcccaacaggatttcgttgtcctcgcccagttgaccgtactgcacgtacgagctgttggcgaaattaaagatgaccacgggtcgtgagtagcagcgtcctggcgattccttcacgttcatatcacgcagcaccttgacgctggtttggttgatggtcacgcagctggccaggcccaagacatcacccatgaaacgcgcggcaatcggtttgttgtaaatggccgagagaatggctgacgggttgatcttgctgagttccttgaagacctctagggtgcgccgttgatccacacaccaggcttctgcgatttgcgccagcgcccggttgatgtaaccgcgcaacgtgtcataggtgaactgcagctgggcgtagaccagattgtgcaccgattccatgctggacaantgagttgtattattgtcactcgtacttcttctggtcctatgagtgatattcagactggatcgattggccaaacgttccaattccaccaaagatttttgcttgatgccttgccagaacaccaccagaccgccgctggtttcgaagacggacacgtttccgtatttttcatatgtttgattgtatgaagtattgaaaatctgctgtaacttatttatagcctcatcacgtacgcagtccagcgcggagtcggacatgttcacttcttgtttcttagacagaaaagttgcagtcattttggcagaagaaaagtggtacgagtcttcggcttcggaacggatagtacgttccgaggcttcccagaaggtgagctggcaggtgacattcttctcgtcctgtatatcccaagagatcaccgagtcggcacgttcgagaaaagccaccaacctatgggtttctggcgcagcgttgggtcttccaaagtcggaaacgatggtgtagttcgggaaaatgaaaaacttgtcggcgttttctccaaagtagctggcattgcgattggttccgttgtagaaaggagaaatgtaaaccacatcacccgtggaagttgcaaaaaaatgataaggatacttggagcgcgcagtagtgatggtcagcatacagttcagattacaggtctcacgatagagccaggtgctgccgcggctgtgccactgatccttgaccgtcacgtaacgggtactgtgggtgttggaataatcgtcgggaattaattgcatggttttgttttcataactgtccctatgatatgccacgaaaaccgtgcctcctataacgcggctgtaggaactgtagcattgagcaaacttgttgatgtgatgaatctcccacataggaggcgccacgtattccgtattgctgcccagcagataagtggtgtagatgtaagcgtagctacgacgaaacgtcaaaaccttttggtagacccgtaccttaaaggtgtgcgccacgatgttgcgcttgtagaccaccatgatgccctcatccaagtcttcattgataggcttcatcgaggtgcagatgatattacgttcaaagcgaataagatccgtaccctgggccatagaacacacgcgataggggtacttggtagtgttgactcccaccacatctccgtacttgagggtagtgttgtagatagtctcgttggctctatgactgacggcttcagaagacgttacgtgttgagaatagactgaccgggtttgagcagacgtcgtacgagaagtatggcttccattgtgagtagaagaagttgcatgggaagtactagaagaggaaaccgcagcacccagacagacgatacacaggttaacgcagactaccaggcaccagatcctggattccatgttaagcttcggatccgrgatcyaggcctccttcgccggtgatctcagctgtaggaaagagaagaaggttagtagtcgacgtgtccctctcgatgaatctaagtatcaattgtgagcgctcacaagtcaacactctttttgataaatctagtagatatcacttacgtaggcgccggtcacagcttggatctgtaacggcgcagaacagaaaacgaaacaaagacgtagagttgagcaagcagggtcaggcaaagcgtggagagccggctgagtctaggtaggctccaagggagcgccggacaaaggcccggtctcgacctgagctttaaacttacctagacggcggacgcagttcaggaggcaccacaggcgggaggcggcagaacgcgactcaaccggcgtggatggcggcctcaggtagggcggcgggcgcgtgaaggagagatgcgagcccctcgaagcttcagctgtgttctggcggcaaacccgttgcgaaaaagaacgttcacggcgactactgcacttatatacggttctcccccaccctcgggaaaaaggcggagccagtacacgacatcactttcccagtttaccccgcgccaccttctctaggcaccggttcaattgccgacccctccccccaacttctcggggactgtgggcgatgtgcgctctgcccactgacgggcaccggagcgatcgcagatccttcgatagagaaatgttctggcacctgcacttggtgctattattgaagcatttatcagggttattgtctcatgagcggatacatatttgaatgtatttagaaaaataaacaaataggggttccgcgcacatttccccgaaaagtgccacctgtatgcggtgtgaaataccgcacagatgcgtaaggagaaaataccgcatcaggaaattgtaagcgttaataattcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgctcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcttgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcatcctgtctcttgatcagagcttgatcccctgcgccatcagatccttggcggcgagaaagccatccagtttactttgcagggcttcccaaccttaccagagggcgccccagctggcaattccggttcgcttgctgtccataaaaccgcccagtagaagccatagagcccaccgcatccccagcatgcctgctattgtcttcccaatcctcccccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcaactagaaggcacagtcgaggctgatcagcgggtttaaacgggccctctagaccaaaagtcgcgtgtctgagtggacgtgggtttttatagagtcgtcctaagcgcgtgcgcggcgggtggctcaaccgctgccatacgccttccaattcggcgaagatgcggtagatgtcgttggcgtcccagaagaactcctggtacttcagattctgaccctgaaccgtagccaccatgggcaccaggttgcgggccaggatgccggcctgccagggcggccaggtgaacacggccggattgtggatttcgttgtcggaatcctcgtcggtgtcctcttcgggcgcgacggtggactcggccttaaggcggccgcgtgtcataacgcccgccgtgcacgccgtcgccgaggatgctgatttgcgtttgcggcccgcggaagtggaggcgcccgccatggcgccgccgccggtaacgcggggcgtcttgcgctcggtggttacgagttcctcgtcggagtccgatccgctggtccagacgtcgtcgtcgccctgggcggcaccctcgtcgtgccggtcccaggtgtgtcggtactcaagcttgccctggatgcgatactggctggtgaaggtggggtgttcgctgtactgaggcccgcgctgcagcagcaagtcgatatcgaaaaagaagagcgcagccacgggatcgtactgacgcagttccacggtctcgcgtatcgcttgcacctccaggaagatctgctgcccgttcatcaataggttacctgagatgctcaggcccgggatgctcttgggacacagcagcccaaaatgctcgtgtgaggtaaaagcagccttgcttctagtcaccatagggtgggtgctcttgcctccagaggcggtgggttcctcagcaccatcctcctcttcctctggggcaacttcctctatctcagacactggctcagacttgacagacacagtgtcctcccgctcctcctgagcaccctcctcctcttcctcatcactctgctcactttcttcctgatcactgttctcagccacaattactgaggacagagggatagtcgcgggtacaggggactctgggggtgacaccagagaatcagaggagctgacaccagcggtggccaaagtgtaggctacaatagcctcttcctcatctgactcctcggcgatggcccgtaggtcatccacactaggagagcagactctcagaggatcggcccccagaatgtactgggcaaagaccttcatgcagatctcctcaatggccagcatcacactagtctcctctaagacatagcagcacagcacccgacagaactcacttaagagagagatgcccccgtacatggtcatcatacaagcgtcactagtgaccttgtactcattacacattgtttccacacatgtagtgaggatatccataaatatgtgatcaatgtgcgtgagcaccttgtctctctcctcatccaaaatcttaaatattttctgggcataagccataatctcatcaggggagcactgaggcaagttctgcagtgccgccatggcctgactgcagccattggtggtcttagggaaggctgagttcttggtaaagaactctatattcctgtagcacatatacatcatctttctcctaagttcatcctttttagcacgggccttagcctgcagtgcaccccccaacttgttagcggcgcccttgctcacatcatgcagctccttaatacaagccatccacatctcccgcttatcctcaggtacaatgtagttctcatacatgctctgcatagttagcccaatacacttcatctcctcgaaaggctcatgaaccttatctaagatatctaaggcattctgcaaacatcctcccatcatattaaaggcgccagtgaatttctcttccgtctgggtatattttttcagcatgtgctccttgattctatgccgcaccatgtccactcgaaccttaatctgtttgacgagttctgccaggacatctttctcggggttctcgttgcaatcctcggtcacttgttcaaaagttttgagggattcttcggccaactctggaaacagcgggtctcccagactcagctgactgttaacctccttcctcaacatagtctgcaggaacgtcgtggccttggtcacgggtgtctcgggctcacgggtgtctcgggcctacgtggcaccttggaggaagggccctcgtcaggattatcagggtccatctttctcttggcagaggactccatcgtgtcgaattccaccacactggactagtggatccgagctcggtaccaagcttaagtttaaacgctgcagaaaagacccatggaaaggaacagtctgttagtctgtcagctattatgtctggtggcgcgcgcggcagcaacgagtactgctcagactacactgccctccaccgttaacagcaccgcaacgggagttacctctgactcttatcagaacacaacaactcagctgcctgcatcttcttctgccgctgccttaagtcttccaaatgcgtcagcggtgcaagcccgctccccgagctcattttcagacacataccctaccgccacggccttgtgcggcacactggtggtggtgggcatcgtgctgtgcctaagtctggcctccactgttaggagcaaggagctgccgagcgaccatgagtcgctggaggcatgggagcagggctcggatgtagaagctccgccgctaccggagaagagcccatgtccggaacacgtacccgagattcgcgtggagatcccacgttatgtttaataaaaactgcgggcactggggacggtggtgttgtatatgtgaatttgtaaataataaatgagaccccatcctgtaaaaatacagagtccgtgtcagtctctgaaggacagtgtattggcatatagccaataaagagagttgtggcaaagagccatgttatggattagtaatggaaagtatcgtcaccaataggggagtggtcaataatggtcaataacccacacctataggctaagctataccatcacctataacatgaggaagcgggggtgtatagaccccaagccaaaaacagtatagcatgcataagaagccaagggggtgggcctatagactctataggcggtacttacactagccagcttgggtctccctatagtgagtcgtattaatttcgataagccagtaagcagtgggttctctagttagccagagagctctgcttatatagacctcccaccgtacacgcctaccgcccatttgcgtcaatggggcggagttgttacgacattttggaaagtcccgttgattttggtgccaaaacaaactcccattgacgtcaatggggtggagacttggaaatccccgtgagtcaaaccgctatccacgcccattgatgtactgccaaaaccgcatcaccatggtaatagcgatgactaatacgtagatgtactgccaagtaggaaagtcccataaggtcatgtactgggcataatgccaggcgggccatttaccgtcattgacgtcaatagggggcgtacttggcatatgatacacttgatgtactgccaagtgggcagtttaccgtaaatagtccacccattgacgtcaatggaaagtccctattggcgttactatgggaacatacgtcattattgacgtcaatgggcgggggtcgttgggcggtcagccaggcgggccatttaccgtaagttatgtaacgcggaactccatatatgggctatgaactaatgaccccgtaattgattactattaataactagtcaataatcaatgtcaacgcgtatatctggcccgtacatcgcgaagagtc

(19) United States
(12) Patent Application Publication
(10) Pub. No.: US 2008/0138354 A1
(43) Pub. Date: Jun. 12, 2008

(54) Cytomegalovirus vaccine

(75) Inventors: John A. Zaia, Arcadia, CA (US); Ghislaine Gallez-Hawkins, La Verne, CA (US)

Correspondence Address: ROTHWELL, FIGG, ERNST & MANBECK, P.C., 1425 K STREET, N.W., SUITE 800, WASHINGTON, DC, 20005, US

(73) Assignee: City of Hope, Duarte, CA (US)

(21) Appl. No.: 11/878,307

(22) Filed: Jul. 23, 2007

Related U.S. Application Data
(60) Provisional application No. 60/832,139, filed on Jul. 21, 2006.

(51) Int. Cl. A61K 39/00; C07H 21/00; C12N 1/00; C12Q 1/70; C12N 5/00; C07K 2/00

(52) U.S. Cl. 424/184.1; 536/23.1; 530/300; 435/243; 435/440; 435/5; 435/375

(57) ABSTRACT

This specification discloses a Cytomegalovirus vaccine which employs nuclear localization signal knock-out to improve vaccine immunogenicity and antigen presentation in a combined DNA and recombinant adeno-associated viral vector vaccination (rAAV) system.

### CROSS-REFERENCE TO RELATED APPLICATIONS

This application claims benefit of U.S. Provisional Patent Application No. 60/832,139, filed Jul. 21, 2006, which is hereby incorporated by reference in its entirety into the present application.

### STATEMENT REGARDING FEDERALLY SPONSORED RESEARCH

This invention was made with government support in the form of grant no. A1058148 from the United States Department of Health, National Institutes of Health. The United States government may have certain rights in the invention.

### BACKGROUND OF THE INVENTION

The goal of vaccine development is efficient and safe immunogenicity. The use of DNA expressing specific proteins important to elicit immune response, whether as plasmid DNA or in recombinant viral vectors, has been used widely, and a primary concern is the lack of stimulation of a robust immune response. To date, there is no potent vaccine to cytomegalovirus (CMV).

Attenuated strains of CMV such as the Towne strain do not protect against infection. The use of DNA expressing specific proteins important to elicit immune response has been used widely and the use of viral vectors such as rAAV has boosted the immune response in animals. Human cytomegalovirus pp 65 and immediate early 1 (IE1) proteins both are major immune targets that elicit immune responses in humans during natural infection with CMV. The CMV-pp 65 protein has been used to make a safer vaccine by constructing a kinase-deficient pp 65 having a point mutation at the K436N site.

In addition, a prime-boost vaccine strategy using intramuscular injection of naked DNA followed by a rAAV expressing the same CMV gene induces an immune response to CMV-pp 65 and CMV-IE1 in transgenic HLA A*0201 mice. A low titer of rAAV (10³ to 10⁴ pfu/dose) was sufficient to mount a robust immune response using this strategy. See Gallez-Hawkins G. et al, Vaccine, 2004; 23, 819-826, the disclosures of which are hereby incorporated by reference. However, the rAAV is only able to encapsidate up to 5 kb of inserted gene, making it difficult to target multiple CMV genes.

### SUMMARY OF THE INVENTION

Embodiments of the invention include an isolated nucleic acid that encodes an immunogenic peptide sequence wherein the native sequence of the peptide contains a nuclear localization signal (NLS), and wherein the nucleic acids encoding the NLS have been deleted or mutated. Preferred nucleic acids encode an immunogenic peptide sequence from human cytomegalovirus, for example the cytomegalovirus proteins pp 65, pp 65mII, IE1 or a fusion of pp 65 and IE1. Preferably, the nucleotides encoding amino acids 537-561 of pp 65 or those encoding amino acids 326-342 of IE1 are deleted, whether the nucleic acid encodes a single protein or fragment thereof or a fusion of proteins or fragments thereof.

Additional embodiments of the invention include polypeptides encoded by any of the nucleic acids disclosed herein. Preferably, the immunogenic peptides and nucleic acids are viral sequences.

Further embodiments of the invention encompass methods for vaccinating a subject in need thereof which comprises administering to said subject any of the nucleic acids or polypeptides disclosed here.

Further embodiments of the invention include methods for diagnosis of prior infection with a virus in a subject which comprises obtaining a sample from said subject that comprises lymphocytes of said subject; contacting said sample in vitro with a viral peptide as discussed herein and determining whether said polypeptide stimulates said lymphocytes, wherein stimulation indicates prior infection with said virus and wherein said immunogenic viral peptide sequence of said virus to be diagnosed. Additional methods encompassed by the invention include methods of producing virus-specific target cells for diagnosis of prior infection with a virus or determination of virus cytotoxic lymphocyte function, which comprises contacting targets cells with a viral polypeptide, discussed above, methods of using these target cells in diagnostic methods such as diagnosis of prior infection with a virus, determination of virus-specific cytotoxic lymphocytes function, and detection of virus-specific antibodies, methods of expanding T cells specific for an antigen, which comprises contacting said T cells in vitro with a viral polypeptide as discussed herein, improved methods for making a recombinant vaccine that encodes a viral target polypeptide where the native polypeptide localizes to the nucleus, which comprise knocking out the nuclear localization signals of the viral target polypeptide, and methods of improving protein expression of a recombinant protein that contains a NLS, which comprise knocking out the NLS.

### BRIEF DESCRIPTION OF THE DRAWINGS

FIG. 1 provides the nucleotide sequence of CMV pp 65mII (SEQ ID NO:1).

FIG. 2 provides the nucleotide sequence of CMV IE (N; native) (SEQ ID NO:2).

FIGS. 3 (3A and 3B) provides the nucleotide sequence of CMV IEpp65mII, the fusion of IE (N) and pp 65 (K436N, double stranded) (SEQ ID NO:3).

FIG. 4 provides the nucleotide sequence of CMV IEpp65mII (single-stranded; SEQ ID NO:4).

FIG. 5 provides the protein sequence of IEpp65mII (SEQ ID NO:5).

FIG. 6 provides the nucleotide sequence of CMV IEpp65mII-gB (SEQ ID NO:6). IEpp65 is at base pairs 1581-3954; gB is located at base pairs 6092-8095. The plasmid sequence is pVAX1 with Ef1 alpha promoter-gB-SV40A cassette added into the BbrP1 site (bp 2211).

FIG. 7 provides the nucleotide sequence of CMV pp 65mII-NLS-KO (̂pp 65mII), double-stranded (SEQ ID NO:7).

FIG. 8 provides the nucleotide sequence of CMV pp 65mII-NLS-KO (̂pp 65mII), single-stranded (SEQ ID NO:8).

FIG. 9 provides the protein sequence of CMV pp 65mII-NLS-KO (̂pp 65mII), (SEQ ID NO:9).

FIGS. 10 (10A and 10B) provides the nucleotide sequence of CMV IE-NLS-KO (̂IE), double-stranded (SEQ ID NO:10).

FIG. 11 provides the nucleotide sequence of CMV IE-NLS-KO (̂IE), single-stranded (SEQ ID NO:11).

FIG. 12 provides the protein sequence of IE-NLS-KO (̂IE), (SEQ ID NO:12).

FIGS. 13 (13A and 13B) provides the nucleotide sequence of CMV IEpp65mII-NlS-KO (̂IÊpp 65mII), double-stranded (SEQ ID NO:13).

FIG. 14 provides the nucleotide sequence of CMV IEpp65mII-NlS-KO (̂IÊpp 65mII), single-stranded (SEQ ID NO:14).

FIG. 15 provides the protein sequence of CMV IEpp65mII-NlS-KO (̂IÊpp 65mII) (SEQ ID NO:15).

FIG. 16 provides the nucleotide sequence of CMV IEpp65mII-gB-NLS-KO (SEQ ID NO:16). The sequence is in pVAX1, inserted at the following sites: ̂IÊpp 65mII: 1587-3820 bp; gB: 5958-7961 bp.

FIG. 17 is a photograph of HeLa cells transfected with pVAXintIE (17A), expressing the native IE sequence, and pVAXint̂IE (17B), expressing the NLS-KO IE sequence, stained with IE1 mAb. IE1 is shown in green and DAPI is shown in blue.

FIG. 18 is a photograph of HeLa cells transfected with pVAXintpp65mII (18A), expressing the native (K436N) pp 65 sequence and pVAXint̂pp 65mII (18B), expressing the NLS-KO (K436N) pp 65 sequence, stained with pp 65mII Mab. pp 65mII is shown in aqua/green and DAPI is shown in deep blue.

FIG. 19 is a diagram showing the minimal IE1-pp 65 fusion construct including the binding sites for EcoRI-IE1 (11A) and XbaI-pp 65 (11B) primers.

FIG. 20 is a photograph of a polyacrylamide gel showing the purity of the following proteins: lane 2, IE1pp 65mII (2362 bp); lane 3, IE1 (1488 bp); lane 4, carboxy terminal pp 65mII (874 bp). Lane 1 is empty.

FIG. 21 is a map of the CMV IE1pp 65mII and its insertion site in pVAX1.

FIG. 22 is an electrophoresis gel of HT1080 cell extracts from cells transduced with rAAV expressing the pp 65mII, ̂pp 65mII (NLS-KO), IEpp65mII and ̂IÊpp 65mII (NLS-KO) proteins as indicated, stained with anti-pp 65 monoclonal antibodies. An asterisk marks specifically stained bands.

FIG. 23 is a bar graph showing effect of in vitro NLS-KO (NLS-KO is indicated by ̂) on antigen presentation measured as percent specific ⁵¹Cr release (lysis).

FIG. 24 presents data of a chromium release assay showing target cell killing by CTL generated in vivo to CMV-IE1 in individual mice vaccinated with the native or the NLS-KO IE-pp 65mII fusion DNA.

FIG. 25 presents data of a chromium release assay showing target cell killing by CTL generated in vivo to CMV-pp 65mII in mice vaccinated with the native or the NLS-KO IE-pp 65mII fusion DNA.

FIG. 26 is a summary of the data presented in FIGS. 24 and 25 comparing the mean immune response to IE1 and pp 65 CMV proteins.

FIG. 27 presents the results of tetramer reagent binding assays.

### DESCRIPTION OF THE INVENTION

Improvements in the immonogenicity of recombinant vaccines are possible by using a nuclear localization signal knock-out of the encoded immunogenic protein/peptide. Immunogenicity is the ability to produce an immune response, therefore an immunogenic peptide, as used in this application, refers to a peptide that has the ability to elicit an immune response, such as stimulation of T cells, antibody production, and so on, as understood by those of skill in the art. This technology may be applied to any recombinant vaccine that encodes a protein or polypeptide that localizes to the nucleus, i.e. contains or encodes a nuclear localization signal. Without wishing to be bound by theory, it is believed that removal of the nuclear localization signal or mutation of the nuclear localization signal which makes it unoperable or reduces its function increases the amount of the expressed sequence in the cytoplasm and therefore improves processing and/or presentation of the antigen, and thus improves immunogenicity. Nuclear localization signals (NLS) contribute to protein trafficking within cells and thereby alter available protein in the cytoplasm, where antigen processing occurs. The nuclear localization signal of a protein is composed of two short stretches of basic residues separated by 10 or more non-conserved residues and can be identified by PSORT (a publicly available program which locates the NOS in protein sequences, as described in Geoch, Virus Res. 3:271, 1985, the disclosures of which are hereby incorporated by reference.

It has been discovered here that removal or mutation of the NLS can increase antigen processing. Knock-out of the NLS also can improve expression of the protein, such as a recombinant protein, to improve recovery of expressed protein. Therefore, embodiments of the invention take advantage of this phenomenon by removing or mutating the NLS in CMV-IE1 and in CMV-pp 65 to enhance peptide antigen presentation. Constructs with non-functional or missing NLS are termed “NLS-KO.” In general, the NLS can be rendered non-functional by deleting the nucleic acid sequence or by mutating these nucleotides to result in a non-functional protein. As used in this specification, the terms “deleted or mutated” with respect to an NLS-deleted are intended to refer to an NLS nucleotide sequence that has been changed in such a manner as to result in an NLS gene product which has considerably attenuated function with respect to nuclear localization such that the gene product exhibits a significantly higher concentration outside the nucleus compared to the gene product of an un-deleted or un-mutated sequence.

Experiments have shown that an antigen consisting of DNA encoding cytomegalovirus protein IE1 (CMV-IE1) fused to the 261 amino acid carboxy terminal of cytomegalovirus protein pp 65 (CMV-pp 65) is expressed after inoculation in mice and is able to elicit cytotoxic T lymphocytes that recognize and lyse cells presenting either peptide antigen. After inoculation with DNA encoding either antigen alone, with nuclear localization signal knock-out, or a fusion of both antigens, with nuclear localization knock out, the expression products were found more readily in the cytoplasm. The knock-out DNA constructs were as effective as the native proteins in inducing CTL responses in HHD-A2 transgenic mice, which are HHDII transgenic for HLA A*0201. Binding assays using tetramer reagents after stimulation with autologous blasts loaded with the appropriate peptides showed that the response to the fusion knock-out construct can be directed to either of the encoded proteins.

The methods of the invention provide an improvement to vaccines for cytomegalovirus which can be extended to any vaccine protein or peptide having a nuclear localization signal. The methods also can be used to generate in vitro targets that present antigen more efficiently. These targets are useful for diagnosis of specific cytotoxic lymphocyte (immune) function, for example against cytomegalovirus or the other proteins listed above. Improved antigen-based T cell expansion in vitro also is achieved using the inventive methods. In addition, a nuclear localization signal knock-out strategy can be used in any recombinant protein production method, providing that the protein contains a nuclear localization signal, to make expression and retrieval of the protein more efficient.

The nuclear localization signals (NLS) are located at amino acids 326-342 (KRPLITKPEVISVMKRR; SEQ ID NO:17) on exon 4 of CMV-IE1 and at amino acids 537-561 (KRRRHRQDALPPGPCIASTPKKHRG; SEQ ID NO:18) at the carboxy end of the CMV-pp 65 protein. The NLS in pp 65 has been reported in Schmolke et al., J. Virol. 69:1071-1078, 1995, as two motifs. Motif A-B is located at 415aa to 438aa (RKTPRVTGGGAMAGASTSAGRKRK; SEQ ID NO:19) and contained the K436N mutation. Motif C-D is located at 536aa to 561aa (PKRRRHRQDALPPGPCIASTPKKHRG; SEQ ID NO:20). These were removed from or mutated in CMV-IE1 and CMV-pp 65.

DNA constructs were created to encode the native sequences of cytomegalovirus protein IE1 and the carboxy terminal (amino acids 300-561) of cytomegalovirus protein pp 65, as well as an IE1-pp 65 fusion DNA construct that contains the full CMV-IE1 gene in frame with DNA encoding the carboxy terminal amino acids (amino acids 300-561) of the CMV pp 65 gene. These same constructs also were produced with NLS knock out. CMV vaccines were expressed from these constructs and were named as follows.

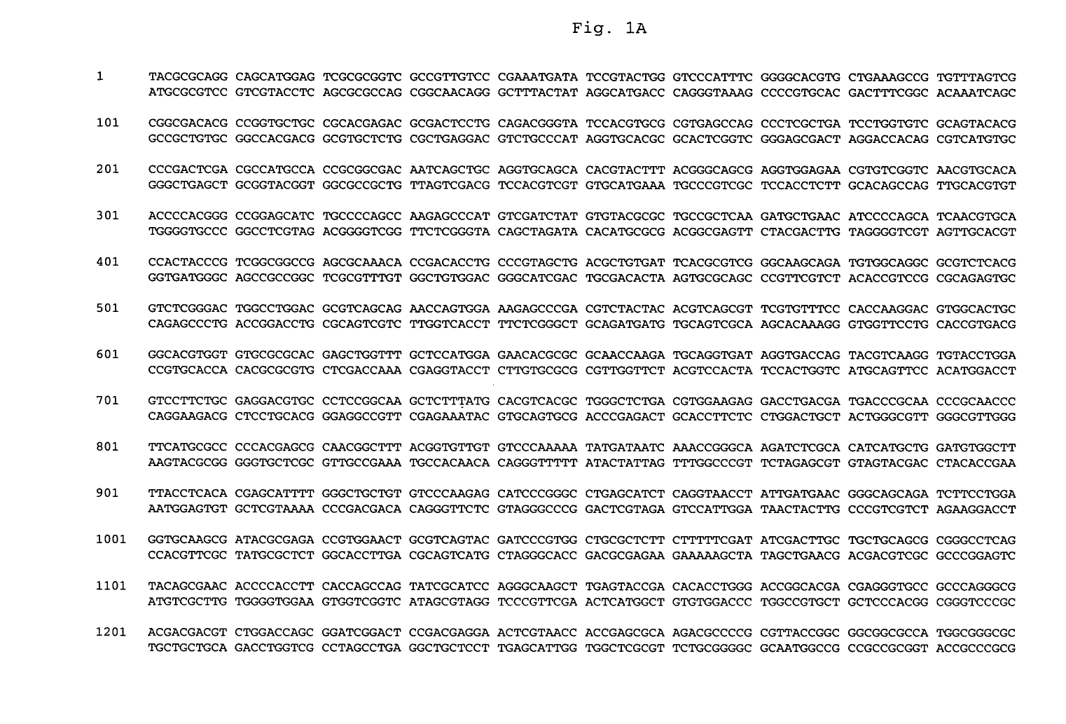

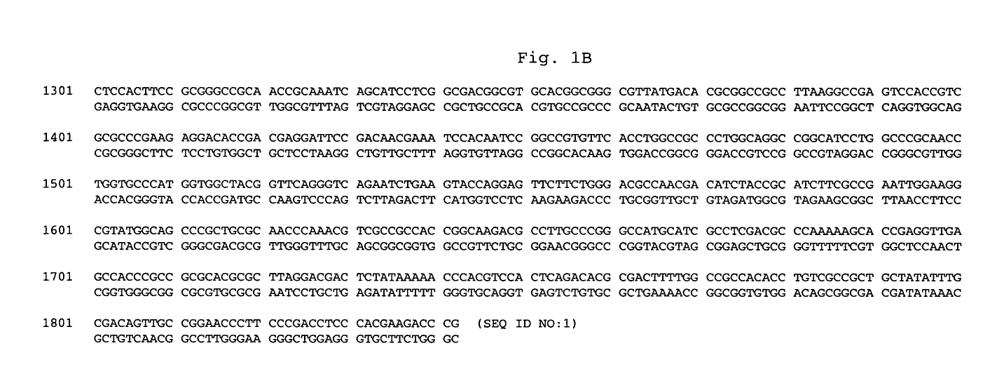

1. CMV-pp 65mII, which is a full length CMV pp 65 (native sequence) with a mutation in the phosphate binding site (K436N mutation as described in Yao et al., Vaccine 19(13-14): 1628-1635, 2001), the disclosures of which are hereby incorporated by reference. See FIG. 1 (SEQ ID NO:1).

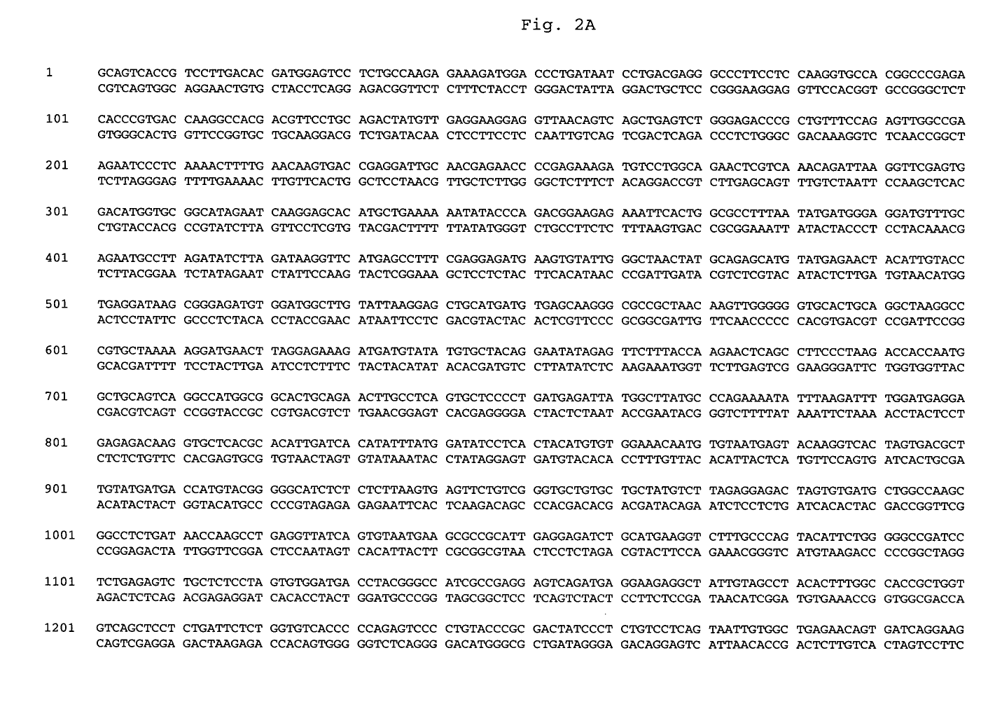

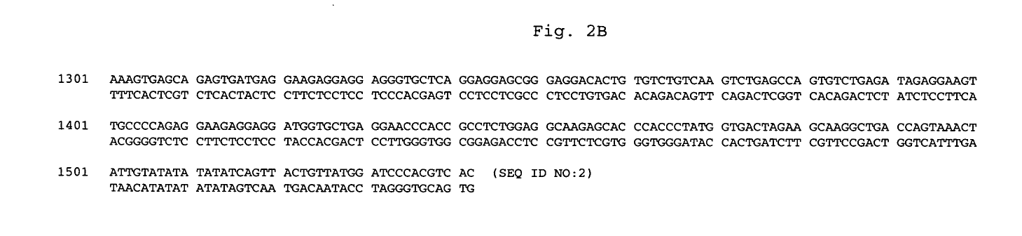

2. CMV-IE, which is a full length CMV IE1 (native sequence). See FIG. 2 (SEQ ID NO:2).

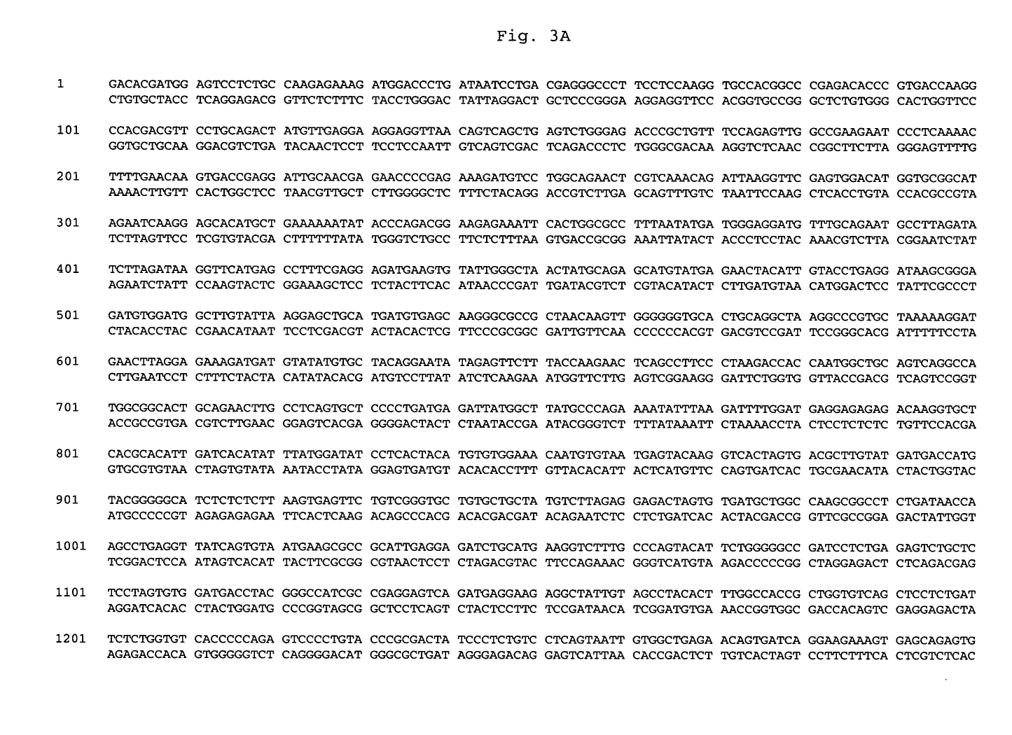

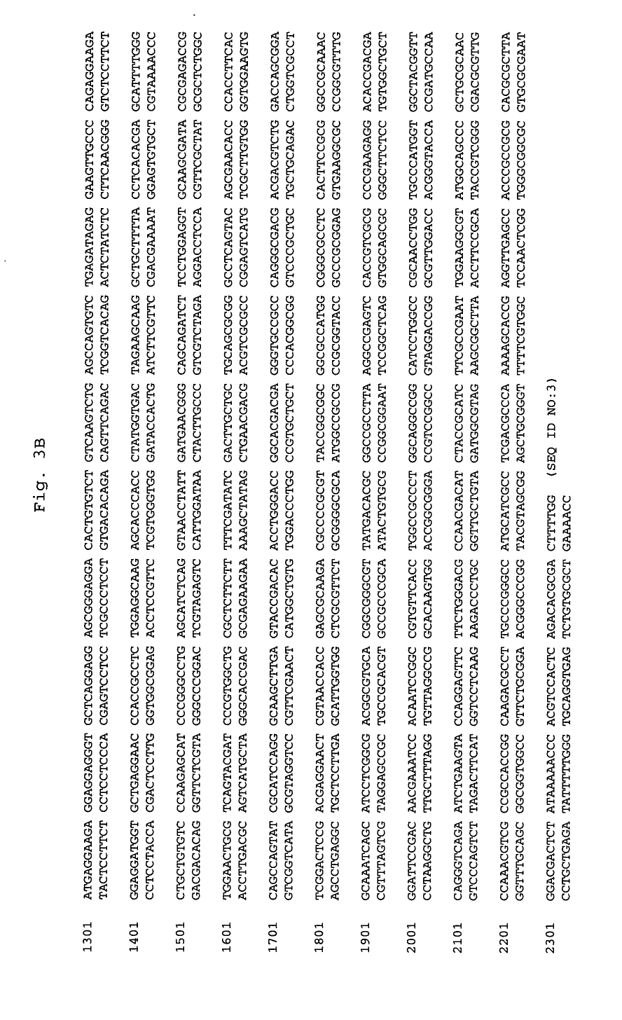

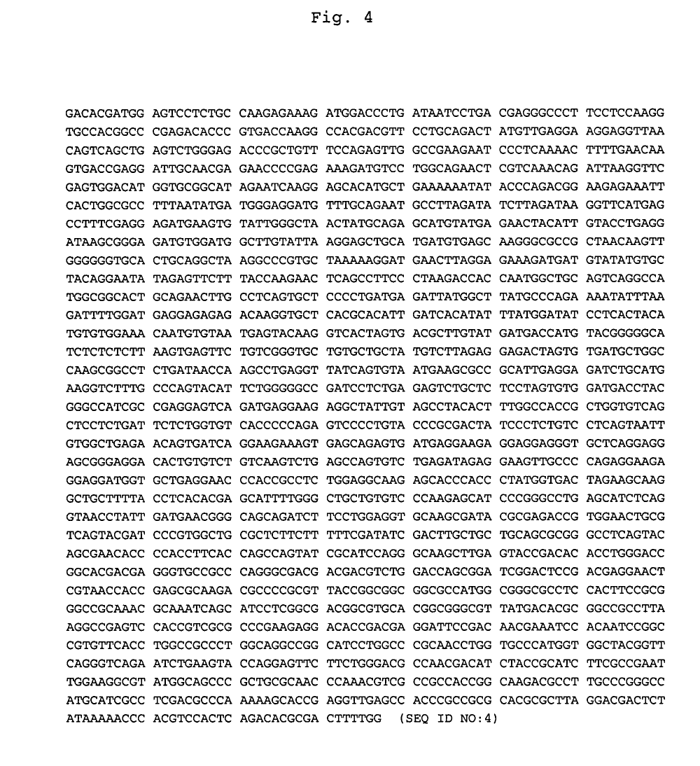

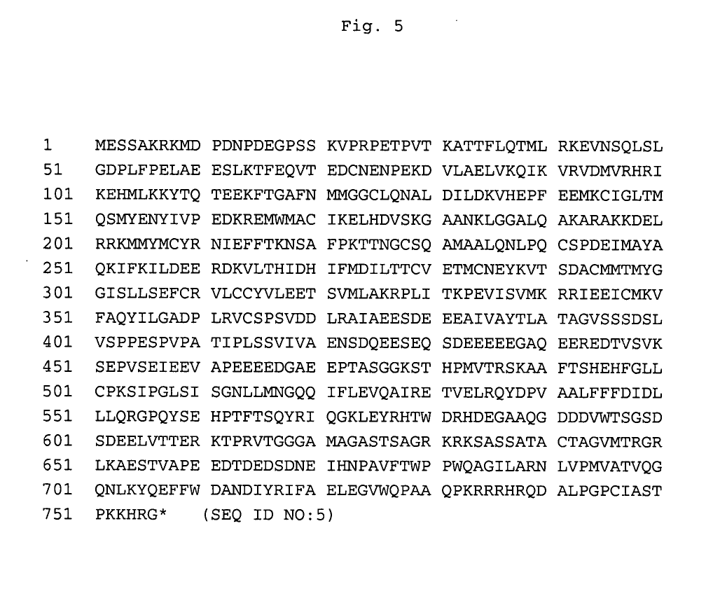

3. CMV-IEpp65mII, which is a fusion of the native sequences IE1 and truncated pp 65mII as described above. See FIGS. 3A and 3B (SEQ ID NO:3) and FIG. 4 (SEQ ID NO:4). The protein sequence is provided in FIG. 5 (SEQ ID NO:5).

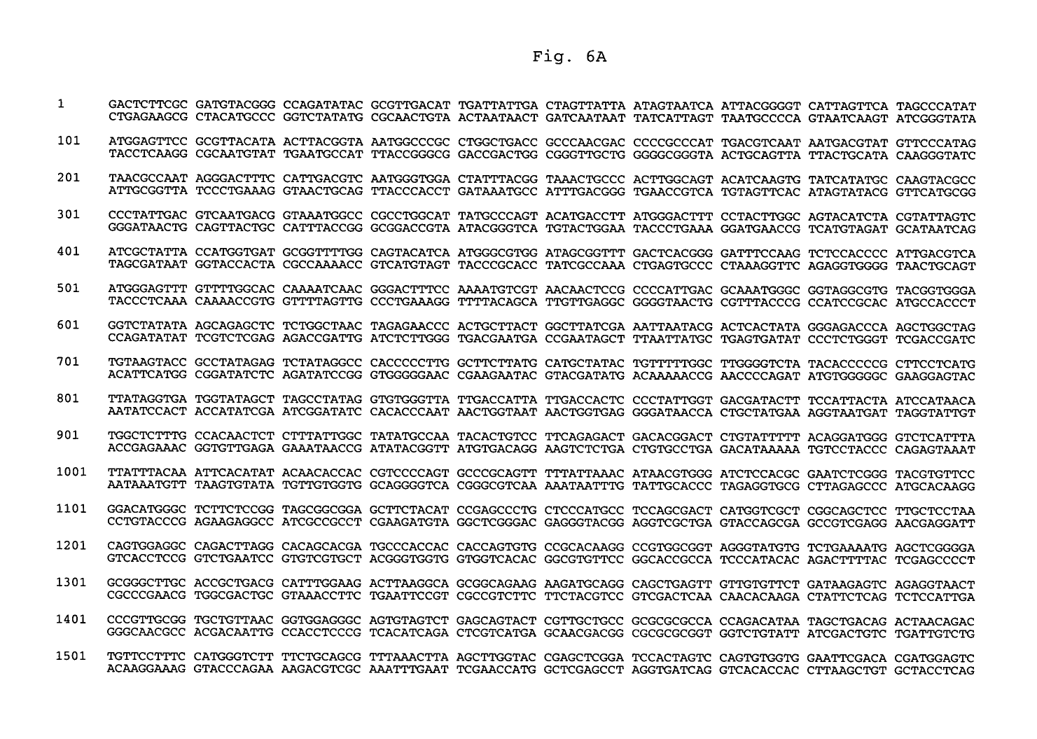

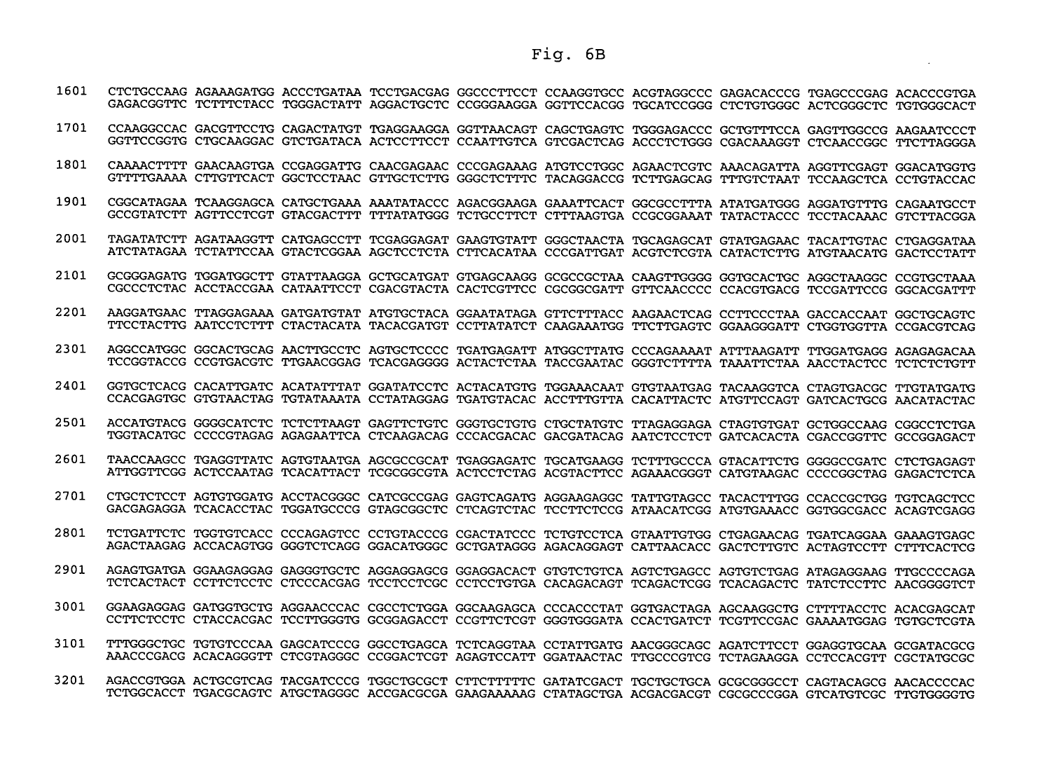

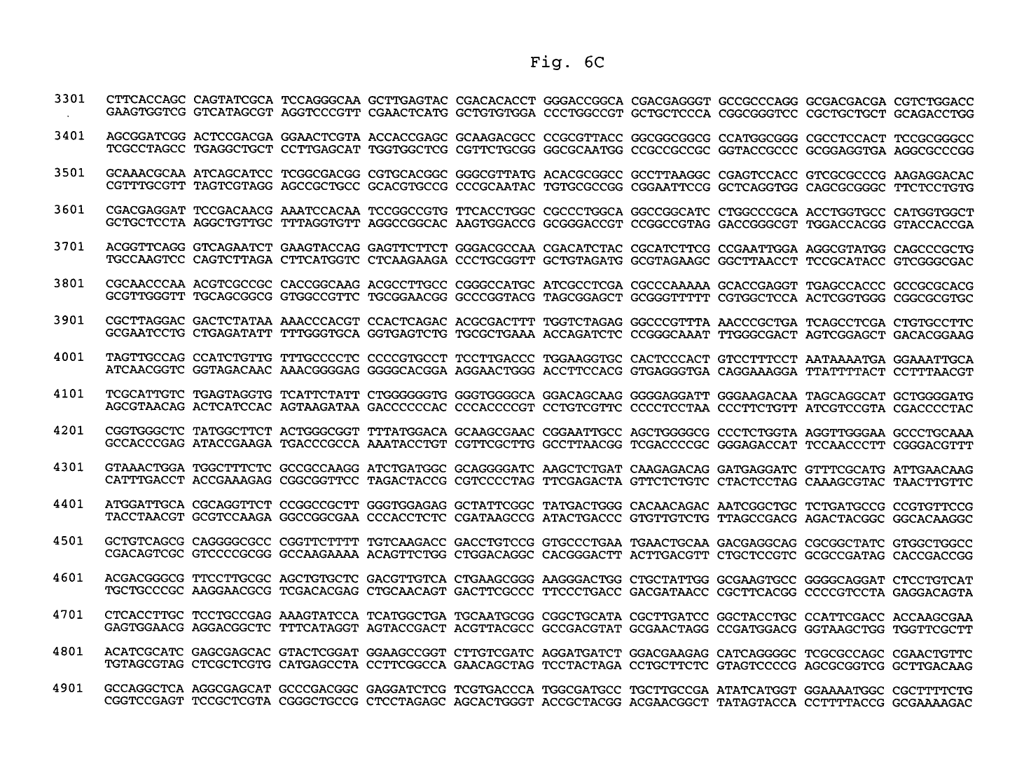

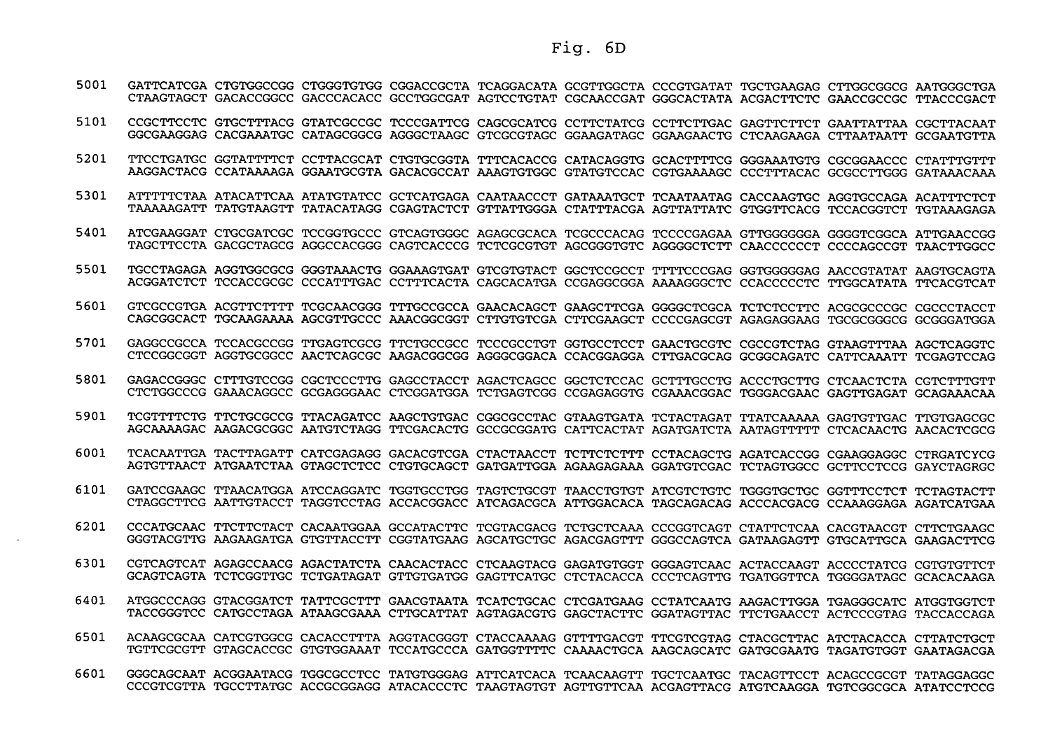

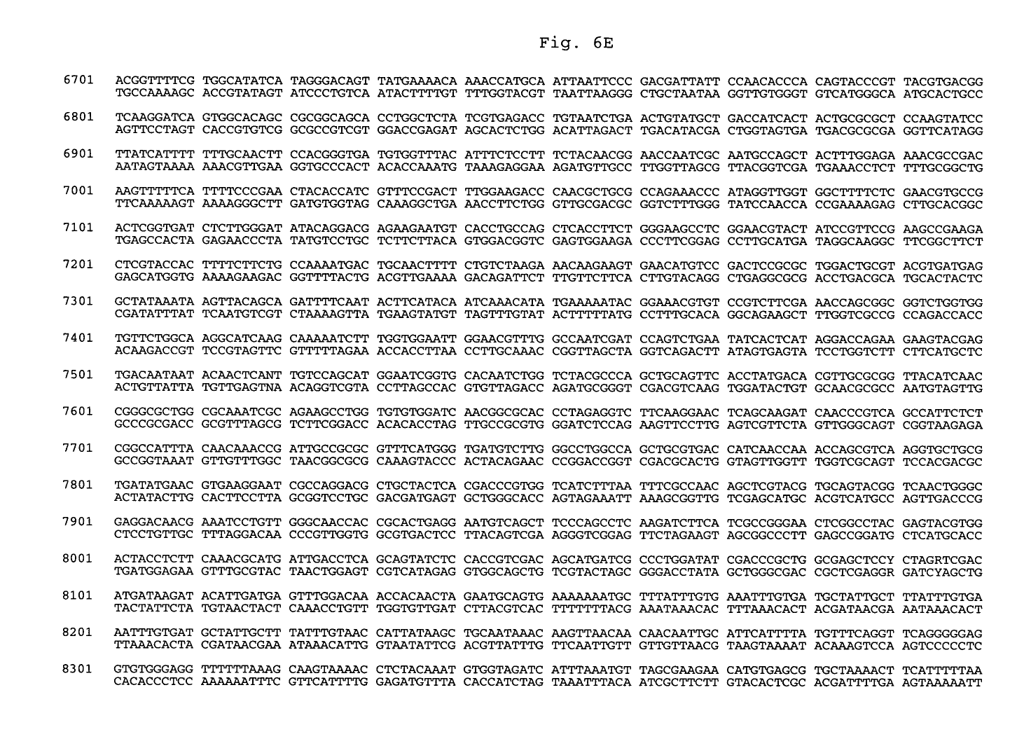

4. CMV-IEpp65mII-gB, which is a combined fusion of IE1 and pp 65mII plus a truncated gB which contains the immunologic domains. See FIG. 6 (SEQ ID NO:6).

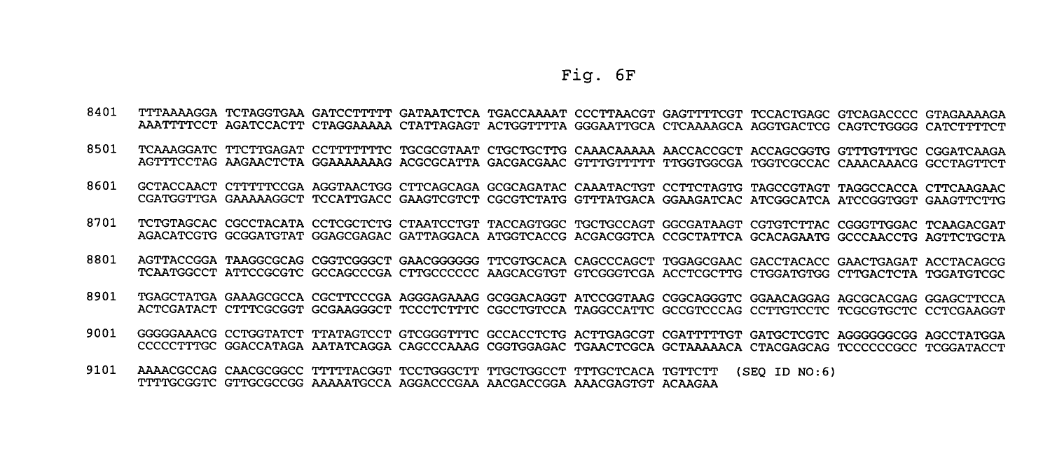

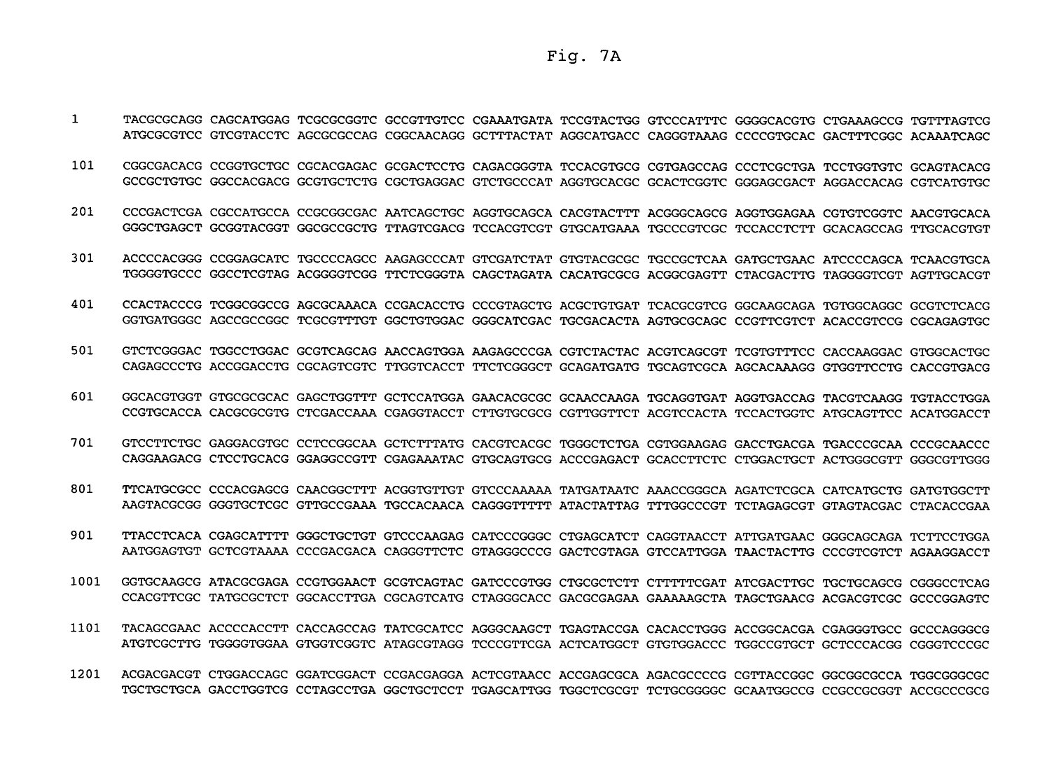

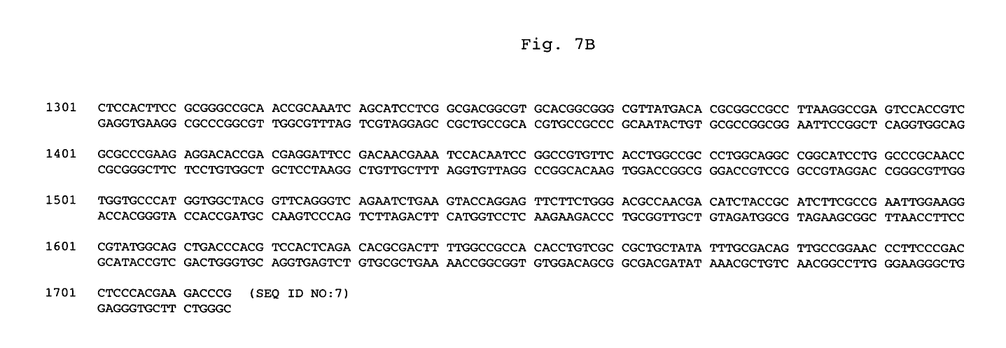

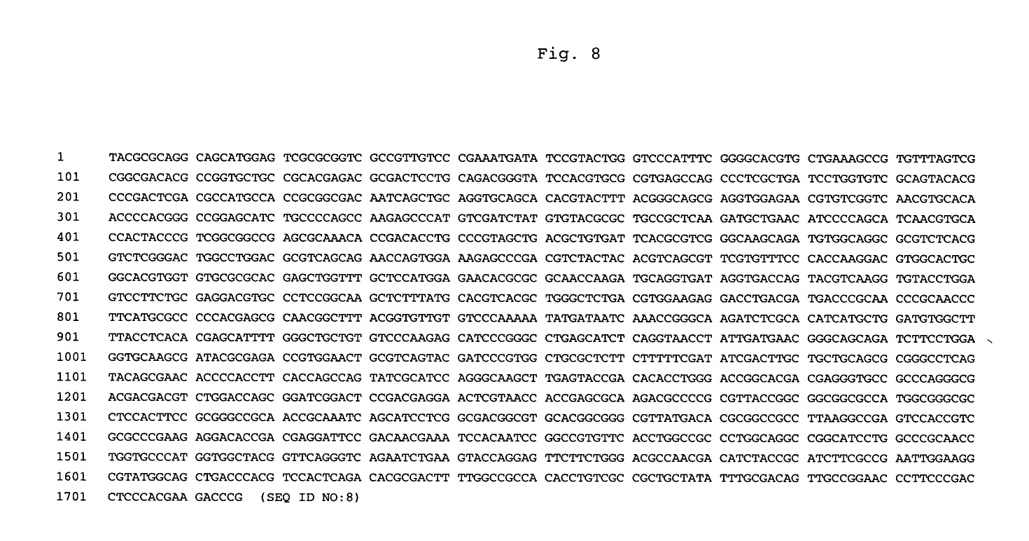

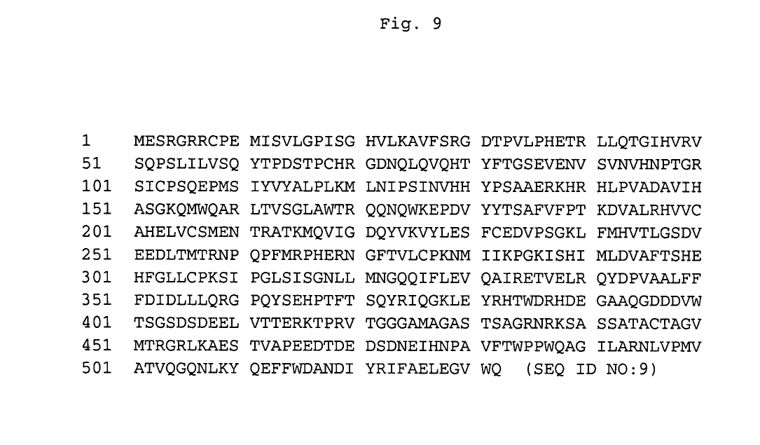

5. CMV-pp 65mII-NLS-KO (̂pp 65mII), which is a full length CMV pp 65mII with NLS-KO. See FIG. 7 (double-stranded; SEQ ID NO:7) and FIG. 8 (single-stranded; SEQ ID NO:8). FIG. 9 shows the protein sequence (SEQ ID NO:9).

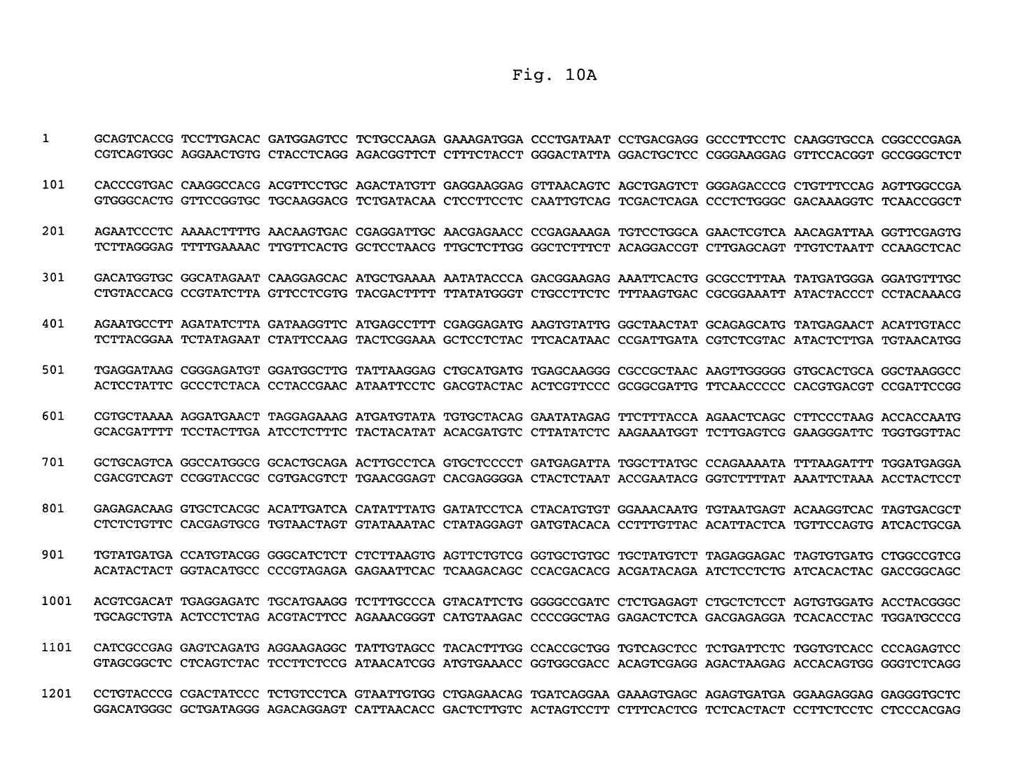

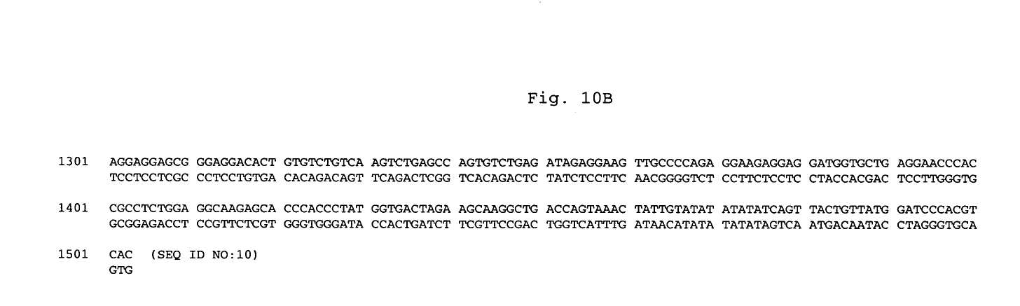

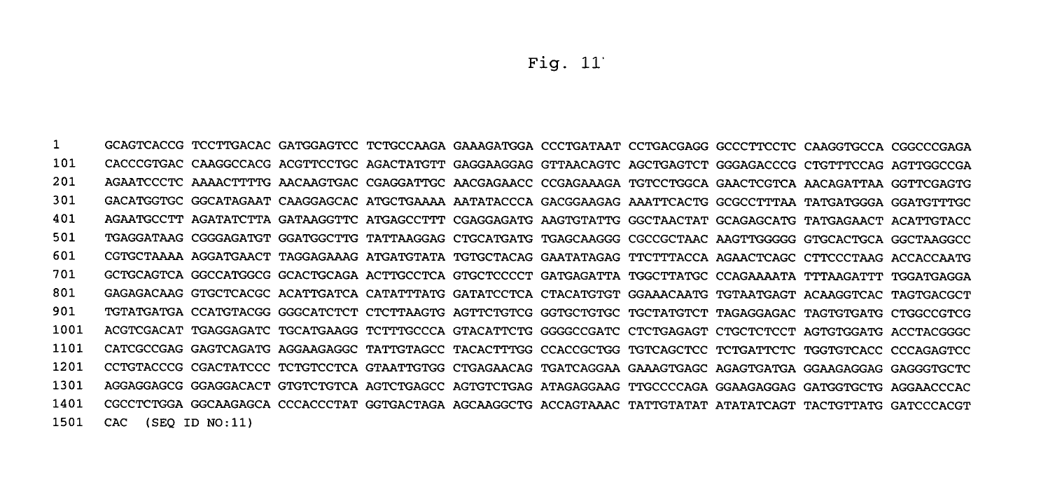

6. CMV-IE-NLS-KO (̂IE), which is a full length CMV IE1 with NLS-KO. See FIG. 10 (double-stranded; SEQ ID NO:10) and FIG. 11 (single-stranded; SEQ ID NO:11). FIG. 12 provides the protein sequence (SEQ ID NO:12).

7. CMV-IEpp65mII-NLS-KO (̂IÊpp 65mII), which is a fusion of IE1 and the truncated pp 65mII with NLS-KO in each. See simultaneously. Tetramer reagent binding to both genes was also observed by FACS. In summary, the vaccine vectors that contain an NLS-KO elicit robust CTL responses with improved protein expression and antigen processing/presentation in the target cells, without losing immunogenicity in a vaccine model.

The methods described here, therefore, in some embodiments, provide ways to improve to recombinant vaccines and target cells generically, to improve their immunogenicity, using a nuclear localization signal knock-out (NLS-KO) of at least one encoded target protein or polypeptide. Methods of the invention can be applied to any recombinant vaccine that encodes proteins or polypeptides that localize to the nucleus. Proteins and polypeptides referred to as “derived from a cytomegalovirus protein sequence” herein are CMV peptide sequences that may be found by analyzing the sequences using a publicly available computer program, e.g. BIMAS or SYFPEITHY, to locate sequences likely to be presented on MHC class 1 HLA A2. Analogous methods can be used to locate other sequences from other proteins or other viruses.

### EXAMPLES

### Example 1

### Nuclear Localization Signal Knock-Out (NLS-KO)

CMV-IE1 cDNA was inserted in pVAX1 (Invitrogen). The nuclear localization signal (NLS) in IE1 was found using a PSORT program and is located on exon 4 at amino acids 326 to 342. Its peptide sequence is KRPLITKPEVISVMKRR (SEQ ID NO:17). This sequence was removed by reverse PCR of the pVAX-IE1 plasmid with the following primers:

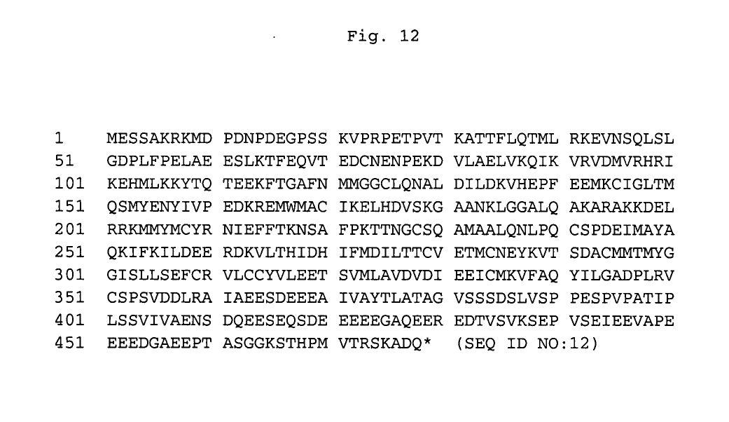

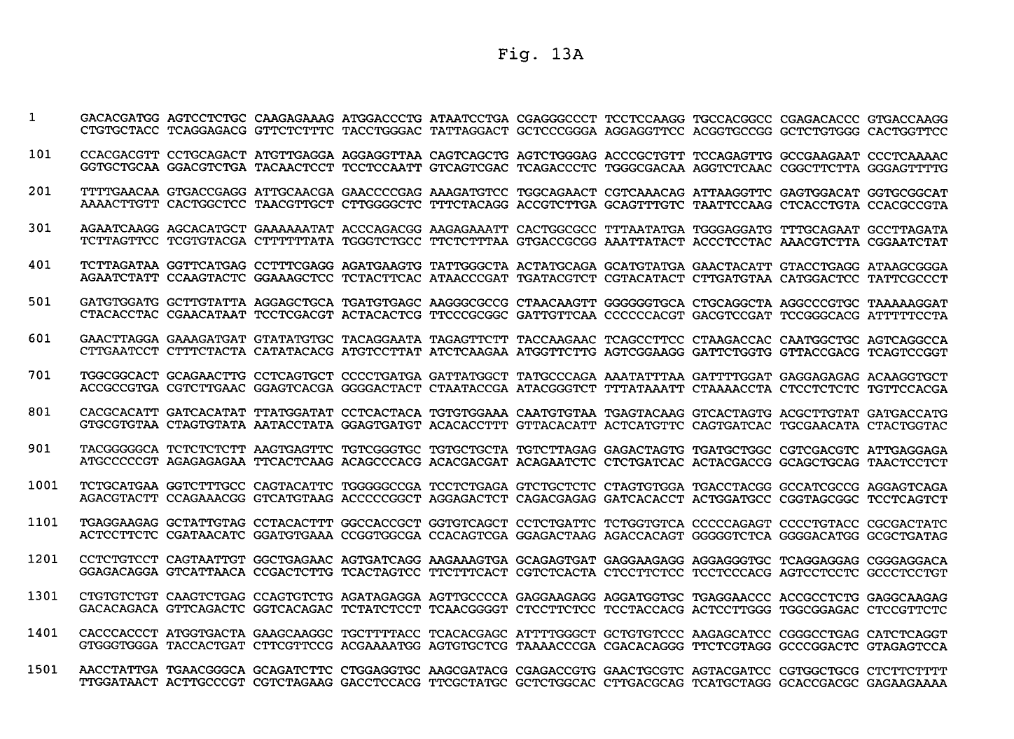

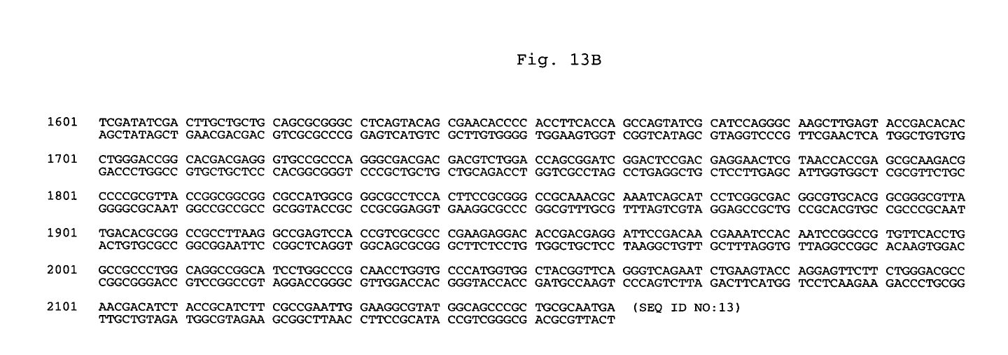

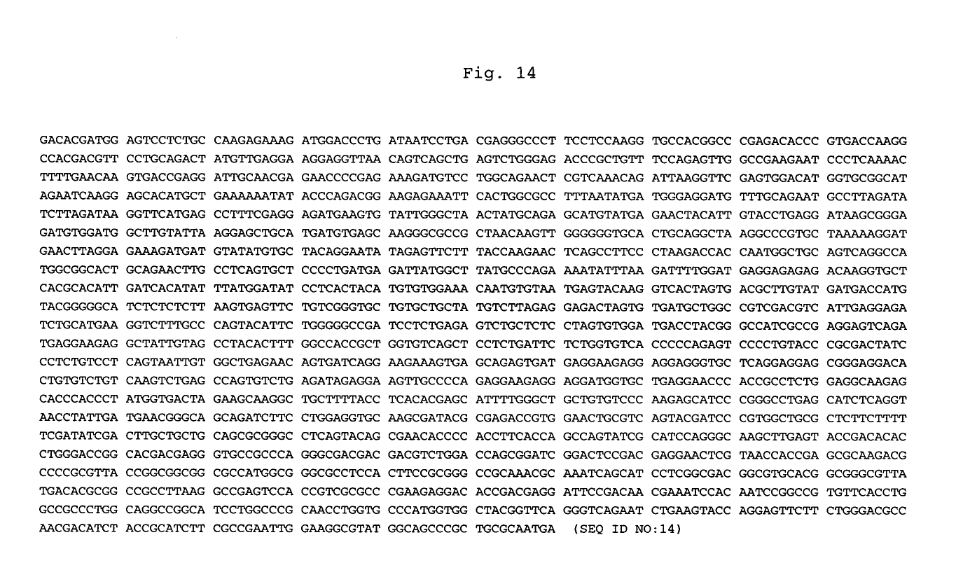

IE1-forward: 5′ GTCGACGGCCAGCATCACACTAGTCTCC (974-996; SEQ ID NO:21) containing a SalI site (underlined); FIG. 13 (double-stranded; SEQ ID NO:13) and FIG. 14 (single-stranded; SEQ ID NO:14). FIG. 15 shows the protein sequence (SEQ ID NO:15).

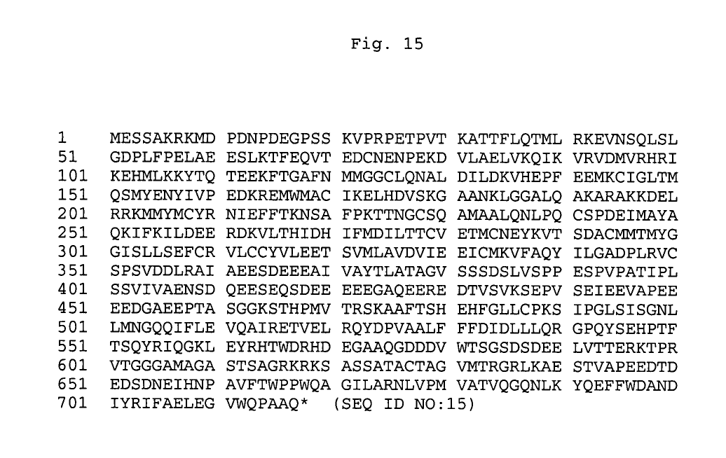

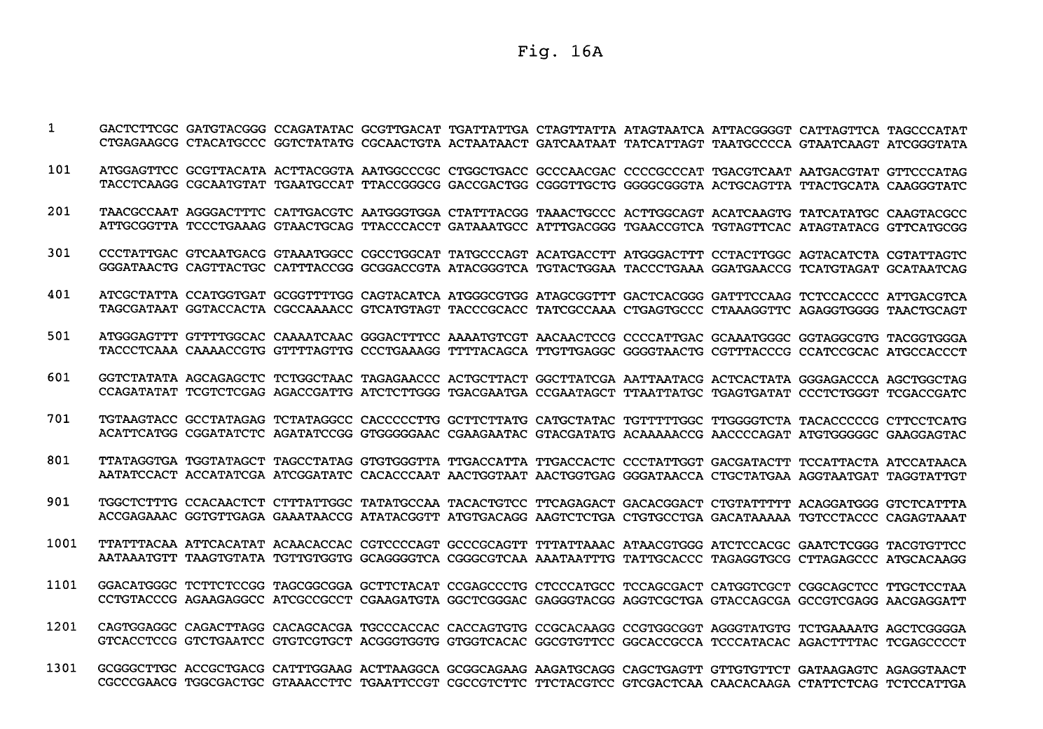

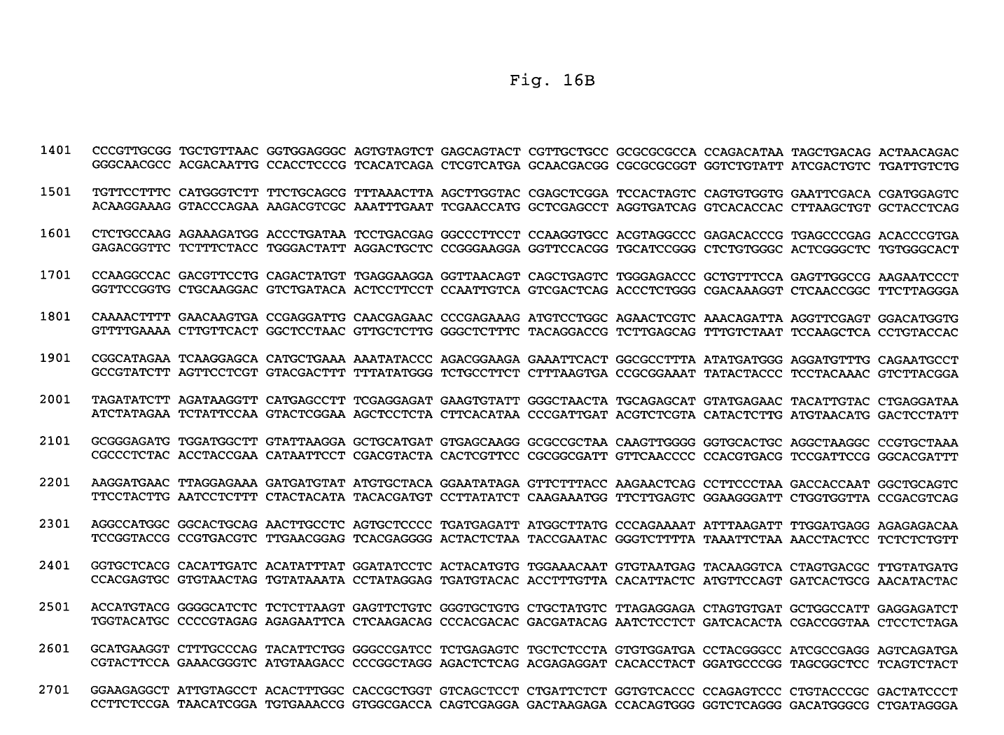

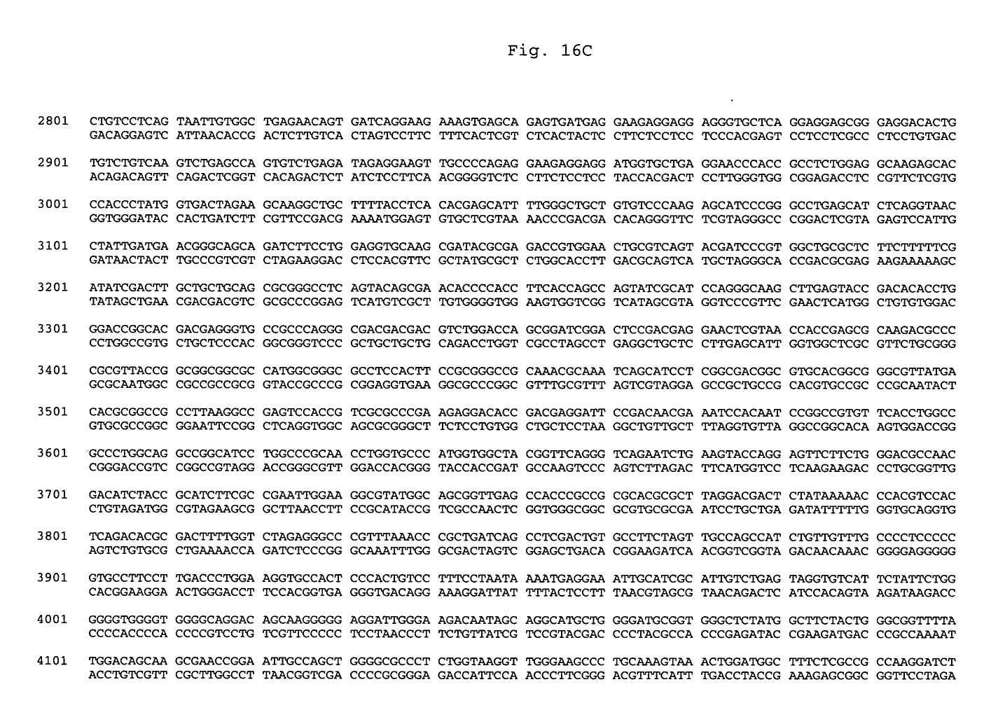

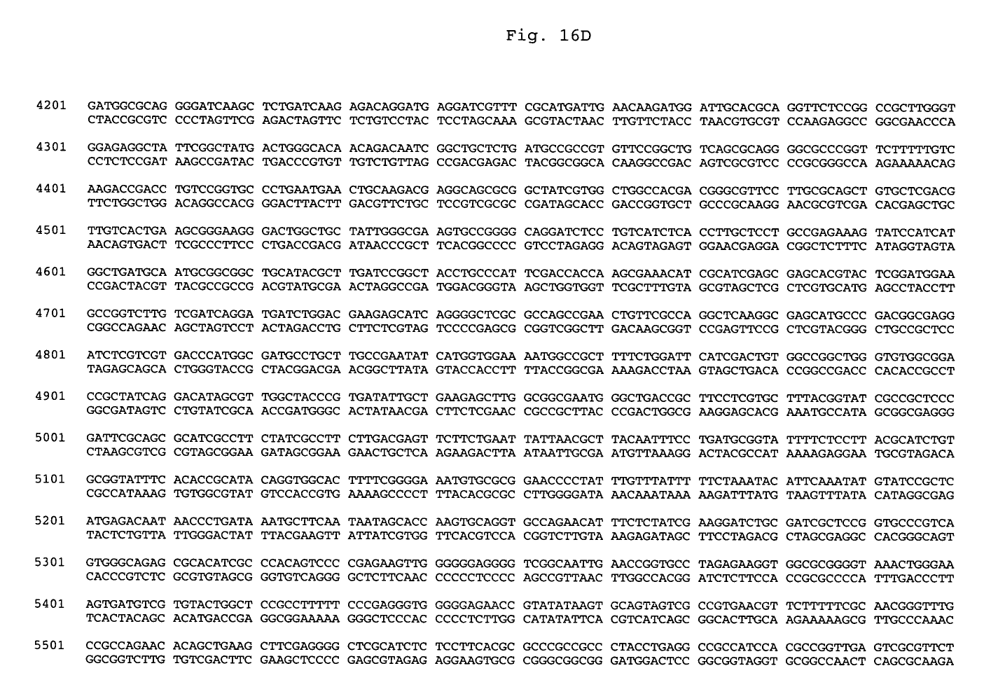

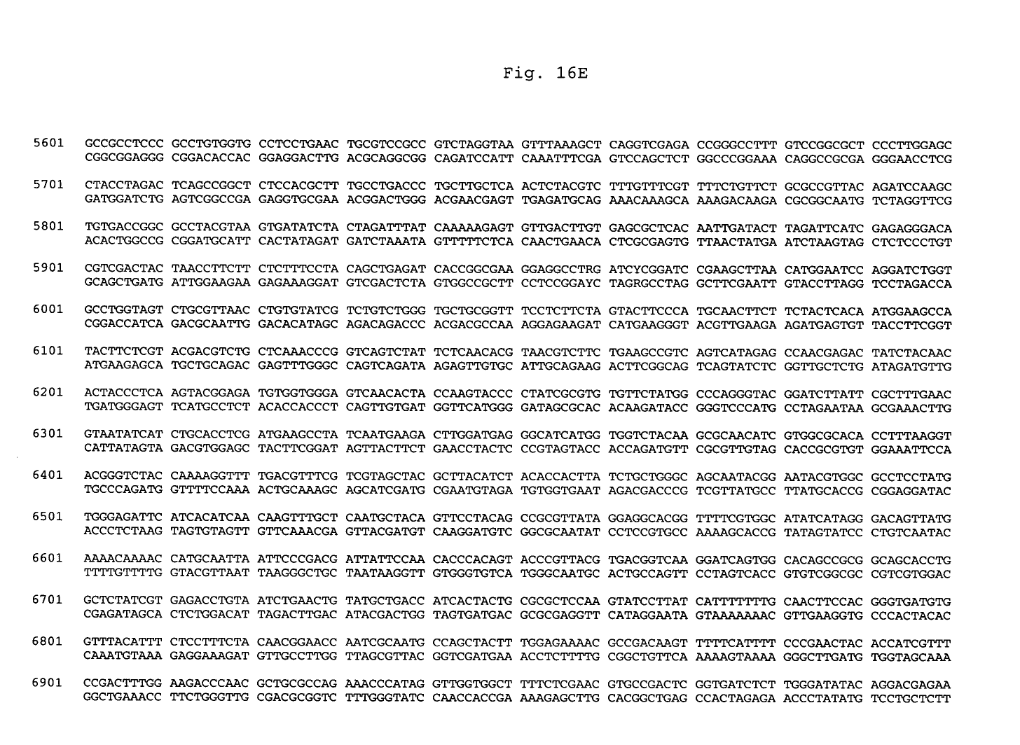

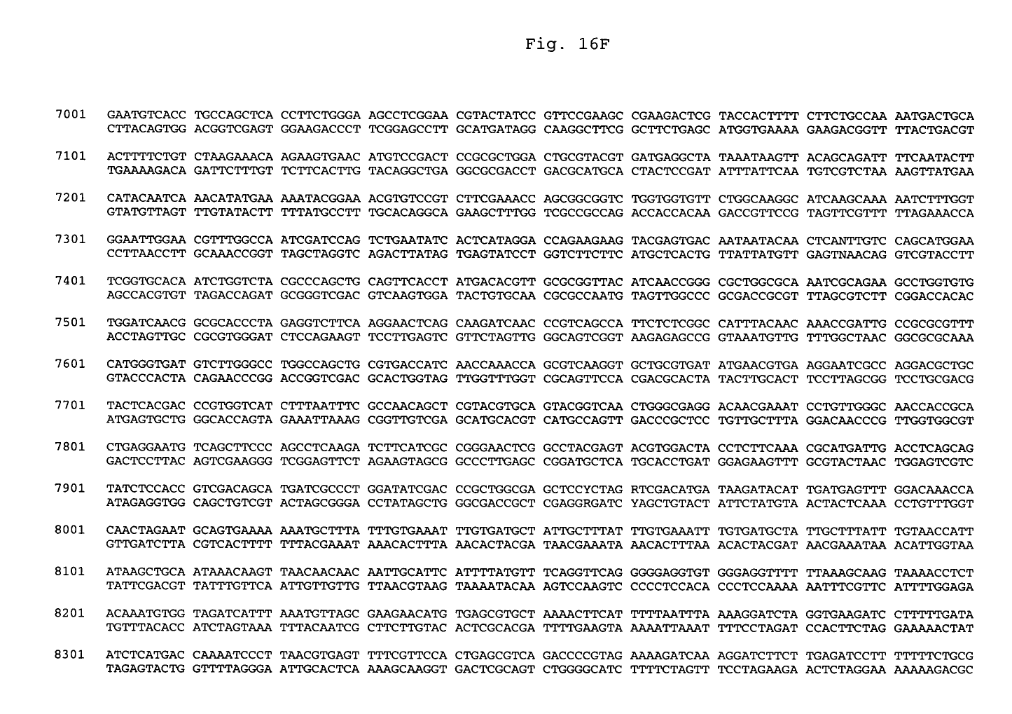

8. CMV-IEpp65mII-gB-NLS-KO (̂IÊpp 65mIIgB), which is a combined fusion of IE1 and pp 65mII with NLS-KO in each plus a truncated gB which contains the immunologic domains (SEQ ID NO:16). See FIG. 16. Adjacent regions also may be removed as well. An adjacent region is defined as the amino acids (or nucleotides) abutting the NLS sequence, for example 1-4 amino acids or codons, or more, such as 5-10 amino acid codons, for example.

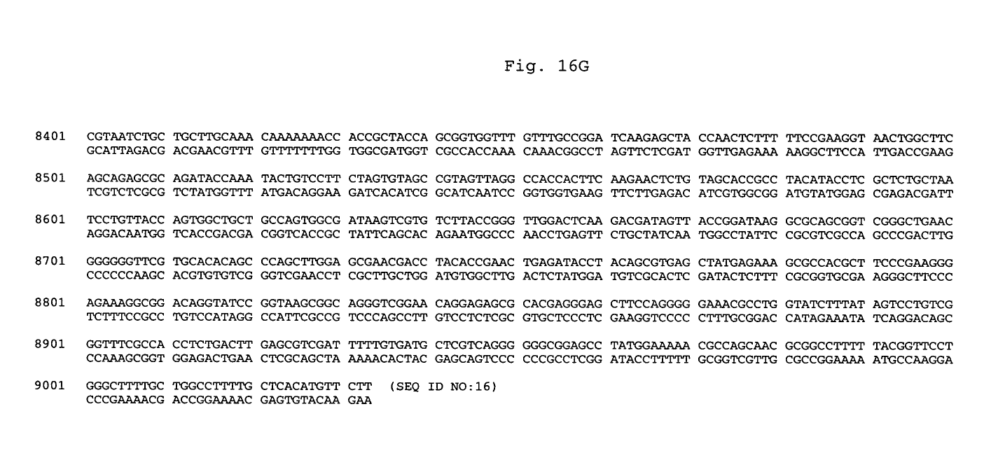

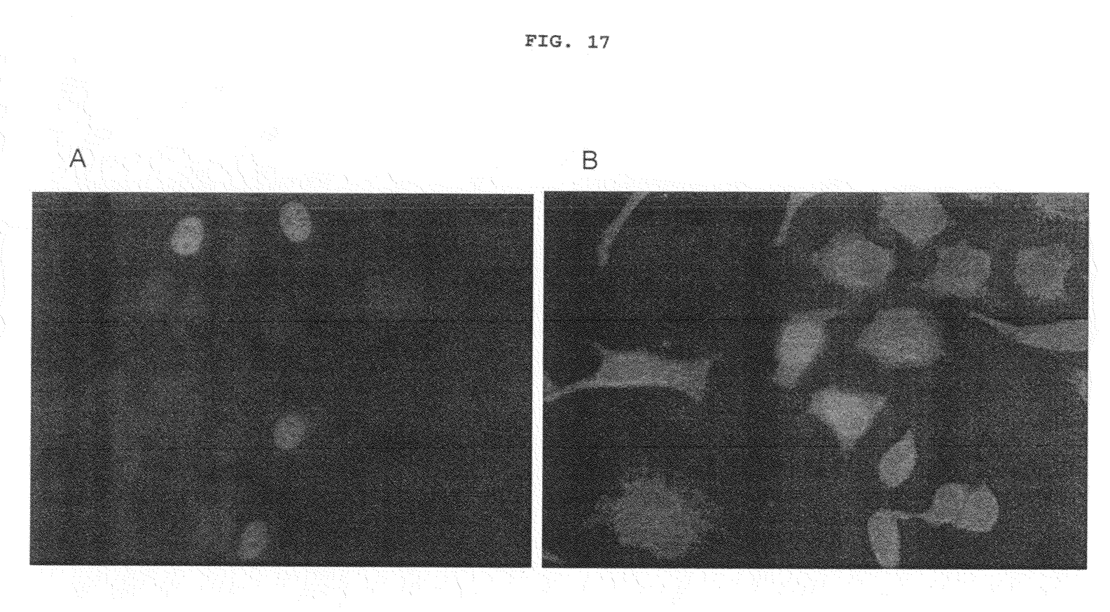

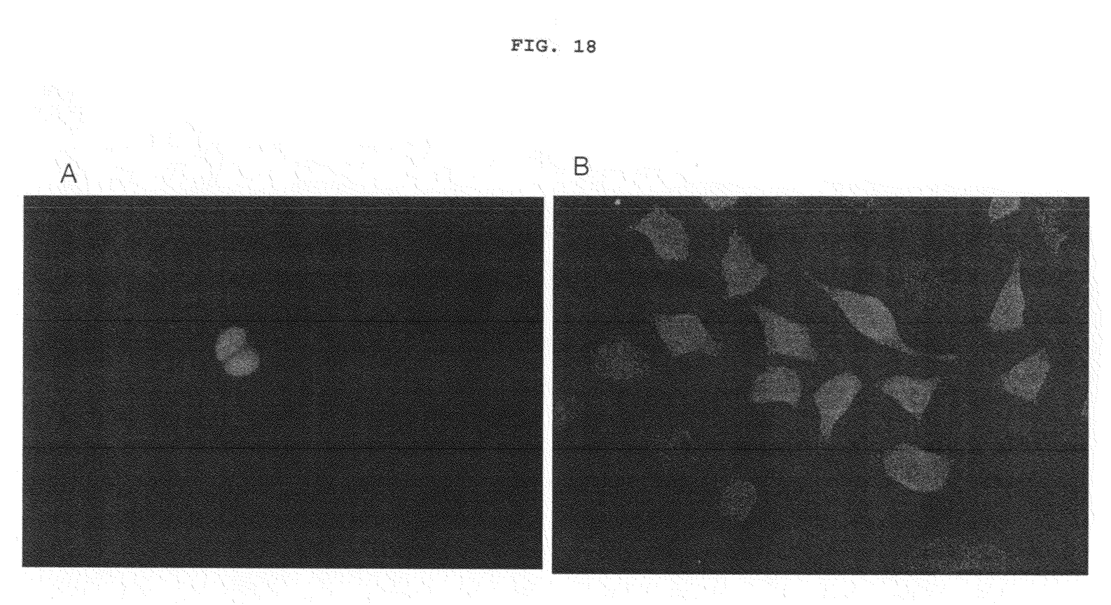

The NLS-KO constructs were made by reverse PCR with designed primers that amplified the whole plasmid but removed the NLS for each gene. See Example 1. The cytoplasmic localization of the NLS signal knock-out (NLS-KO) constructs was confirmed using a dual DAPI/IE1 or DAPI/pp 65mII stain by fluorescent microscopy. For this confirmation, HeLa cells were transfected with pVAXintIE (native) or pVAXintAIE (NLS-KO) and stained with anti-IE1 monoclonal antibodies, or transfected with pVAXintpp65mII (native) or pVAXintApp65mII (NLS-KO) and stained with anti-pp 65mII monoclonal antibodies. Results are shown in FIG. 17 (IE) and FIG. 18 (pp 65mII). Panel A of each Figure shows cells expressing the native sequence; panel B of each Figure shows cells expressing the NLS knock out. These constructs were inserted into rAAV.

To compare the immune responses elicited by the NLS-KO and intact constructs, HHDII mice (expressing the human HLA A*0201) were inoculated with the fusion DNA IE1pp 65 followed 4 weeks later by rAAV-IE1-pp 65. CTLs also were generated using either the NLS-KO constructs or the intact constructs, confirmed by chromium release assay (CRA) after stimulation with peptide-loaded blasts. Up to 90% target lysis was reached with cells from mice immunized with either vector, and the response was targeted to both CMV genes (IE1 and pp 65) IE1-reverse: 5, GTCGACATTGAGGAGATCTGCATGAAGG (1048-1069; SEQ ID NO:22) with a SalI site (underlined). The amplification product was cut with Sal1, gel purified, annealed, and then inserted into competent DH5a cells after the plasmid DNA was verified by DNA sequencing.

Similarly, the pp 65mII DNA, as modified according to Yao et al., Vaccine 19:1628-1635, 2001, the disclosures of which are hereby incorporated by reference, to remove the protein kinase activity was analyzed for localization of NLS motifs. See also U.S. Pat. No. 6,835,383 B2, which is hereby incorporated by reference for methods. This was done according to the NLS motif patterns previously described by Schmolke et al., J. Virol. 69:1071-1078, 1995, the disclosures of which are hereby incorporated by reference. Two NLS motif pattern sequences were found in CMVpp65mII. The first motif (at amino acids 415-438) contains the mutation K436N described by Yao et al. and therefore results in a kinase-deficient pp 65 protein. The motif sequence is RKTPRVTGGGAMAGASTSAGRK/NRK (SEQ ID NO:23; mutation underlined). This sequence was not modified further since it was previously modified as described by Yao et al. The second motif (located at amino acids 536-561; PKRRRHRQDALPPGPCIASTPKKHRG, SEQ ID NO:18) was removed using the following primers: pp 65-forward: 5′ TTGCGCAGCGGGCTGCCATACG (1601-1622, SEQ ID NO:24) and pp 65-reverse: 5′ TGACCCACGTCCACTCAGACACGCGAC (1741-1764; SEQ ID NO:25). The amplification product was confirmed by DNA sequence and handled as described with the IE1 plasmid.

### Example 2

### Construction of an IE1-pp 65 Fusion Protein

A DNA expression vector was constructed to express the immunogenic domain of CMV proteins, the main target for antibody and for CTL epitope recognition. The pVAX1 expression plasmid was used as the backbone. The target protein sequences were inserted at the ECORI and XbaI/XhoI sites as a CMV-IE1 and CMV-pp 65 fusion construct (IEpp65) made by PCR as follows.

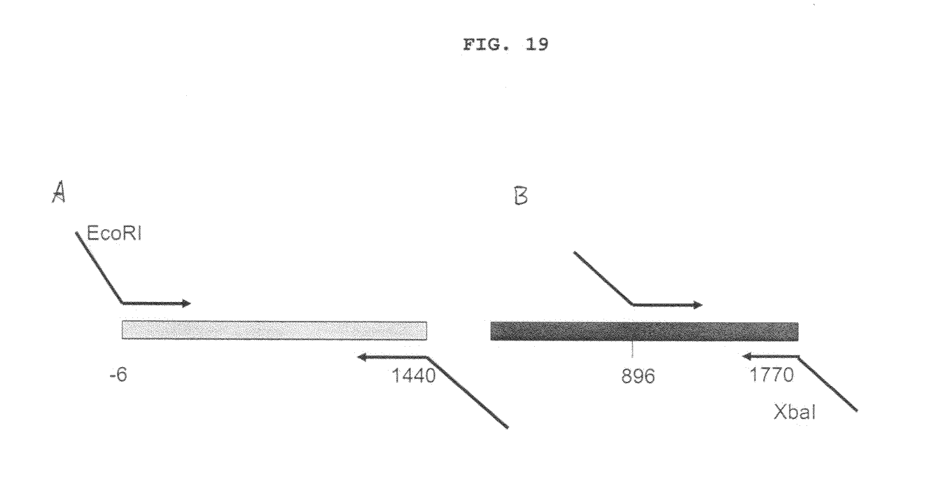

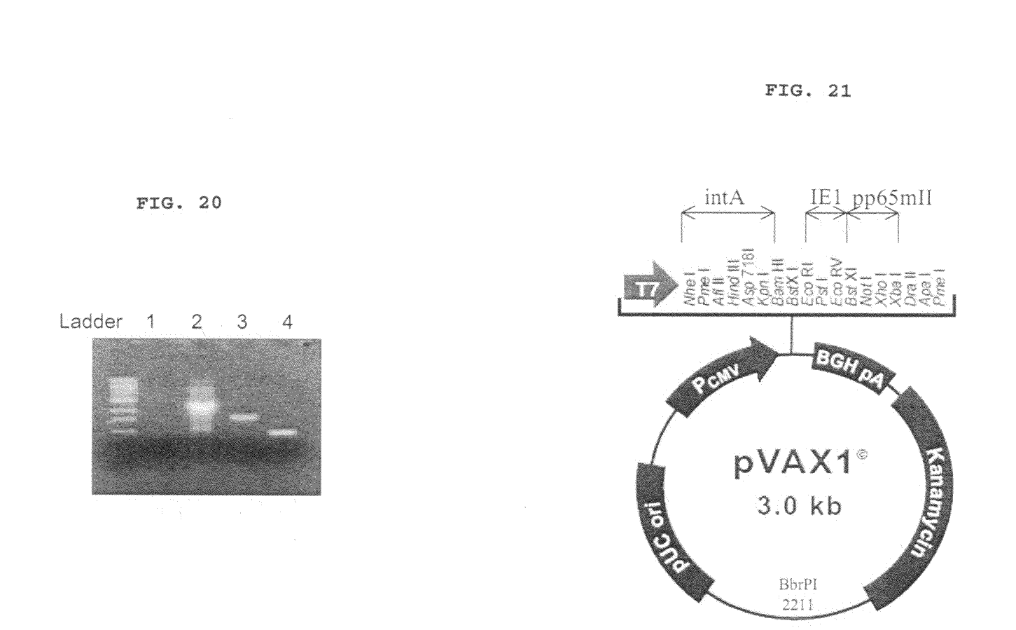

The CMV-IE1 gene was amplified with the forward primer (position −6 to +16; 5′ tacGAATTCgacacgatggagtcctctgcc 3′; SEQ ID NO:26), which contains the EcoRI site followed by the CMV-IE1 start site, and the reverse primer (base pair position 896-911; 5′ gtgtgaggtaaaagcagccttgcttctag 3′; SEQ ID NO:27) which contains the carboxy terminal of the IE1 gene product just in front of the stop site (1427-1440). See FIG. 19A. The CMV-IE1 gene then was linked in frame to CMV-pp 65 as an overhang starting at the corresponding amino acid 300. The CMV-pp 65 mutant II gene, described above and in Yao et al., was amplified using, as forward primer, the complementary sequence of the reverse primer IE1, with IE1 overhang (5′ ctagaagcaaggctgcttttacctcacac 3′; SEQ ID NO:28) and as reverse primer, containing the XbaI site, (5′ acttctagaccaaaagtcgcgtgtctgagt 3′; SEQ ID NO:29). See FIG. 19B. Each gene was amplified using a proof-reading Taq polymerase (such as Pfu Turbo (Stratagene)), gel purified (see FIG. 20), and amplified using the IE1-forward primer and pp 65-reverse primer for 33 cycles. The final product was cut with the appropriate enzymes, EcoRI and XbaI, and was inserted into the expression vector pVAX1. See FIG. 21.

Preliminary expression results showed that both CMV-IE1 and CMV-pp 65 genes were expressed similarly in transfected HEK-293 cells using protein specific monoclonal antibody. Using this fusion product allowed the IE1-pp 65 insert to be shortened by 1 kb. Therefore this is an appropriate cornerstone for subsequent DNA and rAAV constructions. The NLS-KO fusion construct was made in an analogous manner with the same primer sequences.

### Example 3

### Recombinant Adeno-Associated Virus Constructs

The assembly of the rAAV has been described previously using a rAAV2 vector CWCMV. See Gallez-Hawkins et al., Vaccine 23:819-826, 2004, the disclosures of which are hereby incorporated by reference. DNA constructs were inserted into the multiple cloning site (MCS) of rAAV. To further purify the rAAV from cellular proteins, the viral stock was adjusted to pH 8-8.5 and 1 mM MgCl₂ was added to facilitate DNAse treatment. Benzonase was added at a concentration of 225 U/2×10⁷ cells and incubated at 37° C. for 1 hour. This step removed the cellular chromosomal DNA. Trypsin was added at a final concentration of 0.25% and incubated at 37° C. for 1 hour to remove the cellular proteins, and the trypsin was inactivated with 1:10 volume fetal bovine serum (FBS).

The following steps were used to obtain a purified rAAV using a CsCl₂ gradient. CsCl₂ (0.522 g/mL) was added to the lysate in tubes to obtain a density of 1.42 g/mL. The tubes were subjected to 35,000 rpm for 72 hours in an SW41 rotor. One milliliter fractions having a refractory index (R.I.) of 1.37 were collected and pooled, then desalted using an Ultracel™ centrifugal filter device (Millipore™, Carrightwohill, Ireland). The rAAV then was titered on HT1080 cells. Generally, the process, from generating the virus by transfection of HEK293 cells to the last purification step, takes about 2 weeks.

An alternative method of AAV purification developed by Virapur™, which uses an ion exchange filtration cartridge to purify the rAAV2 in one afternoon also may be used. This method of purification yields more virus of similar infectivity. The Viripur ion exchange column method of purification is the preferred method for rAAV purification but either is sufficient for recovery of infectious rAAV.

### Example 4

### Improved Expression of Recombinant Protein

When vaccines based on CMV-pp 65mII, CMV-IEpp65mII, CMV-pp 65mII-NLS-KO or CMV-IEpp65mII-NLS-KO were expressed from rAAV vectors, the expression of pp 65-specific protein was significantly increased. HT1080 cells were transduced with rAAV expressing the pp 65mII, pp 65mII-NLS-KO, IEpp65mII and IEpp65mII-NLS-KO proteins. Expression of pp 65 was then measured qualitatively by electrophoresis of the cell extracts with staining using anti-pp 65 monoclonal antibodies. The results are shown in FIG. 22. Bands specifically stained are marked with an asterisk.

### Example 5

### Increased Killing of Transduced Target Cells by Cytotoxic T Lymphocytes

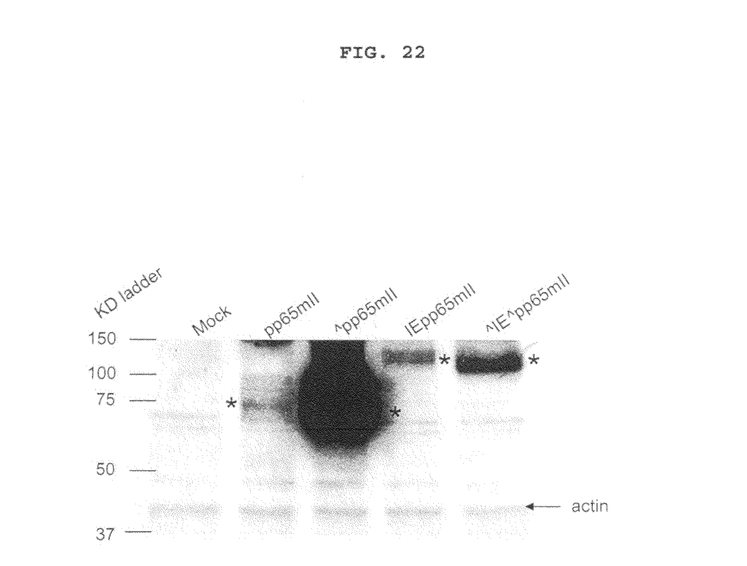

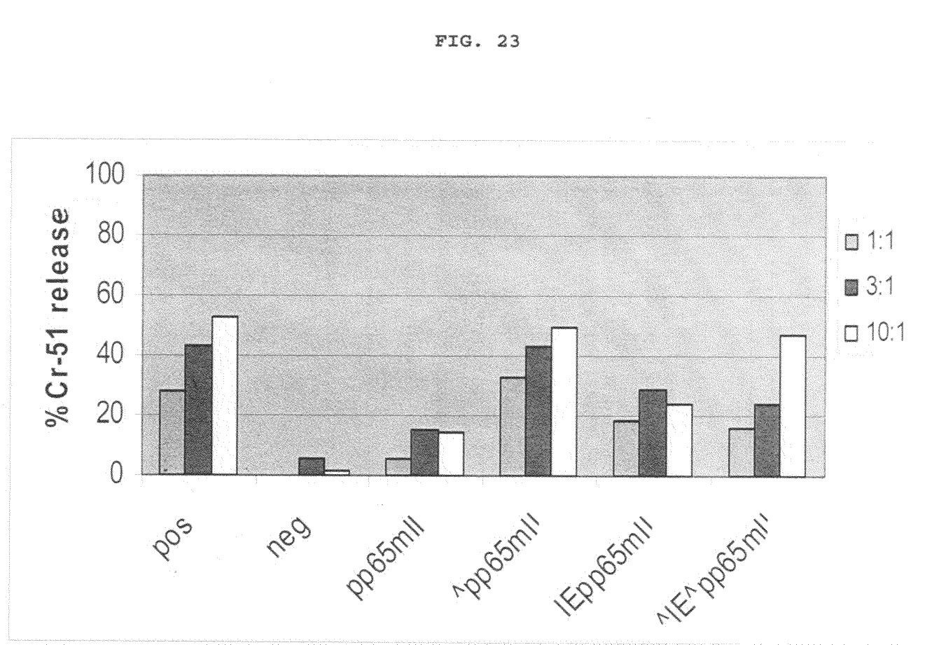

HLA A*0201 target cells (A293 cells) were produced as follows. The cells were transduced with rAAV expressing native or NLS-knock out proteins to express the following proteins: pp 65mII, pp 65mII-NLS-KO, IE(N) pp 65mII and IE-NLS-KOpp65mII-NLS-KO. After a 48-hour incubation with the rAAV expressing the CMV genes, the target cells were loaded with ⁵¹Cr for 1 hour and then cultured at the indicated effector:target ratios (1:1, 3:1, and 10:1) for 4 hours with human CTL specific for pp 65 peptide (NLVPMVATV; SEQ ID NO:30) recognition in the HLA A*0201 context. The ⁵¹Cr release in the supernatant, compared to control, indicates the amount of target killing performed by the CTL. More killing means that more pp 65 peptide was presented on the transduced targets with a A*0201-restricted CTL clone specific for HLA A2-p 495 (a peptide of HCMV pp 65 that is presented on the HLA A*0201 molecule; NLVPMVATV; SEQ ID NO:30. Results are provided in FIG. 23, and show that knock out of the NLS increased killing of the target cells by CTL.

### Example 6

### Murine Immunizations

The HHD II mouse expresses a transgenic monochain HLA class I molecule in which the C terminus of the human β2 m is covalently linked to the N terminus of a chimeric heavy chain (HLA-A-0201 α1-α2, H-2 D^{b} α3-transmembrane and intracytoplasmic domains). The H-2D^{b} and mouse β2m genes have been disrupted by the homologous recombination. See Pascolo S., Expert Opin. Biol. Ther. 5:919-938, 2005; Pascolo et al., J. Exp. Med. 185:2043-2051, 1997, the disclosures of both of which are hereby incorporated by reference. This mouse model was used to compare relative vaccine potency on an HLA A2 background.

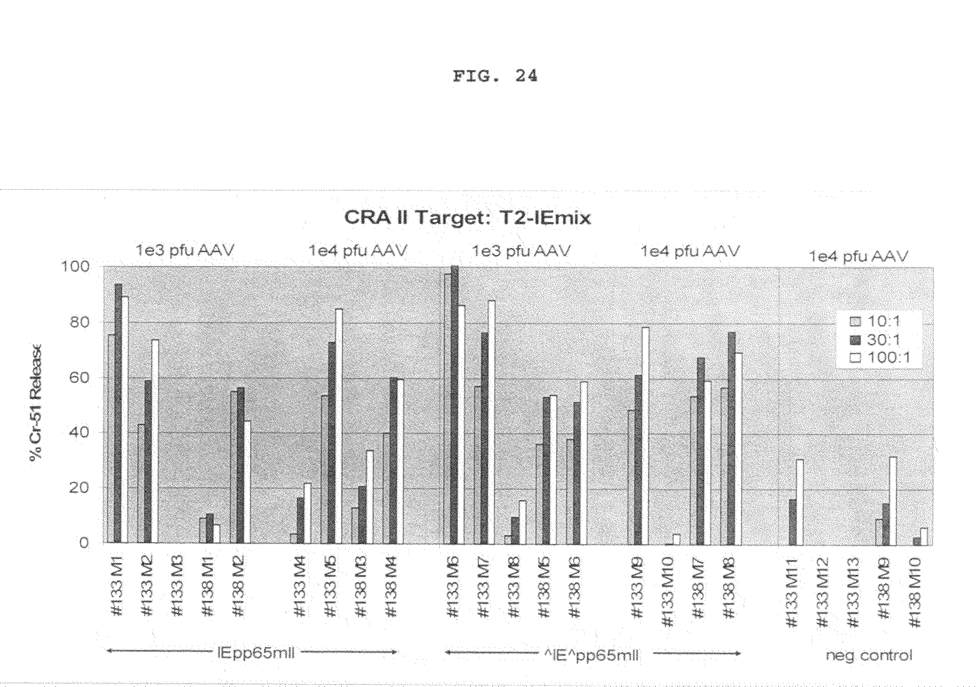

The murine immunizations are performed essentially as described in Yao et al., Vaccine 19:1628-1635, 2001; Gallez-Hawkins et al., Vaccine 23:819-826, 2004; and Gallez-Hawkins et al., Scand. J. Immunol. 55:592-598, 2002, the disclosures of all of which are hereby incorporated by reference. The 6-to-8-week-old mice (n=4 per variable) were injected I.M. with 50 μg of endotoxin-free DNA (prepared according to the Qiagen™ protocol), diluted in a total volume of 100 μl sterile saline, 50 μl in each thigh. Using the prime-boost model, 50 μg of pVAX1-CMV DNA was co-injected with pVAXintAgm-CSF DNA (50 ug pVAXIE1pp 65 and 50 ug pVAXgm-CSF per mouse) without gm-CSF adjuvant (prime), followed by injection with rAAV containing the same CMV-DNA at various titers ranging from 1×10² IU/mouse to 1×10⁵ IU/mouse (boost). Typically, the mice were injected intramuscularly with the indicated DNA on day 0 followed by the boost 4 weeks later, however this can be modified to optimize the schedules. The spleens were harvested 20 days after the boost and processed for chromium release assay (CRA). Sera were collected at this time for antibody detection by ELISA. Other organs were collected as needed, for example the draining lymph nodes (lumbar, inguinal and popliteal nodes). AS indicated in FIGS. 24 and 25, this regimen of vaccination was immunogenic and induced T cell responses specific for CMV. Vaccine regimens such as that described here with DNA priming and AAV boosting, could be used in humans to induce immunity or boost immunity to CMV.

### Example 7

### In Vivo Peptide/Target Cell Production and Chromium Release Assay (CRA)

Overlapping peptides used in the immunologic assays were purchased from Jerini™ (Germany) for CMV-pp 65 (pepmixpp65) and CMV-IE1 (pepmix IE1). These peptides were used to label autologous splenocytes to stimulate murine effector cells (see Example 6) in an in vitro stimulation (IVS) culture for 5-7 days. In the context of HLA A*0201, specific peptides (pp 65 495 (NLVPMVATV, SEQ ID NO:30) or IE1-297 (TMYGGISLL, SEQ ID NO:31), IE1-256 (ILDEERDKV, SEQ ID NO:32) or IE1-316 (VLEETSVML, SEQ ID NO:33)) were used to label ⁵¹Cr-loaded target cells (T2 cells, LCLA2 or EL4A2 cells). These targets were challenged subsequently with in vitro-stimulated effector cells and the specific lysis measured in a chromium release assay.

The protocol described here was performed generally as published. See Gallez-Hawkins et al., Scand. J. Immunol. 55:592-598, 2002 and Benohamed et al., Hum. Immunol. 61:764-779, 2000, the disclosures of which are hereby incorporated by reference. Briefly, three days before the harvest of effector cells from immunized HHD II mice, blasts cells were prepared from syngeneic spleen cells (1 spleen for 3 immunized mice), cultured at a concentration of 1×10⁶ cells/mL and stimulated with 25 pg/mL lipopolysaccharide (LPS) (Sigma, St Louis, Mo.) and 7 μg/mL of dextran sulfate (Sigma, St Louis, Mo.).

The procedure for in vitro stimulation (IVS) of spleen cells was as follows. LPS-stimulated blasts, resuspended at a concentration of 25×10⁶ cells/0.2 mL serum-free RPMI were incubated with 100 μM CMV-pp 65 or CMV-IE1 peptide and 3 μg/mL β2-microglobulin at 37° C. for 4 hours. The cells were irradiated at 3000 RADS using an Isomedix™ Model 19 Gammator (Nuclear Canada™, Parsippany, N.J.). The CMV-pp 65 or CMV-IE1 peptides, were 95% pure, established by HPLC. The peptide-loaded blast cells then were plated in a 24-well plate (1×10⁶ blasts and 3×10⁶ spleen cells from immunized mice per well) in complete RPMI supplemented with 10% rat T-stim™ culture supplement (Becton-Dickinson™, Franklin Lakes, N.J.). A second IVS was performed 7 days later using the same protocol.

The CRA was performed using target cells that presented the HLA-A*0201 allele (T2 cells (ATCC CRL-1992), LCL-A2 (an EBV transformed human B cell line), EL4A2 (a mouse H-2b cell line stably transfected with the A2 gene) and LCL-A3, (a control HLA cell line)). The cells were labeled with A2 specific peptides or infected either with CMV-Towne overnight at MOI=5 in 200 μl of medium for 3 hours at 37° C. or transduced for 48 hours with rAAV expressing various constructs (for human fibroblasts) or infected with rVacpp65 at MOI=5 (for autologous mouse cells). ⁵¹Cr (200 μCi) then was added. The labeled target cells were washed and then mixed at Effector:Target (E/T) ratios ranging from 100 to 10 in a 96-well U-bottom plate with effector cells in triplicate wells, and incubated for 4 hours. Aliquots of the supernatant were counted using a Topcount™ counter (Packard Instrument™ Co, Downers Grove, Ill.). Representative results are provided in FIG. 24 and FIG. 25.

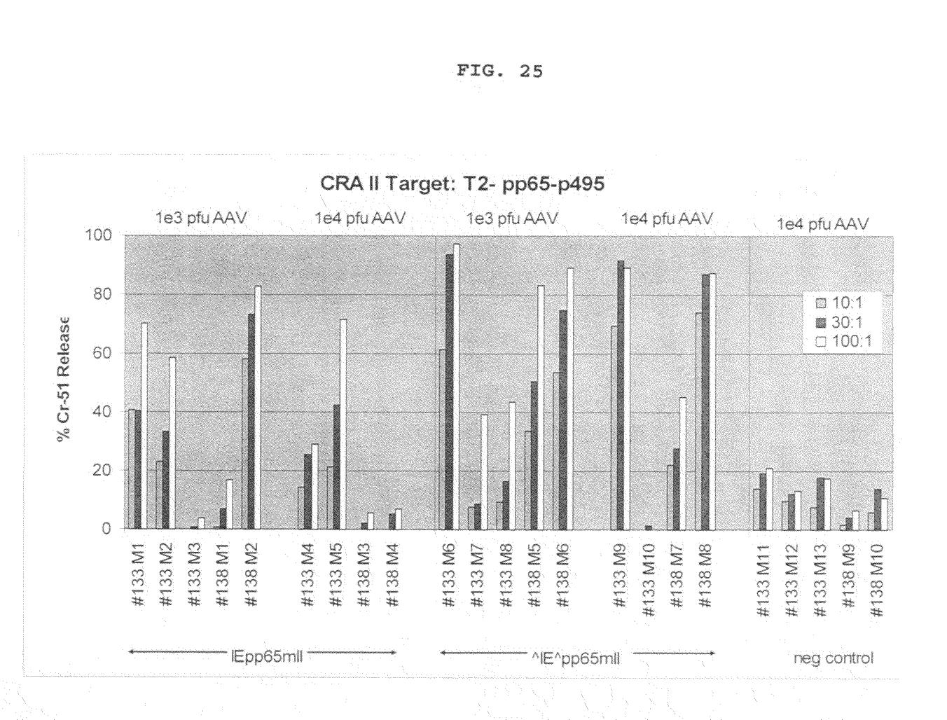

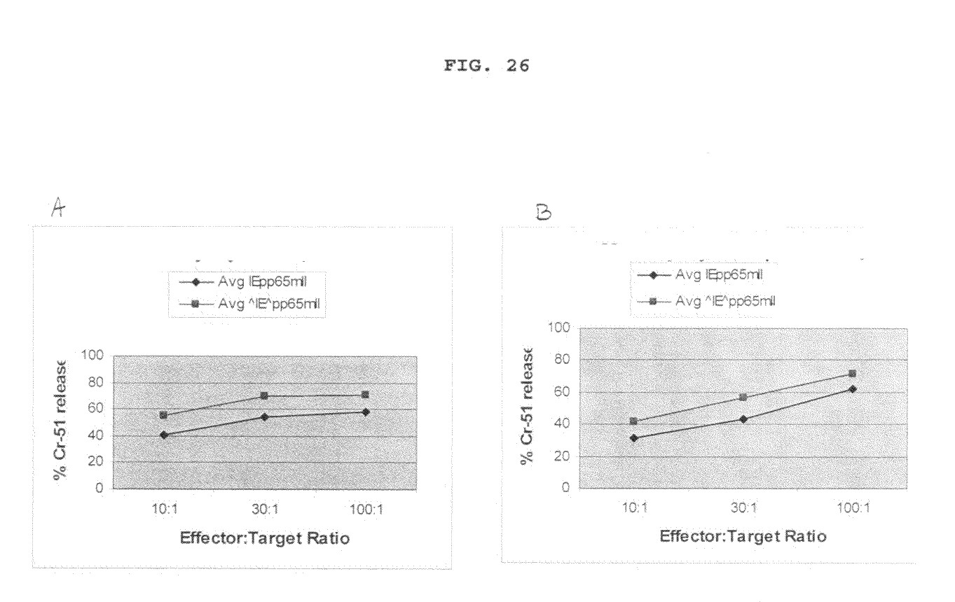

FIG. 24 shows killing (% ⁵¹Cr release) of target cells presenting IE Peptides by in vitro-stimulated murine splenocytes after vaccination with DNA expressing the IEpp65mII fusion protein (native sequence and NLS-KO). ̂ indicates NLS-KO in the gene which follows. FIG. 25 shows the same data for target cells presenting pp 65-p 495 (NLVPMVATV; SEQ ID NO:30). FIG. 26 is a summary of the data, showing the mean immune response to the CMV proteins. The results show that vaccination with the fusion constructs elicited CTL in vivo which were able to kill target cells expressing either CMV protein peptides and that NLS knock out improved this killing.

### Example 8

### Expansion of T Cells

To induce expansion of human T cells, human fibroblasts such as MRC-5 (HLA A*0201) cells are transduced for 24-48 hours with rAAV expressing the CMV gene products described herein. The cells are irradiated according to known methods and co-cultured with HLA A*0201 peripheral blood lymphocytes (PBL) for 5-10 days. The PBL are counted at the beginning and end of the culture to estimate the doubling time (proliferation). For HLA A*0201 or other HLA types, autologous dendritic cells can be transduced by the same methods to present antigen to PBL.

### Example 9

### HLA-Peptide Tetramer Reagent Binding Assay

Tetramer reagents for HLA A*0201 were prepared essentially as described in Lacey et al., Transplantation 74:722-732, 2002, the disclosures of which are hereby incorporated by reference. MHC tetramers are recombinant class I molecules, biotinylatd by bacterial BirA, folded with the peptide fo interest and β2M and then tetramerized using a fluorescent-labeled streptavidin which binds four biotins. These reagents, referred to simply as “tetramers,” can be used to quantitate numbers of antigen-specific T cells, especially CD8+T cells. Here, the reagents were folded using a CMV peptide specific for the pp 65 protein (pp 65-495: NLVPMVATV; SEQ ID NO:30) or for the CMV-IE1 protein (IE1-297: TMYGGISLL; SEQ ID NO:31) and conjugated with streptavidin-allophycocyanin (APC) (Molecular Probes™). These tetramer reagents specifically label T cells that express T cell receptors specific for a given peptide-MHC complex. For example, the NLVPMVATV tetramer binds to CMV virus-specific CTL from HHDII mice in these assays. Antigen-specific responses can be measured as CD8+, tetramer+T cells as a fraction of all CD8+lymphocytes.

For binding, one microgram of tetramer reagent was incubated for 1 hour on ice in the dark with 3×10⁵ splenocytes. After washing with PBS-0.5% BSA, the cells were labeled with FITC-conjugate murine CD8 antibody (Pharmingen T) for 20 minutes on ice in the dark, washed, resuspended in sheath fluid and analyzed with a FACScalibur™ flow cytometer (Becton Dickinson™, San Jose, Calif.). The lymphocyte gate was set based on forward and side scatter and a minimum of 50,000 events were captured.

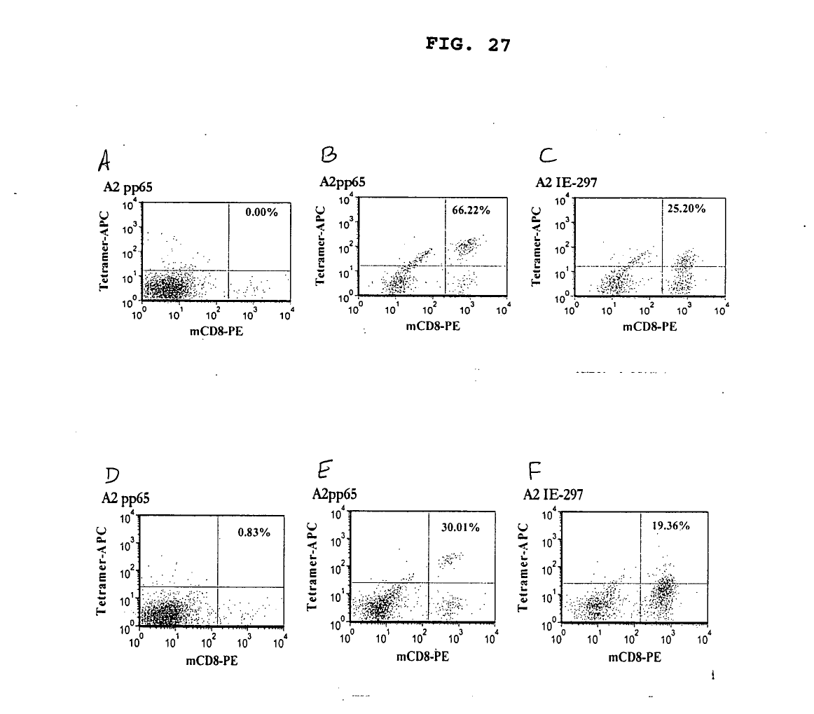

This assay was performed on splenocytes collected from immunized mice at the time of collection or after a 5-day stimulation with irradiated blast cells loaded with the indicated peptides (A2 pp 65 or A21E-297). Results are shown in FIG. 27, which shows flow cytometry analysis of splenocytes from immunized mice after tetramer binding. Panels show staining results for cells vaccinated in vivo with IEpp65mII DNA (A, B and C) or IEpp65mII-NLS-KO DNA (D, E and F). Panels A and D are cells which have not been subsequently stimulated in vivo. Panels B and E are cells which have been subjected to 5 days of in vitro stimulation with pp 65p 495 (NLVPMVATV; SEQ ID NO: 30); panels C and F are cells which have been subjected to 5 days of in vitro stimulation with IEmix, a mixture of IE immunogenic peptides. The results show that, using either vector (native or NLS-KO) CTL are raised that are specific to each CMV protein, pp 65 and IE1. The cells are memory cells since stimulation is necessary for their detection.

### Example 10

### Diagnostic Methods

To test CMV seropositivity, human fibroblast cells are infected with CMV and exposed to human sera. For example, human fibroblasts are either transfected or transduced with pp 65-NLS-KO. The resultant cells express the pp 65 protein (KO) and are used as a reagent in a diagnostic assay to display a large amount of CMV protein without risk of infectivity. The cells are used in hemagglutination assays, indirect fluorescent antibody assays, ELISAs and the like, according to methods of the art, to detect the presence of antibodies.

### REFERENCES

All references listed below are hereby incorporated by reference in their entirety.
1. Yao et al., “Site-directed mutation in a conserved kinase domain of human cytomegalovirus-pp 65 with preservation of cytotoxic T lymphocyte targeting.” Vaccine 19:1628-35, 2001.
2. Schmolke et al., “Nuclear targeting of the tegument protein pp 65 (UL83) of human cytomegalovirus: an unusual bipartite nuclear localization signal functions with other portions of the protein to mediate its efficient nuclear transport.” J. Virol. 69:1071-8, 1995.
3. Gallez-Hawkins et al., “DNA and low titer, helper-free, recombinant AAV prime-boost vaccination for cytomegalovirus induces an immune response to CMV-pp 65 and CMV-IE1 in transgenic HLA A*0201 mice.” Vaccine 23:819-26, 2004.
4. Pascolo S., “HLA class I transgenic mice: development, utilisation and improvement.” Expert Opin. Biol. Ther. 5:919-38, 2005.
5. Pascolo S. et al., “HLA-A2.1-restricted education and cytolytic activity of CD8 (+) T lymphocytes from beta2 microglobulin (beta2m) HLA-A2.1 monochain transgenic H-2 Db beta2m double knockout mice.” J. Exp. Med. 185:2043-51, 1997.
6. Gallez-Hawkins et al., “Kinase-deficient CMVpp65 triggers a CMVpp65 specific T-cell immune response in HLA-A*0201.Kb transgenic mice after DNA immunization.” Scand. J. Immunol. 55:592-8, 2002.
7. Benmohamed et al., “Induction of CTL response by a minimal epitope vaccine in HLA A*0201/DR1 transgenic mice: dependence on HLA class II restricted T(H) response.” Hum Immunol. 61:764-79, 2000.
8. Lacey et al., “Characterization of cytotoxic function of CMV-pp 65-specific CD8+ T-lymphocytes identified by HLA tetramers in recipients and donors of stem-cell transplants.” Transplantation 74:722-32, 2002.

1. An isolated nucleic acid that encodes an immunogenic peptide sequence, wherein the native sequence of said peptide contains a nuclear localization signal (NLS), and wherein the nucleic acids encoding said NLS have been deleted or mutated.

2. An isolated nucleic acid of claim 1, wherein said immunogenic peptide sequence is a cytomegalovirus protein sequence or derived from a cytomegalovirus protein sequence.

3. An isolated nucleic acid of claim 2, wherein said cytomegalovirus protein is selected from the group consisting of pp 65, pp 65mII, IE1 and a fusion of pp 65 and IE1.

4. An isolated nucleic acid of claim 3, wherein said cytomegalovirus protein is pp 65.

5. An isolated nucleic acid of claim 4, wherein nucleotides encoding amino acids 537-561 of pp 65 are deleted.

6. An isolated nucleic acid of claim 4, wherein adjacent regions to the nucleotides encoding amino acids 537-561 of pp 65 are deleted.

7. An isolated nucleic acid of claim 3, wherein said cytomegalovirus protein is IE1.

8. An isolated nucleic acid of claim 7, wherein nucleotides encoding amino acids 326-342 of IE1 are deleted.

9. An isolated nucleic acid of claim 8, wherein adjacent regions to the nucleotides encoding amino acids 326-342 of IE1 are deleted.

10. An isolated nucleic acid of claim 3, wherein said cytomegalovirus protein is a pp 65/IE1 fusion protein.

11. A polypeptide encoded by the nucleic acid of claim 1.

12. A polypeptide encoded by the nucleic acid of claim 5.

13. A polypeptide encoded by the nucleic acid of claim 8.

14. A polypeptide encoded by the nucleic acid of claim 10.

15. A method for vaccinating a subject in need thereof which comprises administering to said subject a nucleic acid of claim 1.

16. A method of vaccinating a subject in need thereof which comprises administering to said subject a polypeptide of claim 11.

17. A method of vaccinating a subject in need thereof which comprises administering to said subject a polypeptide encoded by the nucleic acid of claim 10.

18. An isolated nucleic acid of claim 1, wherein said immunogenic peptide comprises a viral peptide sequence.

19. A polypeptide encoded by the nucleic acid of claim 18.

20. A method for diagnosis of prior infection with a virus in a subject which comprises obtaining a sample of lymphocytes of said subject; contacting said sample in vitro with a polypeptide of claim 19 and determining whether said polypeptide stimulates said lymphocytes, wherein stimulation indicates prior infection with said virus.

21. A method for diagnosis of prior infection with a virus in a subject which comprises obtaining a sample of serum or plasma of said subject and contacting said sample in vitro with human fibroblasts that express pp 65-NLS-KO.

22. In a method of determining whether a serum or plasma sample contains antibody to a virus, the improvement which comprises using human fibroblasts that express pp 650NLS-KO as a reagent.

23. A method of producing virus-specific target cells for diagnosis of exposure to a virus or determination of virus cytotoxic lymphocyte function, which comprises contacting targets cells with a polypeptide of claim 19.

24. A method of expanding T cells specific for an antigen, which comprises contacting said T cells in vitro with a polypeptide of claim 11.

25. A method for making a recombinant vaccine that encodes a viral target polypeptide which localizes to the nucleus, the improvement which comprises knocking out the nuclear localization signals of said viral target polypeptide.

26. A method of improving protein expression of a recombinant protein that contains a NLS, which comprises knocking out said NLS.
